(19) United States
(12) Patent Application Publication
(10) Pub. No.: US 2020/0241006 A1
(43) Pub. Date: Jul. 30, 2020

(54) METHODS FOR MONITORING VEDOLIZUMAB TREATMENT

(71) Applicant: PROMETHEUS BIOSCIENCES, INC., San Diego, CA (US)

(72) Inventors: Snehal U. NAIK, San Diego, CA (US); Anjali JAIN, San Diego, CA (US)

(21) Appl. No.: 16/755,090

(22) PCT Filed: Oct. 9, 2018
(86) PCT No.: PCT/IB2018/057827

§ 371 Date: Apr. 9, 2020

Related U.S. Application Data
(60) Provisional application No. 62/570,530, filed on Oct. 10, 2017.
(60) Provisional application No. 62/593,056, filed on Nov. 30, 2017.

(51) Int. Cl. G01N 33/68

(52) U.S. Cl. CPC G01N 33/6893; G01N 2800/52; G01N 2800/065; G01N 2333/525

(57) ABSTRACT

The disclosure provides a method for predicting that a subject having inflammatory bowel disease (IBD) will have a clinical response or remission to an anti-α4β7 integrin during the course of therapy by assessing the concentration of the anti-α4β7 integrin drug at the induction or maintenance phase, respectively in a sample from the subject. The disclosure also provides a method for predicting whether a subject having inflammatory bowel disease (IBD) will be a remitter to an anti-α4β7 integrin drug treatment regimen by detecting the presence or level of at least one predictive marker.

### CROSS-REFERENCES TO RELATED APPLICATIONS

This application claims priority to U.S. Provisional Patent Application No. 62/570,530, filed Oct. 10, 2017, and U.S. Provisional Patent Application No. 62/593,056, filed Nov. 30, 2017, which applications are incorporated herein by reference in their entireties for all purposes.

### BACKGROUND OF THE INVENTION

Inflammatory bowel disease (IBD), which occurs worldwide and afflicts millions of people, is the collective term used to describe three gastrointestinal disorders of unknown etiology: Crohn's disease (CD), ulcerative colitis (UC), and indeterminate colitis (IC). IBD, together with irritable bowel syndrome (IBS), will affect one-half of all Americans during their lifetime, at a cost of greater than $2.6 billion dollars for IBD and greater than $8 billion dollars for IBS. A primary determinant of these high medical costs is the difficulty of diagnosing digestive diseases and how these diseases will progress. The cost of IBD and IBS is compounded by lost productivity, with people suffering from these disorders missing at least 8 more days of work annually than the national average.

Despite the successes of anti-TNFα therapies in the treatment of IBD, a subpopulation of patients are refractory to treatment, highlighting an unmet medical need for new therapies. Vedolizumab is a gut-specific, α4β7 integrin-neutralizing monoclonal Ab, which does not affect peripheral blood cell counts and appears to lack systemic effects. Vedolizumab is a new anti-inflammatory treatment option for the management of therapy-refractory patients.

There is a need in the art for methods of therapeutic management of diseases such as ulcerative colitis and Crohn's Disease using an individualized approach to monitor drug efficacy and optimize therapy accordingly.

### BRIEF SUMMARY OF THE INVENTION

The present disclosure provides methods for predicting that a subject having inflammatory bowel disease (IBD) will have a clinical response to an anti-α4β7 integrin drug or reach remission during the course of therapy. The method comprises administering to a subject having IBD an anti-α4β7 integrin drug during an induction phase; assessing the concentration of the anti-α4β7 integrin drug at the induction phase in a sample from the subject; and determining whether the subject will have a clinical response or reach remission at a later time point based upon the concentration of the anti-α4β7 integrin drug at the induction phase. In some embodiments, the inflammatory bowel disease is ulcerative colitis (UC) or Crohn's Disease (CD). In some embodiments, the anti-α4β7 integrin drug is ENTYVIO® (i.e., vedolizumab, VDZ).

In some embodiments, the induction phase is between week 0 and week 6 of the course of therapy. In some embodiments, the later time point is at weeks 8, 10, 12, 14, 16, 20, 22, 24, 30, 32, 40, 48, or 52 during the course of therapy. In some embodiments, a concentration of VDZ of ≥23.2 μg/ml at week 2 is associated with a clinical response or remission at week 14, 22, 30 and 52. In some embodiments, a concentration of VDZ of ≥19.8 μg/ml at week 6 is associated with a clinical response or remission at week 14, 22, 30 and 52. In some embodiments, the clinical response or remission is a member selected from the group of steroid free remission, clinical remission, normalized C-reactive protein (CRP), no steroid use in 4 weeks, and endoscopic remission. In some embodiments, a concentration of VDZ is negatively correlated to concentration of CRP at week 14 and 22. In some embodiments, the concentration of VDZ at induction is used to identify a subject that will have a clinical response or reach remission.

Also provided herein is a method for predicting that a subject having inflammatory bowel disease (IBD) being administered an anti-α4β7 integrin drug therapy will reach remission. The method comprises assessing the concentration of the anti-α4β7 integrin drug in a sample from the subject during a maintenance phase; and determining whether the subject will reach remission at a later time point based upon the concentration of the anti-α4β7 integrin drug during the maintenance phase. In some embodiments, the inflammatory bowel disease is ulcerative colitis (UC) or Crohn's Disease (CD). In some embodiments, the anti-α4β7 integrin drug is ENTYVIO® (vedolizumab, VDZ). In some embodiments, an induction phase is between week 0 and week 6 of the course of therapy, and the maintenance phase is after 6 weeks.

In some embodiments, the remission is a member selected from the group of steroid free remission, clinical remission, normalized C-reactive protein (CRP), no steroid use in 4 weeks, and endoscopic remission. In some embodiments, a concentration of VDZ of ≥12 μg/ml after week 6 is associated with remission at week 14, 22, 30, and 52, or later. In some embodiments, a concentration of VDZ of ≥13 μg/ml after week 6 is associated with remission at week 14, 22, 30, and 52, or later. In some embodiments, a concentration of VDZ of ≥14 μg/ml after week 6 is associated with remission at week 14, 22, 30, and 52, or later. In some embodiments, a concentration of VDZ of ≥15 μg/ml after week 6 is associated with remission at week 14, 22, 30, and 52, or later.

Also provided herein is a method for predicting that a subject having inflammatory bowel disease (IBD) will be a remitter to an anti-α4β7 integrin drug treatment regimen. The method comprises (a) detecting the presence or level of at least one predictive marker selected from the group of s-TNFα, s-α4β7, s-MAdCAM-1, s-CRP, s-AA, s-VCAM-1, s-ICAM-1, or a combination thereof, in a sample from the subject; and (b) classifying the subject as a remitter or a non-remitter to the anti-α4β7 integrin drug treatment according to a predictive marker profile based on a higher or lower level of the at least one predictive marker compared to a corresponding reference value. In some embodiments, the anti-α4β7 integrin drug is ENTYVIO® (i.e., vedolizumab, VDZ). In some embodiments, the concentration of s-α4β7 is increased in remitters. In some embodiments, one or more members selected from the group of s-TNF, s-MAdCAM-1, s-ICAM-1, and s-VCAM-1 is lower in remitters. In some embodiments, during induction time points, s-TNF concentrations are lower in remitters. In some embodiments, during maintenance time points, s-α4β7 is higher in remitters and s-VCAM-1 is lower in remitters.

These and other objects, aspects and embodiments will become more apparent when read with the detailed description and figures that follow.

### BRIEF DESCRIPTION OF THE DRAWINGS

FIG. 1 provides a graph of the changes in median vedolizumab levels (μg/mL) in patients that achieved remission at week 22 compared to patients that did not achieve remission at week 22. The graph shows changes in the median vedolizumab levels at week 2, week 6 and week 14.

FIG. 2 provides a graph of vedolizumab trough levels (μg/mL) in patients that received combination vedolizumab therapy compared to patients that received only vedolizumab therapy (monotherapy) at week 2, week 6 and week 14.

FIG. 3 shows the median serum VDZ concentrations are higher in clinical responders versus non-responders at the end of induction (week 14).

FIG. 4 shows a comparison between VDZ levels at weeks 2, 6, 14, 22, and 30 in patients that were in remission by week 52 of therapy and patients not in remission by week 52 of therapy. Endoscopic remission was defined as mucosal healing, clinical remission, and normal C-reactive protein.

FIG. 5 shows a comparison between VDZ levels at weeks 2, 6, 14, 22, and 30 in patients on monotherapy and patients on combination therapy with an immunomodulatory.

FIG. 6 shows a comparison in remission rates after 52 weeks between patients with median VDZ levels ≥23.2 mcg/ml at week 2 and patients with median VDZ levels <23.2 mcg/ml at week 2.

FIG. 7 shows a comparison in remission rates after 52 weeks between patients with median VDZ levels ≥19.8 mcg/ml at week 6 and patients with median VDZ levels <19.8 mcg/ml at week 6.

FIG. 8 shows rates of remission at week 52 by serum VDZ quartile level at week 2.

FIG. 9 shows rates of remission at week 52 by serum VDZ quartile level at week 6.

FIG. 10 shows the association of increasing VDZ concentration quartiles with higher rates of corticosteroid-free clinical and biochemical remission (primary outcome) during maintenance therapy.

FIG. 11 shows increasing VDZ concentration quartiles and rates of corticosteroid-free endoscopic remission during maintenance therapy.

FIG. 12 shows the association of increasing VDZ concentration quartiles with higher rates of corticosteroid-free deep remission during maintenance therapy.

FIG. 13 shows changes in biomarkers with VDZ therapy after 26 weeks in UC patients with baseline biomarkers prior to vedolizumab therapy. (A) soluble-tumor necrosis factor (s-TNF) concentrations measured at weeks 0 (baseline), 2, 6, 14, and 26. (B) concentrations of s-α4β7 and soluble-mucosal vascular addressin cell adhesion molecule (s-MAdCAM-1) measured at weeks 0, 2, 6, 14, and 26. (C) serum amyloid A (s-AA) concentrations measured at weeks 0, 2, 6, 14, and 26. (D) soluble-vascular cell adhesion molecule-1 (s-VCAM-1) concentrations measured at weeks 0, 2, 6, 14, and 26.

FIG. 14 shows biomarker trends over 26 weeks for UC patients in clinical remission during maintenance and UC patients not in clinical remission, using a linear mixed effects model. (A) s-TNF concentrations measured at weeks 0 (baseline), 2, 6, 14, and 26 for UC patients in clinical remission and not in clinical remission. (B) s-α4β7 concentrations measured at weeks 0 (baseline), 2, 6, 14, and 26 for UC patients in clinical remission and not in clinical remission. (C) s-MAdCAM-1 concentrations measured at weeks 0 (baseline), 2, 6, 14, and 26 for UC patients in clinical remission and not in clinical remission. (D) CRP concentrations measured at weeks 0 (baseline), 2, 6, 14, and 26 for UC patients in clinical remission and not in clinical remission. (E) s-AA concentrations measured at weeks 0 (baseline), 2, 6, 14, and 26 for UC patients in clinical remission and not in clinical remission. (F) soluble-intracellular adhesion molecule-1 (s-ICAM-1) concentrations measured at weeks 0 (baseline), 2, 6, 14, and 26 for UC patients in clinical remission and not in clinical remission. (G) s-VCAM-1 concentrations measured at weeks 0 (baseline), 2, 6, 14, and 26 for UC patients in clinical remission and not in clinical remission. (H) VDZ concentrations measured at weeks 2, 6, 14, and 26 for UC patients in clinical remission and not in clinical remission.

FIG. 15 shows biomarker trends over 26 weeks for UC patients in endoscopic remission during maintenance and UC patients not in endoscopic remission, using a linear mixed effects model. (A) s-TNF concentrations measured at weeks 0 (baseline), 2, 6, 14, and 26 for UC patients in endoscopic remission and not in endoscopic remission. (B) s-α4β7 concentrations measured at weeks 0 (baseline), 2, 6, 14, and 26 for UC patients in endoscopic remission and not in endoscopic remission. (C) s-MAdCAM-1 concentrations measured at weeks 0 (baseline), 2, 6, 14, and 26 for UC patients in endoscopic remission and not in endoscopic remission. (D) CRP concentrations measured at weeks 0 (baseline), 2, 6, 14, and 26 for UC patients in endoscopic remission and not in endoscopic remission. (E) s-AA concentrations measured at weeks 0 (baseline), 2, 6, 14, and 26 for UC patients in endoscopic remission and not in endoscopic remission. (F) s-ICAM-1 concentrations measured at weeks 0 (baseline), 2, 6, 14, and 26 for UC patients in endoscopic remission and not in endoscopic remission. (G) s-VCAM-1 concentrations measured at weeks 0 (baseline), 2, 6, 14, and 26 for UC patients in endoscopic remission and not in endoscopic remission. (H) VDZ concentrations measured at weeks 2, 6, 14, and 26 for UC patients in endoscopic remission and not in endoscopic remission.

### DETAILED DESCRIPTION OF THE INVENTION

### I. Definitions

The term “inflammatory bowel disease” or “IBD” includes gastrointestinal disorders such as, e.g., Crohn's disease (CD), ulcerative colitis (UC), and indeterminate colitis (IC). Inflammatory bowel diseases (e.g., CD, UC, and IC) are distinguished from all other disorders, syndromes, and abnormalities of the gastroenterological tract, including irritable bowel syndrome (IBS).

The term “sample” as used herein includes any biological specimen obtained from a patient. Samples include, without limitation, whole blood, plasma, serum, red blood cells, white blood cells (e.g., peripheral blood mononuclear cells (PBMC), polymorphonuclear (PMN) cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample such as a biopsy of a site of inflammation (e.g., needle biopsy), and cellular extracts thereof. In some embodiments, the sample is whole blood or a fractional component thereof such as plasma, serum, or a cell pellet. In other embodiments, the sample is obtained by isolating PBMCs and/or PMN cells using any technique known in the art. In yet other embodiments, the sample is a tissue biopsy, e.g., from a site of inflammation such as a portion of the gastrointestinal tract or synovial tissue.

The term “marker” or “biomarker” includes any biochemical marker, serological marker, genetic marker, or other clinical or echographic characteristic that can be used predicting whether a subject having inflammatory bowel disease (IBD) will respond to vedolizumab treatment. The marker can be used to classify a sample from the subject is a responder or a non-responder to vedolizumab therapy. In some embodiments, the markers are utilized in combination with a statistical analysis to provide a prognosis of IBD in an individual.

The term “classifying” includes “to associate” or “to categorize” a sample with a disease state. In certain instances, “classifying” is based on statistical evidence, empirical evidence, or both. In certain embodiments, the methods and systems of classifying use a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

The term “individual,” “subject,” or “patient” typically refers to humans, but also to other animals including, e.g., other primates, rodents, canines, felines, equines, ovines, porcines, and the like.

The term “prognosis” includes a prediction of the probable course and outcome of UC or CD or the likelihood of recovery from the disease.

The term “monitoring the progression or regression of UC or CD” includes the use of the methods of the present disclosure to determine the disease state (e.g., severity of UC) of an individual. In some aspects, the methods of the present disclosure can also be used to predict the progression of UC or CD, e.g., by determining a likelihood for UC to progress either rapidly or slowly in an individual based on the presence or level of at least one marker in a sample. In other aspects, the methods of the present disclosure can also be used to predict the regression of UC, e.g., by determining a likelihood for UC to regress either rapidly or slowly in an individual based on the presence or level of at least one marker in a sample.

The term “course of therapy” includes any therapeutic approach taken to relieve or prevent one or more symptoms associated with UC or CD. The term encompasses administering any compound, drug, procedure, or regimen useful for improving the health of an individual with UC and includes any of the therapeutic agents as well as surgery. One skilled in the art will appreciate that either the course of therapy or the dose of the current course of therapy can be changed, e.g., based upon the methods of the present disclosure.

As used herein, the phrase “at a later time point” includes phrases such as “by a later time point” and “within the later time point or “future date.” For example, a method for predicting whether a subject will have a clinical response to an anti-α4β7 integrin drug, or reach remission, or develop autoantibodies to an anti-α4β7 integrin drug (e.g., vedolizumab) at a later time point during a course of therapy includes a method for predicting whether a subject will have a clinical response to an anti-α4β7 integrin drug, or reach remission, or develop autoantibodies to an anti-α4β7 integrin drug (e.g., vedolizumab) by the later time point during the course of therapy, as well as a method for predicting whether a subject will have a clinical response to an anti-α4β7 integrin drug, or reach remission, or develop autoantibodies to an anti-α4β7 integrin drug (e.g., vedolizumab) within the later time point during the course of therapy.

In “quartile analysis”, there are three numbers (values) that divide a range of data into four equal parts. The first quartile (also called the ‘lower quartile’) is the number below which lies the bottom 25 percent of the data. The second quartile (the ‘median’) divides the range in the middle and has 50 percent of the data below it. The third quartile (also called the ‘upper quartile’) has 75 percent of the data below it and the top 25 percent of the data above it. As a non-limiting example, quartile analysis can be applied to the concentration level of a marker such as an antibody or other protein marker described herein, such that a marker level in the first quartile (<25%) is assigned a value of 1, a marker level in the second quartile (25-50%) is assigned a value of 2, a marker level in the third quartile (51%-<75%) is assigned a value of 3, and a marker level in the fourth quartile (75%-100%) is assigned a value of 4.

As used herein, the terms “response,” “patient response,” or “subject response” refer to the outcome of a patient or subject undergoing anti-α4β7 integrin therapy. The response of the patient can be assessed at any time point during anti-α4β7 integrin therapy, and can be assessed using any endpoint indicating a benefit to the patent, including, without limitation, inhibition, to some extent, of IBD progression, including slowing down and complete arrest; reduction in the number of IBD episodes and/or symptoms; reduction in lesional size; inhibition (i.e., reduction, slowing down or complete stopping) of disease severity; relief, to some extent, of one or more symptoms associated with the disorder; and/or increase in the length of disease-free presentation following treatment. The term responsiveness refers to a measurable response, including clinical response, clinical remission, endoscopic response, and endoscopic remission. A patient or subject in clinical remission and/or endoscopic remission undergoing anti-α4β7 integrin therapy or having undergone anti-α4β7 integrin therapy is referred to as a “remitter.” A patient or subject not in clinical remission or endoscopic remission after having undergone anti-α4β7 integrin therapy is referred to as a “non-remitter.”

As used herein, the term “clinical remission” with reference to UC subjects refers to a partial Mayo score of 2 or less points and/or a physician global assessment (PGA) score of 0 and neither treatment discontinuation nor colectomy. Crohn's disease “clinical remission” refers to a Harvey Bradshaw Index (HBI) score of less than 5, such as 4, 3, 2, 1, 0.

As used herein, the term “endoscopic remission” with reference to UC subjects refers to a Mayo endoscopic sub-score (ESS) of <2 (i.e., 0 or 1) on endoscopy. Crohn's disease “endoscopic remission” refers to a Simple Endoscopic Score (SES) for Crohn's Disease <3 or absence of ulcerations in CD patients.

### II. Detailed Description of the Embodiments

The present disclosure provides methods for predicting whether a subject having inflammatory bowel disease (IBD) will have a clinical response to an anti-α4β7 integrin drug or reach remission during the course of therapy (e.g., VDZ treatment), the method comprises:
(a) administering to a subject having IBD an anti-α4β7 integrin drug (e.g., VDZ) during an induction phase;
(b) assessing the levels of the anti-α4β7 integrin drug (e.g., VDZ concentration) at the induction phase in a sample from the IBD patient subject; and
(c) determining whether the IBD patient will have a clinical response or reach remission at a later time point based on the levels of the anti-α4β7 integrin drug (e.g., VDZ) at the induction phase.

The present disclosure also provides methods for predicting whether a subject having IBD while being administered an anti-α4β7 integrin drug therapy (e.g., VDZ treatment) will reach remission, the method comprises:
(a) assessing the concentration of the anti-α4β7 integrin drug in a sample from the IBD subject during a maintenance phase; and
(b) determining whether the subject will reach remission at a later time point based upon the concentration of the anti-α4β7 integrin drug during the maintenance phase.

Additionally, the present disclosure also provides methods for predicting whether a subject having IBD will be a remitter to an anti-α4β7 integrin drug treatment regimen, the method comprises:
(a) measuring the presence or level of at least one predictive marker in a sample from the subject; and
(b) classifying the subject as a remitter or a non-remitter to the anti-α4β7 integrin drug treatment according to a predictive marker profile based on a higher or lower level of the at least one predictive marker compared to a corresponding reference value.

In certain aspects, anti-α4β7 integrin drug, vedolizumab (ENTYVIO®), is indicated in adult patients with moderately to severely active ulcerative colitis (UC) or Crohn's Disease (CD) who have had an inadequate response with, lost response to, or were intolerant to a tumor necrosis factor (TNF) therapy, or had an inadequate response with, were intolerant to, or demonstrated dependence on corticosteroids for inducing and maintaining clinical response, inducing and maintaining clinical remission, improving endoscopic appearance of the mucosa, and achieving corticosteroid-free remission.

Vedolizumab can be administered as an intravenous infusion over about 30 minutes. The recommended dosage of in adults with UC or CD is 300 mg administered by intravenous infusion at, for example, zero, two and six weeks as induction therapy, and then every two to eight weeks thereafter (i.e., 8 weeks, 14 weeks, 22 weeks, 26 weeks, etc.) as maintenance therapy. Alternatively, the maintenance therapy may include administering vedolizumab every 4 weeks. Therapy may be discontinued in patients who show no evidence of therapeutic benefit by week 14.

In some embodiments, the VDZ concentration of an IBD patient having been administered an anti-α4β7 integrin drug (e.g., VDZ) during the induction phase is predictive of whether the patient will have a clinical response to the anti-α4β7 integrin drug (e.g., VDZ) or reach remission during a later time point of therapy. In some embodiments, the induction phase of VDZ therapy is 0 to 10 weeks. In some embodiments, the induction phase of VDZ therapy is 0, 1, 2, 3, 4, 5, 6, 7, 8, 9, or 10 weeks. In some embodiments, the induction phase of VDZ therapy is 1-9 weeks, 2-8 weeks, 3-7 weeks, or 4 to 6 weeks. In some embodiments, the induction phase of VDZ therapy is 0-8 weeks, 0-7 weeks, 0-6 weeks, 0-5 weeks, 0-4 weeks, 0-3 weeks, 0-2 weeks, or 0-1 week. In some embodiments, the induction phase of VDZ therapy is 0 to 6 weeks.

In some embodiments, the later time point of VDZ therapy during which the VDZ concentration of the IBD patient during the induction phase is predictive of a clinical response or remission is 8 to 72 weeks. In some embodiments, the later time point of VDZ therapy is 8, 10, 12, 14, 16, 18, 20, 22, 24, 26, 28, 30, 32, 36, 42, 46, 52, 60, 68 and/or 72 weeks. In some embodiments, the later time point of VDZ therapy is 8-60 weeks, 10-52 weeks, 14-46 weeks, and/or 20 to 30 weeks. In some embodiments, the later time point of VDZ therapy is 8, 10, 12, 14, 16, 20, 22, 24, 30, 32, 40, and/or 52 weeks. In some embodiments, the later time point of VDZ therapy is 8 weeks, 10 weeks, 14, weeks, 22 weeks, 30 weeks, and/or 52 weeks. In some embodiments, the later time point of VDZ therapy is 14, weeks, 22 weeks, 30 weeks, and 52 weeks.

In some embodiments, an IBD patient undergoing VDZ therapy with VDZ levels of between 10 and 100 μg/ml or more during the induction phase is associated with a clinical response or remission at a later time point of VDZ therapy. In some embodiments, a VDZ concentration of 10 μg/ml, 15 μg/ml, 20 μg/ml, 25 μg/ml, 30 μg/ml, 40 μg/ml, 50 μg/ml, 60 μg/ml, 70 μg/ml, 80 μg/ml, 100 μg/ml, or more during the induction phase is associated with a clinical response or remission at a later time point of VDZ therapy. In some embodiments, a VDZ concentration of ≥15 μg/ml, ≥18 μg/ml, ≥20 μg/ml, ≥22 μg/ml, ≥24 μg/ml, ≥26 μg/ml, ≥28 μg/ml, ≥30 μg/ml, ≥35 μg/ml, or ≥40 μg/ml during the induction phase is associated with a clinical response or remission at a later time point of VDZ therapy. In some embodiments, a concentration of VDZ of ≥23.2 μg/ml at between week 0 and week 4 is associated with a clinical response or remission at week 14, 22, 30 and 52. In some embodiments, a concentration of VDZ of ≥19.8 μg/ml at between week 4 and week 10 is associated with a clinical response or remission at week 14, 22, 30 and 52. In some embodiments, a concentration of VDZ of ≥23.2 μg/ml at week 2 is associated with a clinical response or remission at week 14, 22, 30 and 52. In some embodiments, a concentration of VDZ of ≥19.8 μg/ml at week 6 is associated with a clinical response or remission at week 14, 22, 30 and 52.

In some embodiments, the VDZ concentration of an IBD patient having been administered an anti-α4β7 integrin drug (e.g., VDZ) during a maintenance phase of the anti-α4β7 integrin drug (e.g., VDZ) therapy is predictive of whether the patient will reach remission at a later time point. The maintenance phase of the anti-α4β7 integrin drug (e.g., VDZ) therapy occurs after an induction phase of the anti-α4β7 integrin drug (e.g., VDZ) therapy. In some embodiments, the induction phase of VDZ therapy is 0 to 10 weeks. In some embodiments, the induction phase of VDZ therapy is 0, 1, 2, 3, 4, 5, 6, 7, 8, 9, or 10 weeks. In some embodiments, the induction phase of VDZ therapy is 1-9 weeks, 2-8 weeks, 3-7 weeks, or 4 to 6 weeks. In some embodiments, the induction phase of VDZ therapy is 0-8 weeks, 0-7 weeks, 0-6 weeks, 0-5 weeks, 0-4 weeks, 0-3 weeks, 0-2 weeks, or 0-1 week. In some embodiments, the induction phase of VDZ therapy is 0 to 6 weeks.

In some embodiments, the maintenance phase of VDZ therapy, during which the VDZ concentration of the IBD patient is predictive of remission, begins after 1 to 30 weeks of VDZ therapy. In some embodiments, the maintenance phase of VDZ therapy begins after 1, 2, 3, 4, 5, 6, 7, 8, 9, 10, 12, 14, 16, 18, 20, 22, 24, 26, 28, or 30 weeks of VDZ therapy. In some embodiments, the maintenance phase of VDZ therapy begins after 1-26 weeks, 2-24 weeks, 3-22 weeks, 4-20 weeks, 5-18 weeks, 6-16 weeks, 7-14 weeks, 8-12 weeks, or 9-10 weeks of VDZ therapy. In some embodiments, the maintenance phase of VDZ therapy begins after 2 weeks, 4 weeks, 6 weeks, 8 weeks, 10 weeks, or 12 weeks of VDZ therapy. In some embodiments, the maintenance phase of VDZ therapy, during which the VDZ concentration of the IBD patient is predictive of remission, begins after 8 weeks of VDZ therapy. Alternatively, the maintenance phase of VDZ therapy, during which the VDZ concentration of the IBD patient is predictive of remission, begins after 6 weeks of VDZ therapy. In some embodiments, the induction phase is between week 0 and week 6 of the course of therapy and maintenance is after 6 weeks, such as every two to eight weeks thereafter (i.e., 8 weeks, 14 weeks, 22 weeks, 26 weeks, etc.). In some embodiments, the maintenance phase of therapy may include administering vedolizumab every 4 weeks.

In some embodiments, the later time point at which an IBD patient can reach remission, as predicted by the maintenance phase VDZ concentration, is 8 to 72 weeks, or later. In some embodiments, the later time point is 8, 10, 12, 14, 16, 18, 20, 22, 24, 26, 28, 30, 32, 36, 42, 46, 52, 60, 68, and 72 weeks, or later. In some embodiments, the later time point is 8-70 weeks or later, 9-68 weeks or later, 10-64 weeks or later, 12-56 weeks or later, 14-52 weeks or later, 18-46 weeks or later, 20-42 weeks or later, and 22-30 weeks or later. In some embodiments, the later time point at which an IBD patient can reach remission, as predicted by the maintenance phase VDZ concentration, is 8, 10, 12, 14, 16, 20, 22, 24, 30, 32, 40, and 52 weeks, or later. In some embodiments, the later time point at which an IBD patient can reach remission, as predicted by the maintenance phase VDZ concentration, is 8 weeks, 10 weeks, 14, weeks, 22 weeks, 30 weeks, and 52 weeks, or later. In some embodiments, the later time point at which an IBD patient can reach remission, as predicted by the maintenance phase VDZ concentration, is 14, weeks, 22 weeks, 30 weeks, and 52 weeks, or later.

In some embodiments, an IBD patient undergoing anti-α4β7 integrin drug (e.g., VDZ) therapy with VDZ levels of between 5 and 100 μg/ml or more during the maintenance phase is associated with remission at a later time point. In some embodiments, a VDZ concentration of 5 μg/ml, 6 μg/ml, 8 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 22 μg/ml, 25 μg/ml, 30 μg/ml, 35 μg/ml, 40 μg/ml, 50 μg/ml, 60 μg/ml, 70 μg/ml, 80 μg/ml, 90 μg/ml, or more during the maintenance phase is associated with remission at a later time point. In some embodiments, a VDZ concentration of ≥10 μg/ml, ≥11 μg/ml, ≥12 μg/ml, ≥13 μg/ml, ≥14 μg/ml, ≥15 μg/ml, ≥16 μg/ml, ≥17 μg/ml, ≥18 μg/ml, ≥20 μg/ml, ≥22 μg/ml, ≥24 μg/ml, ≥28 μg/ml, ≥32 μg/ml, ≥35, or ≥40 μg/ml during the maintenance phase is associated with remission at a later time point.

In some embodiments, a concentration of VDZ of ≥12 μg/ml after 4 or more weeks is associated with remission at a later time point. In some embodiments, a concentration of VDZ of ≥13 μg/ml after 4 or more weeks is associated with remission at a later time point. In some embodiments, a concentration of VDZ of ≥14 μg/ml after 4 or more weeks is associated with remission at a later time point. In some embodiments, a concentration of VDZ of ≥15 μg/ml after 4 or more weeks is associated with remission at a later time point. In some embodiments, a concentration of VDZ of ≥12 μg/ml after week 6 is associated with remission at week 14, 22, 30, and 52, or later. In some embodiments, a concentration of VDZ of ≥13 μg/ml after week 6 is associated with remission at week 14, 22, 30, and 52, or later. In some embodiments, a concentration of VDZ of ≥14 μg/ml after week 6 is associated with remission at week 14, 22, 30, and 52, or later. In some embodiments, a concentration of VDZ of ≥15 μg/ml after week 6 is associated with remission at week 14, 22, 30, and 52, or later.

A. Inflammatory Bowel Disease (IBD)

Inflammatory bowel disease (IBD) is a group of inflammatory conditions of the large intestine and small intestine. The main forms of IBD are Crohn's disease (CD) and ulcerative colitis (UC). Other less common forms of IBD include, e.g., indeterminate colitis (IC), collagenous colitis, lymphocytic colitis, ischemic colitis, diversion colitis, Behcet's syndrome, infective colitis, and the like. U.S. Patent Publication 2008/0131439, entitled “Methods of Diagnosing Inflammatory Bowel Disease” is incorporated herein by reference for all purposes.

1. Crohn's Disease

Crohn's disease (CD) is a disease of chronic inflammation that can involve any part of the gastrointestinal tract. Commonly, the distal portion of the small intestine, i.e., the ileum, and the cecum are affected. In other cases, the disease is confined to the small intestine, colon, or anorectal region. CD occasionally involves the duodenum and stomach, and more rarely the esophagus and oral cavity.

The variable clinical manifestations of CD are, in part, a result of the varying anatomic localization of the disease. The most frequent symptoms of CD are abdominal pain, diarrhea, and recurrent fever. CD is commonly associated with intestinal obstruction or fistula, an abnormal passage between diseased loops of bowel. CD also includes complications such as inflammation of the eye, joints, and skin, liver disease, kidney stones, and amyloidosis. In addition, CD is associated with an increased risk of intestinal cancer.

Several features are characteristic of the pathology of CD. The inflammation associated with CD, known as transmural inflammation, involves all layers of the bowel wall. Thickening and edema, for example, typically also appear throughout the bowel wall, with fibrosis present in long-standing forms of the disease. The inflammation characteristic of CD is discontinuous in that segments of inflamed tissue, known as “skip lesions,” are separated by apparently normal intestine. Furthermore, linear ulcerations, edema, and inflammation of the intervening tissue lead to a “cobblestone” appearance of the intestinal mucosa, which is distinctive of CD.

A hallmark of CD is the presence of discrete aggregations of inflammatory cells, known as granulomas, which are generally found in the submucosa. Some CD cases display typical discrete granulomas, while others show a diffuse granulomatous reaction or a nonspecific transmural inflammation. As a result, the presence of discrete granulomas is indicative of CD, although the absence of granulomas is also consistent with the disease. Thus, transmural or discontinuous inflammation, rather than the presence of granulomas, is a preferred diagnostic indicator of CD (Rubin and Farber, Pathology (Second Edition), Philadelphia, J.B. Lippincott Company (1994)).

Crohn's disease may be categorized by the behavior of disease as it progresses. This was formalized in the Vienna classification of Crohn's disease. See, Gasche et al., Inflamm. Bowel Dis., 6:8-15 (2000). There are three categories of disease presentation in Crohn's disease: (1) stricturing, (2) penetrating, and (3) inflammatory. Stricturing disease causes narrowing of the bowel which may lead to bowel obstruction or changes in the caliber of the feces. Penetrating disease creates abnormal passageways (fistulae) between the bowel and other structures such as the skin. Inflammatory disease (also known as non-stricturing, non-penetrating disease) causes inflammation without causing strictures or fistulae.

As such, Crohn's disease represents a number of heterogeneous disease subtypes that affect the gastrointestinal tract and may produce similar symptoms. As used herein in reference to CD, the term “clinical subtype” includes a classification of CD defined by a set of clinical criteria that distinguish one classification of CD from another. As non-limiting examples, subjects with CD can be classified as having stricturing (e.g., internal stricturing), penetrating (e.g., internal penetrating), or inflammatory disease as described herein, or these subjects can additionally or alternatively be classified as having fibrostenotic disease, small bowel disease, internal perforating disease, perianal fistulizing disease, UC-like disease, the need for small bowel surgery, the absence of features of UC, or combinations thereof.

In certain instances, subjects with CD can be classified as having complicated CD, which is a clinical subtype characterized by stricturing or penetrating phenotypes. In certain other instances, subjects with CD can be classified as having a form of CD characterized by one or more of the following complications: fibrostenosis, internal perforating disease, and the need for small bowel surgery. In further instances, subjects with CD can be classified as having an aggressive form of fibrostenotic disease requiring small bowel surgery. Criteria relating to these subtypes have been described, for example, in Gasche et al., Inflamm. Bowel Dis., 6:8-15 (2000); Abreu et al., Gastroenterology, 123:679-688 (2002); Vasiliauskas et al., Gut, 47:487-496 (2000); Vasiliauskas et al., Gastroenterology, 110:1810-1819 (1996); and Greenstein et al., Gut, 29:588-592 (1988).

The “fibrostenotic subtype” of CD is a classification of CD characterized by one or more accepted characteristics of fibrostenosing disease. Such characteristics of fibrostenosing disease include, but are not limited to, documented persistent intestinal obstruction or an intestinal resection for an intestinal obstruction. The fibrostenotic subtype of CD can be accompanied by other symptoms such as perforations, abscesses, or fistulae, and can further be characterized by persistent symptoms of intestinal blockage such as nausea, vomiting, abdominal distention, and inability to eat solid food. Intestinal X-rays of patients with the fibrostenotic subtype of CD can show, for example, distention of the bowel before the point of blockage.

The requirement for small bowel surgery in a subject with the fibrostenotic subtype of CD can indicate a more aggressive form of this subtype. Additional subtypes of CD are also known in the art and can be identified using defined clinical criteria. For example, internal perforating disease is a clinical subtype of CD defined by current or previous evidence of entero-enteric or entero-vesicular fistulae, intra-abdominal abscesses, or small bowel perforation. Perianal perforating disease is a clinical subtype of CD defined by current or previous evidence of either perianal fistulae or abscesses or rectovaginal fistula. The UC-like clinical subtype of CD can be defined by current or previous evidence of left-sided colonic involvement, symptoms of bleeding or urgency, and crypt abscesses on colonic biopsies. Disease location can be classified based on one or more endoscopic, radiologic, or pathologic studies.

One skilled in the art understands that overlap can exist between clinical subtypes of CD and that a subject having CD can have more than one clinical subtype of CD. For example, a subject having CD can have the fibrostenotic subtype of CD and can also meet clinical criteria for a clinical subtype characterized by the need for small bowel surgery or the internal perforating disease subtype. Similarly, the markers described herein can be associated with more than one clinical subtype of CD.

2. Ulcerative Colitis

Ulcerative colitis (UC) is a disease of the large intestine characterized by chronic diarrhea with cramping, abdominal pain, rectal bleeding, loose discharges of blood, pus, and mucus. The manifestations of UC vary widely. A pattern of exacerbations and remissions typifies the clinical course for about 70% of UC patients, although continuous symptoms without remission are present in some patients with UC. Local and systemic complications of UC include arthritis, eye inflammation such as uveitis, skin ulcers, and liver disease. In addition, UC, and especially the long-standing, extensive form of the disease is associated with an increased risk of colon carcinoma.

UC is a diffuse disease that usually extends from the most distal part of the rectum for a variable distance proximally. The term “left-sided colitis” describes an inflammation that involves the distal portion of the colon, extending as far as the splenic flexure. Sparing of the rectum or involvement of the right side (proximal portion) of the colon alone is unusual in UC. The inflammatory process of UC is limited to the colon and does not involve, for example, the small intestine, stomach, or esophagus. In addition, UC is distinguished by a superficial inflammation of the mucosa that generally spares the deeper layers of the bowel wall. Crypt abscesses, in which degenerated intestinal crypts are filled with neutrophils, are also typical of UC (Rubin and Farber, supra).

In certain instances, with respect to UC, the variability of symptoms reflect differences in the extent of disease (i.e., the amount of the colon and rectum that are inflamed) and the intensity of inflammation. Disease starts at the rectum and moves “up” the colon to involve more of the organ. UC can be categorized by the amount of colon involved. Typically, patients with inflammation confined to the rectum and a short segment of the colon adjacent to the rectum have milder symptoms and a better prognosis than patients with more widespread inflammation of the colon.

In comparison with CD, which is a patchy disease with frequent sparing of the rectum, UC is characterized by a continuous inflammation of the colon that usually is more severe distally than proximally. The inflammation in UC is superficial in that it is usually limited to the mucosal layer and is characterized by an acute inflammatory infiltrate with neutrophils and crypt abscesses. In contrast, CD affects the entire thickness of the bowel wall with granulomas often, although not always, present. Disease that terminates at the ileocecal valve, or in the colon distal to it, is indicative of UC, while involvement of the terminal ileum, a cobblestone-like appearance, discrete ulcers, or fistulas suggests CD.

The different types of ulcerative colitis are classified according to the location and the extent of inflammation. As used herein in reference to UC, the term “clinical subtype” includes a classification of UC defined by a set of clinical criteria that distinguish one classification of UC from another. As non-limiting examples, subjects with UC can be classified as having ulcerative proctitis, proctosigmoiditis, left-sided colitis, pancolitis, fulminant colitis, and combinations thereof. Criteria relating to these subtypes have been described, for example, in Kornbluth et al., Am. J. Gastroenterol., 99: 1371-85 (2004).

Ulcerative proctitis is a clinical subtype of UC defined by inflammation that is limited to the rectum. Proctosigmoiditis is a clinical subtype of UC which affects the rectum and the sigmoid colon. Left-sided colitis is a clinical subtype of UC which affects the entire left side of the colon, from the rectum to the place where the colon bends near the spleen and begins to run across the upper abdomen (the splenic flexure). Pancolitis is a clinical subtype of UC which affects the entire colon. Fulminant colitis is a rare, but severe form of pancolitis. Patients with fulminant colitis are extremely ill with dehydration, severe abdominal pain, protracted diarrhea with bleeding, and even shock.

In some embodiments, classification of the clinical subtype of UC is important in planning an effective course of treatment. While ulcerative proctitis, proctosigmoiditis, and left-sided colitis can be treated with local agents introduced through the anus, including steroid-based or other enemas and foams, pancolitis must be treated with oral medication so that active ingredients can reach all of the affected portions of the colon.

One skilled in the art understands that overlap can exist between clinical subtypes of UC and that a subject having UC can have more than one clinical subtype of UC. Similarly, the prognostic markers described herein can be associated with more than one clinical subtype of UC.

3. Patients with CD or UC

In some embodiments, the subjects of methods disclosed herein are patients with moderate to severe CD or a score of about 220 to 450 on the Crohn's Disease Activity Index (CDAI ranges from 0 to about 600, with higher scores indicating greater disease activity. In other embodiments, the subjects have moderate to severe UC or a Mayo Clinic score ranging from about 6 to 12 (Mayo Clinic scores range from 0 to 12 with higher scores indicating active disease), with a sigmoidoscopy sub-score of at least 2, and disease that extends 15 cm or more from the anal verge.

In some embodiments, the subject has not received an anti-α4β7 integrin drug (e.g., vedolizumab). In some embodiments, the subject has not received an anti-TNFα therapy. The subject may be predicted to be nonresponsive to an anti-TNFα drug. In other embodiments, the subject has developed an intolerance to the anti-TNFα drug. In some instances, the subject has had an inadequate response to the anti-TNFα drug. In other instances, the subject has lost response to the anti-TNFα drug.

In some aspects of the present disclosure, the method is performed at baseline (e.g., prior to receiving an anti-α4β7 integrin drug). The presence or level of one or more predictive markers described herein may be detected or quantitated at a single time point. In other aspects, the method is performed during induction therapy (e.g., at week 0 to week 6 of anti-α4β7 integrin drug treatment). In some embodiments, the presence or level of one or more predictive markers are measured at one or more time points during induction therapy. In yet other aspects, the method is performed during maintenance therapy (e.g., at week 8 or later of anti-α4β7 integrin drug treatment). In some instances, the presence or level of one or more predictive markers are measured at one or more time points during maintenance therapy.

B. Markers for Measuring Anti-α4β7 Integrin Drug and Anti-Drug Antibody (ADA) Levels

In some embodiments, the method comprises determining the presence and/or level of anti-α4β7 integrin drug (e.g., level of free anti-α4β7 integrin therapeutic antibody such as vedolizumab) and/or anti-drug antibody (ADA) (e.g., level of autoantibody to the anti-α4β7 integrin drug such as HAHA) in a patient sample (e.g., a serum sample from a patient on anti-α4β7 integrin drug therapy) at multiple time points, e.g., before, during, and/or after the course of therapy.

In some embodiments, the presence and/or level of anti-α4β7 integrin drug and/or ADA is determined with a homogeneous mobility shift assay (HMSA) using size exclusion chromatography. These methods are described in U.S. Pat. Nos. 8,574,855, and 8,865,417 and U.S. Patent Publication Nos. 2014/0051184 and 2014/0141983, the disclosures of which are hereby incorporated by reference in their entirety for all purposes. The methods are particularly useful for measuring the presence or level of α4β7 integrin inhibitors as well as autoantibodies (e.g., HACA, HAHA, etc.) that are generated against them.

In other embodiments, the presence and/or level of anti-α4β7 integrin drug and/or ATV is determined with an immunoassay, such as an enzyme-linked immunosorbent assay (ELISA). In yet other embodiments, the presence and/or level of anti-α4β7 integrin drug and/or ATV is determined with a flow cytometry assay such as FACS.

C. Markers for Predicting Remittance to an Anti-α4β7 Integrin Drug Treatment Regimen

A variety of IBD markers, including biochemical markers, serological markers, protein markers, genetic markers, and other clinical or echographic characteristics, are suitable for use in the methods of the present disclosure for predicting whether a subject having IBD will be a remitter to an anti-α4β7 integrin drug treatment regimen (e.g., VDZ therapy). In certain aspects, prognostic methods described herein utilize the application of an algorithm (e.g., statistical analysis) to the presence or concentration level determined for one or more of the markers to aid or assist in a prognosis regarding whether an IBD patient will be a remitter or a non-remitter to an anti-α4β7 integrin drug treatment regimen (e.g., VDZ therapy). In some embodiments, remittance or non-remittance to an anti-α4β7 integrin drug treatment regimen (e.g., VDZ therapy) is predictive in IBD patients with ulcerative colitis (UC).

The following markers are suitable for predicting whether a subject having inflammatory bowel disease (IBD) will be a remitter to an anti-α4β7 integrin drug treatment regimen. The markers can make up a marker profile. Suitable markers include, but are not limited to, tumor necrosis factor (TNF)-α, α4β7, mucosal addressin cell adhesion molecule (MAdCAM-1), C-reactive protein (CRP), amyloid A (AA), vascular cell adhesion molecule-1 (VCAM-1), intracellular adhesion molecule-1 (ICAM-1), and combinations thereof. In some embodiments, the markers suitable for predicting whether a subject having UC will be a remitter to an anti-α4137 integrin drug treatment regimen (e.g., VDZ therapy) are in their free soluble form, referred to as serum biomarkers. For example, serum or soluble (s) biomarkers for predicting remittance or non-remittance to an anti-α4β7 integrin drug treatment regimen (e.g., VDZ therapy) in a UC patient include, but are not limited to, s-TNF-α, s-α4β7, s-MAdCAM-1, s-CRP, s-AA, s-VCAM-1, s-ICAM-1, and combinations thereof.

In some embodiments, the methods provided herein include measuring and/or detecting the presence or level of one or more (e.g., 1, 2, 3, 4, 5, 6, 7, 8, 9, 10, 11, 12, 13, 14, 15, 16, 17, 18, 19, 20 or more) predictive markers. In some embodiments, the method for predicting whether a subject having UC will be a remitter to an anti-α4β7 integrin drug treatment regimen (e.g., VDZ therapy) includes detecting the presence or level of s-TNFα, s-α4β7, s-MAdCAM-1, s-CRP, s-AA, s-VCAM-1, s-ICAM-1, or combinations thereof. In some embodiments, the method includes detecting the presence or level of s-TNFα, s-α4β7, s-MAdCAM-1, s-AA, and s-VCAM-1. In some embodiments, the method includes detecting the presence or level of s-TNFα, s-α4β7, s-MAdCAM-1, s-AA, and s-VCAM-1. In some embodiments, the method includes detecting the presence or level of s-α4β7. In some embodiments, the method includes detecting the presence or level of s-α4β7. In some embodiments, the method includes detecting the presence or level of s-MAdCAM-1. In some embodiments, the method includes detecting the presence or level of s-TNFα. In some embodiments, the method includes detecting the presence or level of s-CRP. In some embodiments, the method includes detecting the presence or level of s-AA. In other embodiments, the method includes detecting the presence or level of s-ICAM-1. In other embodiments, the method includes detecting the presence or level of s-VCAM-1. In some embodiments, the method includes detecting the presence or level of s-TNFα, s-MAdCAM-1, s-ICAM-1, and s-VCAM-1.

In some embodiments, the method for predicting whether a subject having UC will be a remitter to an anti-α4β7 integrin drug treatment regimen (e.g., VDZ therapy) includes measuring and/or detecting the presence or level of one or more (e.g., 1, 2, 3, 4, 5, 6, 7, 8, 9, 10, 11, 12, 13, 14, 15, 16, 17, 18, 19, 20 or more) at one or more (e.g., 1, 2, 3, 4, 5, 6, 7, 8, 9, 10 or more) time points. In some embodiments, the method for predicting whether a subject having UC will be a remitter to an anti-α4β7 integrin drug treatment regimen (e.g., VDZ therapy) includes measuring and/or detecting the presence or level of one or more (e.g., 1, 2, 3, 4, 5, 6, 7, 8, 9, 10, 11, 12, 13, 14, 15, 16, 17, 18, 19, 20 or more) at, for example, baseline (prior to the administration of VDZ), post week 0 (immediately after the initial administration of the drug), week 2, week 4, week 6, week 8, week 10, week 12, week 14, week 16, week 18, week 20, week 22, week 24, week 26, week 28, week 30, week 32, week 34, week 36, week 38, week 40, week 42, week 44, week 46, week 48, week 50, or week 52 of drug treatment, or any combination thereof. In some embodiments, the presence or level of one or more predictive markers (e.g., 1, 2, 3, 4, 5, 6, 7, 8, 9, 10, 11, 12, 13, 14, 15, 16, 17, 18, 19, 20 or more) is measured once a week or less often during the course of the anti-α4β7 integrin drug treatment regimen.

In some embodiments, the method for predicting whether a subject having UC will be a remitter to an anti-α4β7 integrin drug treatment regimen (e.g., VDZ therapy) includes measuring and/or detecting the presence or level of one or more (e.g., 1, 2, 3, 4, 5, 6, 7, 8, 9, 10, 11, 12, 13, 14, 15, 16, 17, 18, 19, 20 or more) predictive markers at baseline. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected at week 2. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected at week 4. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected at week 6. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected at week 10. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected at week 14. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected at week 26. In some embodiments, the presence or level of one or more (e.g., 1, 2, 3, 4, 5, 6, 7, 8, 9, 10, 11, 12, 13, 14, 15, 16, 17, 18, 19, 20 or more) predictive markers are detected after week 26.

In certain instances, the presence or level of a particular biomarker is detected at the level of mRNA expression with an assay such as, for example, a hybridization assay or an amplification-based assay. In certain other instances, the presence or level of a particular biomarker is detected at the level of protein expression using, for example, an immunoassay (e.g., ELISA), an immunohistochemical assay or a proximity dual detection assay. Suitable ELISA kits for determining the presence or level of a biomarker in a sample such as a serum, plasma, saliva, or urine sample are available from, e.g., R&D Systems, Inc. (Minneapolis, Minn.), Neogen Corp. (Lexington, Ky.), Alpco Diagnostics (Salem, N.H.), Assay Designs, Inc. (Ann Arbor, Mich.), BD Biosciences Pharmingen (San Diego, Calif.), Invitrogen (Camarillo, Calif.), Calbiochem (San Diego, Calif.), CHEMICON International, Inc. (Temecula, Calif.), Antigenix America Inc. (Huntington Station, N.Y.), QIAGEN Inc. (Valencia, Calif.), Bio-Rad Laboratories, Inc. (Hercules, Calif.), and/or Bender MedSystems Inc. (Burlingame, Calif.). In some embodiments, the proximity dual detection assay is a CEER™ (Collaborative Enzyme Enhanced Reactive innumoassay) assay, an antibody-microarray based platform is utilized to form a unique “triple-antibody-enzyme-channeling” immuno-complex capable of measuring analytes of limited availability in a sample. A detailed description of CEER™ is found in, e.g., U.S. Pat. No. 8,163,499, which is hereby incorporated by reference in its entity for all purposes.

1. TNFα

The term “TNFα” or “tumor necrosis factor α” refers to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 225, 230 or more amino acids, to a human TNFα sequence shown below; (2) bind to antibodies, e.g., polyclonal antibodies, raised against an immunogen comprising an amino acid sequence shown below, or conservatively modified variants thereof; (3) bind to a TNFα binding protein; (4) compete with a naturally occurring TNFα ligand binding to a TNFα ligand binding protein; (5) induce apoptosis in cells having a membrane-bound TNFα binding protein; (6) specifically hybridize under stringent hybridization conditions to a nucleic acid sequence shown below, or conservatively modified variants thereof; (7) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human TNFα mRNA sequence; and/or (8) have at least 25, often 50, 75, 100, 125 or 143 contiguous amino acid residues of a human TNFα polypeptide sequence. The human TNFα polypeptide sequence is set forth in, e.g., Genbank Accession No. NP_000585. The human TNFα mRNA (coding) sequence is set forth in, e.g., Genbank Accession No. NM_000594. One skilled in the art will appreciate that variants, isoforms, alternative sequences of TNFα are also useful in the present disclosure.

The terms “s-TNFα” or “serum TNFα” refer to the free soluble form of tumor necrosis factor α. In some embodiments, an immunoassay such as a sandwich assay or ELISA can be used to measure TNFα, or more specifically, s-TNFα. Non-limiting examples include Human TNFα High Sensitivity ELISA (Cat. No. BMS223HS, eBioscience, San Diego, Calif.), Erenna Human TNFα immunoassay (Cat. No. 03-0022-xx, Singulex, Alameda, Calif.), Human TNFα cytokine assay (Cat. No. K151BHA-5, Meso Scale Diagnostics (MSD), Rockville, Md.)) and a muli-marker immunoassay (e.g., as described in U.S. Pat. No. 8,450,069; Singulex).

2. α4β7

The term “α4β7 integrin” refers to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 250, or 281 amino acids, to a human α4β7 integrin sequence shown below; (2) bind to antibodies, e.g., polyclonal antibodies, raised against an immunogen comprising an amino acid sequence shown below, or conservatively modified variants thereof; (3) bind to an α4β7 integrin binding protein; (4) specifically hybridize under stringent hybridization conditions to a nucleic acid sequence shown below, or conservatively modified variants thereof; (5) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human α4β7 integrin mRNA sequence; and/or (6) have at least 25, often 50, 75, 100, 125 or 143 contiguous amino acid residues of a human α4β7 integrin polypeptide sequence. The human α4β7 integrin polypeptide sequence is set forth in, e.g., Genbank Accession Nos. NP_000876 and NP_000880. The human α4β7 integrin mRNA (coding) sequence is set forth in, e.g., Genbank Accession Nos. NM_000885 and NM_000889. One skilled in the art will appreciate that variants, isoforms, alternative sequences of α4β7 integrin are also useful in the present disclosure. The terms “s-α4β7” or “serum α4β7” refer to the free soluble form of human α4β7 integrin.

3. MAdCAM-1

The term “mucosal addressin cell adhesion molecule” or “MAdCAM-1” refers to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 250, 281, 290 or more amino acids, to a human MAdCAM-1 sequence shown below; (2) bind to antibodies, e.g., polyclonal antibodies, raised against an immunogen comprising an amino acid sequence shown below, or conservatively modified variants thereof; (3) bind to a MAdCAM-1 binding protein; (4) specifically hybridize under stringent hybridization conditions to a nucleic acid sequence shown below, or conservatively modified variants thereof; (5) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human MAdCAM-1 mRNA sequence; and/or (6) have at least 25, often 50, 75, 100, 125 or 143 contiguous amino acid residues of a human MAdCAM-1 polypeptide sequence.

MAdCAM-1 is a predictive marker which is essential in mediating the infiltration of leucocytes into chronically inflamed tissues and plays a pivotal role in T-lymphocyte homing to the gut. The human MAdCAM-1 polypeptide sequence is set forth in, e.g., Genbank Accession No. NP_570118. The human MAdCAM-1 mRNA (coding) sequence is set forth in, e.g., Genbank Accession No. NM_130762. One skilled in the art will appreciate that variants, isoforms, alternative sequences of MAdCAM-1 are also useful in the present disclosure.

The terms “s-MAdCAM-1” or “serum MAdCAM-1” refer to the free soluble form of the mucosal addressin cell adhesion molecule. MAdCAM-1 is expressed by intestinal endothelium and its expression is increased under conditions of inflammation, including in the setting of inflammatory bowel disease (IBD). This molecule has been detected in body fluids, such as urine and serum, using a sandwich ELISA assay; however, the mechanism by which it is cleaved from the endothelial surface and released into circulation as soluble s-MAdCAM-1 is not well defined.

4. Acute Phase Proteins

The determination of the presence or level of one or more acute-phase proteins in a sample is also useful in the present disclosure. Acute-phase proteins are a class of proteins whose plasma concentrations increase (positive acute-phase proteins) or decrease (negative acute-phase proteins) in response to inflammation. This response is called the acute-phase reaction (also called acute-phase response). Examples of positive acute-phase proteins include, but are not limited to, C-reactive protein (CRP).

The term “CRP” or “C-reactive protein” refers to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 210, 220 or more amino acids, to a human CRP sequence shown below; (2) bind to antibodies, e.g., polyclonal antibodies, raised against an immunogen comprising an amino acid sequence shown below, or conservatively modified variants thereof; (3) bind to a CRP binding protein; (4) specifically hybridize under stringent hybridization conditions to a nucleic acid sequence shown below, or conservatively modified variants thereof; (5) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human CRP mRNA sequence; and/or (6) have at least 25, often 50, 75, 100, 125 or 143 contiguous amino acid residues of a human CRP polypeptide sequence.

CRP is a protein found in the blood in response to inflammation (an acute-phase protein). CRP is typically produced by the liver and by fat cells (adipocytes). It is a member of the pentraxin family of proteins. The human CRP polypeptide sequence is set forth in, e.g., Genbank Accession No. NP_000558. The human CRP mRNA (coding) sequence is set forth in, e.g., Genbank Accession No. NM_000567. One skilled in the art will appreciate that CRP is also known as PTX1, MGC88244, and MGC149895.

The terms “s-CRP” or “serum CRP” refer to the free soluble form of the C-reactive protein. In some embodiments, the presence or level of s-CRP is detected at the level of mRNA expression with an assay such as, for example, a hybridization assay or an amplification-based assay. In some embodiments, the presence or level of s-CRP is detected at the level of protein expression using, for example, an immunoassay (e.g., ELISA) or an immunohistochemical assay. For example, a sandwich colorimetric ELISA assay available from Alpco Diagnostics (Salem, N.H.) can be used to determine the level of CRP in a serum, plasma, urine, or stool sample. Similarly, an ELISA kit available from Biomeda Corporation (Foster City, Calif.) can be used to detect CRP levels in a sample. Other methods for determining CRP levels in a sample are described in, e.g., U.S. Pat. Nos. 6,838,250; 6,406,862; and 7,439,019; and U.S. Patent Publication No. 2006/0019410. Additional methods for determining CRP levels include, e.g., immunoturbidimetry assays, rapid immunodiffusion assays, and visual agglutination assays. Suitable ELISA kits for determining the presence or level of SAA in a sample such as serum, plasma, saliva, urine, or stool are available from, e.g., Antigenix America Inc. (Huntington Station, N.Y.), Abazyme (Needham, Mass.), USCN Life (Missouri City, Tex.), and/or U.S. Biological (Swampscott, Mass.).

5. s-AA

The terms “s-AA,” “serum AA,” or “serum amyloid A protein” refer to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 250, 300, 400, 500, 600, 700, 720, 730, or more amino acids; (2) bind to antibodies, e.g., polyclonal antibodies, raised against an immunogen comprising an amino acid sequence shown below, or conservatively modified variants thereof; (3) bind to a s-AA binding protein; (4) specifically hybridize under stringent hybridization conditions to a nucleic acid sequence shown below, or conservatively modified variants thereof; (5) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human s-AA mRNA sequence; and/or (6) have at least 25, often 50, 75, 100, 125 or 143 contiguous amino acid residues of a human s-AA polypeptide sequence.

s-AA is an inflammatory marker and an acute-phase reactant that is mainly transported as an apolipoprotein in high-density lipoprotein. s-AA is predominantly synthesized in the liver by hepatocytes in response to proinflammatory cytokines. See, Uhlar, C. M., et al. Scand. J. Immunol. 1999, 49(4), 399-404. The human s-AA polypeptide sequence is set forth in, e.g., human serum amyloid A [Homo sapiens]122 aa protein Accession: AAA60297.1. The human s-AA mRNA (coding) sequence is set forth in Accession: M10906, 369 bp; mRNA Human serum amyloid A (SAA) mRNA. s-AA levels can be assessed with enzyme-linked immunosorbent assays.

6. VCAM-1

The term “VCAM-1” or “vascular cell adhesion molecule 1” refers to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 250, 300, 400, 500, 600, 700, 720, 730, or more amino acids, to a human VCAM-1 sequence shown below; (2) bind to antibodies, e.g., polyclonal antibodies, raised against an immunogen comprising an amino acid sequence shown below, or conservatively modified variants thereof; (3) bind to a VCAM-1 binding protein; (4) specifically hybridize under stringent hybridization conditions to a nucleic acid sequence shown below, or conservatively modified variants thereof; (5) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human VCAM-1 mRNA sequence; and/or (6) have at least 25, often 50, 75, 100, 125 or 143 contiguous amino acid residues of a human VCAM-1 polypeptide sequence.

VCAM-1 is a transmembrane cellular adhesion protein that mediates the adhesion of lymphocytes, monocytes, eosinophils, and basophils to vascular endothelium. Upregulation of VCAM-1 in endothelial cells by cytokines occurs as a result of increased gene transcription (e.g., in response to Tumor necrosis factor-alpha (TNFα) and Interleukin-1 (IL-1)). VCAM-1 is encoded by the vascular cell adhesion molecule 1 gene (VCAM1; Entrez GeneID: 7412) and is produced after differential splicing of the transcript (Genbank Accession No. NM_001078 (variant 1) or NM_080682 (variant 2)), and processing of the precursor polypeptide splice isoform (Genbank Accession No. NP_001069 (isoform a) or NP_542413 (isoform b)).

The terms “s-VCAM-1” or “serum VCAM-1” refer to the free soluble form of the vascular cell adhesion molecule 1. In certain instances, the presence or level of an s-VCAM-1 is detected at the level of mRNA expression with an assay such as, e.g., a hybridization assay or an amplification-based assay. In certain other instances, the presence or level of VCAM-1 is detected at the level of protein expression using, for example, an immunoassay (e.g., ELISA or an immuno electrochemiluminescence assay) or an immunohistochemical assay. Suitable antibodies and/or ELISA kits for determining the presence or level of VCAM-1 in a sample such as a tissue sample, biopsy, serum, plasma, saliva, urine, or stool are available from, e.g., Invitrogen (Camarillo, Calif.), Santa Cruz Biotechnology, Inc. (Santa Cruz, Calif.), and/or Abcam Inc. (Cambridge, Mass.).

7. ICAM-1

The term “ICAM-1” or “intercellular adhesion molecule 1” refers to isolated nucleic acids, polypeptides and polymorphic variants, alleles, mutants, and interspecies homologues thereof and as further described herein, that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 50, 75, 100, 150, 200, 250, 300, 400, 500, 525, or more amino acids, to a human ICAM-1 sequence shown below; (2) bind to antibodies, e.g., polyclonal antibodies, raised against an immunogen comprising an amino acid sequence shown below, or conservatively modified variants thereof; (3) bind to a ICAM-1 binding protein; (4) specifically hybridize under stringent hybridization conditions to a nucleic acid sequence shown below, or conservatively modified variants thereof; (5) have a nucleic acid sequence that has greater than about 90%, preferably greater than about 96%, 97%, 98%, 99%, or higher nucleotide sequence identity, preferably over a region of at least about 100, 200, 300, 400 or more nucleotides, to a human ICAM-1 mRNA sequence; and/or (6) have at least 25, often 50, 75, 100, 125 or 143 contiguous amino acid residues of a human ICAM-1 polypeptide sequence. The human ICAM-1 polypeptide sequence is set forth in, e.g., Genbank Accession No. NP_000192. The human ICAM-1 mRNA (coding) sequence is set forth in, e.g., Genbank Accession No. NM_000201. One skilled in the art will appreciate that variants, isoforms, alternative sequences of ICAM-1 are also useful in the present disclosure.

ICAM-1 is a transmembrane cellular adhesion protein that is continuously present in low concentrations in the membranes of leukocytes and endothelial cells. Upon cytokine stimulation, the concentrations greatly increase. ICAM-1 can be induced by IL-1 and TNFα and is expressed by the vascular endothelium, macrophages, and lymphocytes. In IBD, proinflammatory cytokines cause inflammation by upregulating expression of adhesion molecules such as ICAM-1. The increased expression of adhesion molecules recruit more lymphocytes to the infected tissue, resulting in tissue inflammation (see, Goke et al., J., Gastroenterol., 32:480 (1997); and Rijcken et al., Gut, 51:529 (2002)). ICAM-1 is encoded by the intercellular adhesion molecule 1 gene (ICAM1; Entrez GeneID: 3383; Genbank Accession No. NM_000201) and is produced after processing of the intercellular adhesion molecule 1 precursor polypeptide (Genbank Accession No. NP_000192). The terms “s-ICAM-1” or “serum ICAM-1” refer to the free soluble form of human α4β7 integrin.

8. Predicting Remittance or Non-Remittance

In some embodiments, if a UC patient has a higher or lower level of any one of the predictive biomarkers s-TNFα, s-α4β7, s-MAdCAM-1, s CRP, s AA, s-VCAM-1, s-ICAM-1 at baseline or at week 2 compared to a cut-off value, then the patient is either a remitter or a non-remitter. In some embodiments, the cut-off value for a specific predictive marker can be established from a reference population of subjects. In some cases, the reference population includes subject who had a clinical response or remission (e.g., a greater than or equal to 70-point decrease in CDAI score for CD; a reduction in the Mayo Clinical score of at least 3 points and a decrease of at least 30% from the baseline score, with a decrease of at least 1 point on the rectal bleeding score of 0 or 1 for UC) to vedolizumab.

In some embodiments, a UC patient is classified as a remitter to the anti-α4β7 integrin drug treatment (e.g., VDZ therapy) when the s-α4β7 level of the UC patient is increased. In some embodiments, a UC patient is classified as a remitter to the anti-α4β7 integrin drug treatment (e.g., VDZ therapy) when the s-TNF level of the UC patient is lower. In some embodiments, a UC patient is classified as a remitter to the anti-α4β7 integrin drug treatment (e.g., VDZ therapy) when the s-MAdCAM-1 level of the UC patient is lower. In some embodiments, a UC patient is classified as a remitter to the anti-α4β7 integrin drug treatment (e.g., VDZ therapy) when the s-ICAM-1 level of the UC patient is lower. In some embodiments, a UC patient is classified as a remitter to the anti-α4β7 integrin drug treatment (e.g., VDZ therapy) when the s-VCAM-1 level of the UC patient is lower. In some embodiments, a UC patient is classified as a remitter to the anti-α4β7 integrin drug treatment (e.g., VDZ therapy) when one or more of the s-TNF, s-MAdCAM-1, s-ICAM-1, s-VCAM-1 levels of the UC patient is lower. In some embodiments, during induction time points, s-TNF concentrations are lower in remitters. In some embodiments, during maintenance time points, s-α4β7 is higher in remitters and s-VCAM-1 is lower in remitters.

9. Setting Cut-Off Values

Once the sample(s) from the human subject have been assayed for the criteria listed above, a value is generated for predicting likelihood of clinical response or remission (e.g., as defined by the Physician's Global Assessment for CD or UC, the Crohn's Disease Activity Index, the Mayo Clinic Score, or any other standard assessment criteria or scale for IBS) to vedolizumab or likelihood of having clinical remission (e.g., as defined by the Physician's Global Assessment for CD or UC, the Crohn's Disease Activity Index, the Mayo Clinic Score, or any other standard assessment criteria or scale for IBS). When two or more predictive markers or other criteria are used in the method described herein, the level of each marker can be weighted and combined. Thus, a test value may be provided by (a) weighting the determined level of each marker with a predefined coefficient, and (b) combining the weighted level to provide a test value. The combining step can be either by straight addition or averaging (i.e., weighted equally) or by a different predefined coefficient.

Once generated, the value from a sample can be compared to one or more cut-off or threshold value(s) to provide a likelihood of clinical response or clinical remission. In order to establish a cut-off value for practicing the method, a reference population of subjects can be used. In some embodiments, a population of patients with CD or UC can be used. In some instances, the patients have had a clinical response to vedolizumab. In other instances, the patients have not had a clinical response to vedolizumab or have autoantibodies against vedolizumab. Alternatively, the patients may have a clinical response to the anti-α4β7 integrin drug. In some embodiments, the patients are in clinical remission from CD or in clinical remission from UC.

In some embodiments, these patients are within the appropriate parameters, if applicable, for the purpose of screening for and/or monitoring CD or UC using the methods of the present disclosure. Optionally, the patients are of similar age or similar ethnic background. The status of the selected patients can be confirmed by well established, routinely employed methods including but not limited to general physical examination of the individuals and general review of their medical history. Furthermore, the selected group of patients will generally be of sufficient size, such that the average value in the sample obtained from the group can be reasonably regarded as representative of a particular indication, for example indicative of reoccurrence of CD or UC or not after a set period of time (e.g., 2 years) after treatment.

Once an average value is established based on the individual values found in each subject of the selected group, this average or median or representative value or profile can be used as a cut-off value. For example, a sample value over the cut-off value can indicate a more than average likelihood of clinical response or clinical remission depending on the predictive marker used. In some embodiments, a standard deviation is also determined during the same process. In some cases, separate cut-off values may be established for separately defined groups having distinct characteristics such as age, gender, or ethnic background.

According to the methods described herein, the sample is compared to one or more reference or threshold values. In some embodiments, the sample value is deemed “high” if it is at least 1, 2, 3, 4, 5, 10, 15, 20 or more standard deviations greater than the reference value subjects. In other embodiments, the sample value is below the threshold if the sample value is at least 1, 2, 3, 4, 5, 10, 15, 20 or more standard deviations lower than the reference or threshold value.

In some embodiments, a computer-based analysis program is used to translate the raw data generated by the detection methods described herein (e.g., the presence, absence, or amount of a given marker or markers) into a score of predictive value to a clinician.

The predictive marker profile or score, as determined according to the methods above, can predict that the patient has an above-average likelihood of clinical response or remission. In some cases, the patient has a high likelihood of clinical response or remission. The score can also predict that the patient has an average or below-average likelihood of clinical response or remission. In such instances, the patient can have a low or intermediate likelihood of clinical response or remission.

D. Statistical Analysis

In some aspects, the present disclosure provides methods for selecting anti-α4137 integrin drug therapy, optimizing anti-α4β7 integrin drug therapy, reducing toxicity associated with anti-α4β7 integrin drug therapy, and/or monitoring the efficacy of anti-α4β7 integrin drug treatment by applying one or more statistical algorithm to one or more (e.g., a combination of two, three, four, five, six, seven, eight, nine, ten, eleven, twelve or more) pharmacodynamic and/or predictive markers. In particular embodiments, quantile analysis is applied to the presence and/or level of one or more markers to guide treatment decisions for patients receiving anti-α4β7 integrin drug therapy. In other embodiments, one or a combination of two of more learning statistical classifier systems are applied to the presence and/or level of one or more markers to guide treatment decisions for patients receiving anti-α4β7 integrin drug therapy. The statistical analyses of the methods of the present disclosure advantageously assist in determining when or how to adjust or modify (e.g., increase or decrease) the subsequent dose of an anti-α4β7 integrin drug, to combine an anti-α4β7 integrin drug (e.g., at an increased, decreased, or same dose) with one or more immunosuppressive agents such as methotrexate (MTX) or azathioprine (AZA), and/or to change the current course of therapy (e.g., switch to a different anti-α4β7 integrin drug).

The term “statistical analysis” or “statistical algorithm” or “statistical process” includes any of a variety of statistical methods and models used to determine relationships between variables. In the present disclosure, the variables are the presence or level of at least one marker of interest. Any number of markers can be analyzed using a statistical analysis described herein. For example, the presence or level of 1, 2, 3, 4, 5, 6, 7, 8, 9, 10, 11, 12, 13, 14, 15, 16, 17, 18, 19, 20, 25, 30, 35, 40, 45, 50, 55, 60, or more markers can be included in a statistical analysis. In one embodiment, logistic regression is used. In another embodiment, linear regression is used. In yet another embodiment, ordinary least squares regression or unconditional logistic regression is used. In certain preferred embodiments, the statistical analyses of the present disclosure comprise a quantile measurement of one or more markers, e.g., within a given population, as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present disclosure can also include the use of percentile ranges of marker levels (e.g., tertiles, quartile, quintiles, etc.), or their cumulative indices (e.g., quartile sums of marker levels to obtain quartile sum scores (QSS), etc.) as variables in the statistical analyses (just as with continuous variables).

In certain embodiments, the present disclosure involves detecting or determining the presence and/or level (e.g., magnitude) of one or more markers of interest using quartile analysis. In this type of statistical analysis, the level of a marker of interest is defined as being in the first quartile (<25%), second quartile (25-50%), third quartile (51%-<75%), or fourth quartile (75-100%) in relation to a reference database of samples. These quartiles may be assigned a quartile score of 1, 2, 3, and 4, respectively. In certain instances, a marker that is not detected in a sample is assigned a quartile score of 0 or 1, while a marker that is detected (e.g., present) in a sample (e.g., sample is positive for the marker) is assigned a quartile score of 4. In some embodiments, quartile 1 represents samples with the lowest marker levels, while quartile 4 represent samples with the highest marker levels. The reference database of samples can include a large spectrum of patients with a TNFα-mediated disease or disorder such as, e.g., IBD. From such a database, quartile cut-offs can be established. A non-limiting example of quartile analysis suitable for use in the present disclosure is described in, e.g., Mow et al., Gastroenterology, 126:414-24 (2004).

In some embodiments, the statistical analyses of the present disclosure comprise one or more learning statistical classifier systems. As used herein, the term “learning statistical classifier system” includes a machine learning algorithmic technique capable of adapting to complex data sets (e.g., panel of markers of interest) and making decisions based upon such data sets. In some embodiments, a single learning statistical classifier system such as a decision/classification tree (e.g., random forest (RF) or classification and regression tree (C&RT)) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, the Cox Proportional-Hazards Model (CPHM), perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests software available from Salford Systems (San Diego, Calif.). See, e.g., Breiman, Machine Learning, 45:5-32 (2001); and http://stat-www.berkeley.edu/users/breiman/RandomForests/cc home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the C&RT software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM^{light} software developed by Thorsten Joachims (Cornell University) or using the LIBSVM software developed by Chih-Chung Chang and Chih-Jen Lin (National Taiwan University).

The various statistical methods and models described herein can be trained and tested using a cohort of samples (e.g., serological and/or genomic samples) from healthy individuals and patients with a TNFα-mediated disease or disorder such as, e.g., IBD (e.g., CD and/or UC) or rheumatoid arthritis. For example, samples from patients diagnosed by a physician, preferably by a gastroenterologist, as having IBD or a clinical subtype thereof using a biopsy, colonoscopy, or an immunoassay as described in, e.g., U.S. Pat. No. 6,218,129, are suitable for use in training and testing the statistical methods and models of the present disclosure. Samples from patients diagnosed with IBD can also be stratified into Crohn's disease or ulcerative colitis using an immunoassay as described in, e.g., U.S. Pat. Nos. 5,750,355 and 5,830,675. Samples from healthy individuals can include those that were not identified as IBD samples. One skilled in the art will know of additional techniques and diagnostic criteria for obtaining a cohort of patient samples that can be used in training and testing the statistical methods and models of the present disclosure.

E. Predictive Modeling

In certain aspects, the present disclosure provides pharmacokinetic models to predict the likelihood of developing anti-drug antibodies.

Pharmacokinetic models are ways to mathematically understand the fate of drugs in vivo. In a one compartment model, the drug-concentration time profile shows a monophasic response, and is described by a single exponential. In addition, the body is assumed to be a homogeneous unit with instantaneous distribution of the drug. A one-compartment model shows a linear relationship between log concentrations in plasma (Cₚ) versus time.

A two-compartment model resolves the body into two units, a central unit and a peripheral unit. In the two-compartment model, the log concentration in plasma (Cₚ) versus time profile is biphasic. In the biphasic model, there is a rapid decline in drug concentration followed by a slower decline. D. Ternant et al., Ther Drug Monit, 30(4), 523-529 (2008), showed that infliximab pharmacokinetics followed a two compartment model, with an elimination half-life of close to 3 weeks.

In other aspects, the present disclosure provides an algorithmic model to predict patient response to anti-α4β7 integrin drug. The model uses one or more markers such as an inflammatory marker which include cytokines and chemokines and the like, a signaling molecule, an acute phase protein, a cellular adhesion molecule and a combination thereof. The markers also include the presence or absence of ADA, the levels of α4β7 integrin, the levels of MAdCAM-1, the concentration or levels of anti-α4β7 integrin drugs and the like.

An algorithmic model includes any of a variety of statistical methods and models used to determine relationships between variables. In the present disclosure, the variables are the presence or level of at least one marker of interest. Any number of markers can be analyzed using a statistical analysis described herein (see, “Statistical Analysis” section). For example, the presence or level of 1, 2, 3, 4, 5, 6, 7, 8, 9, 10, 11, 12, 13, 14, 15, 16, 17, 18, 19, 20, 25, or more markers can be included in a statistical analysis.

In particular embodiments, quantile analysis is applied to the presence and/or level of one or more markers to guide treatment decisions for patients receiving anti-α4β7 integrin drug therapy. In other embodiments, one or a combination of two of more learning statistical classifier systems are applied to the presence and/or level of one or more markers to guide treatment decisions for patients receiving anti-α4β7 integrin drug therapy. The statistical analyses of the methods of the present disclosure advantageously assist in determining when or how to adjust or modify (e.g., increase or decrease) the subsequent dose of an anti-α4β7 integrin drug, to combine an anti-α4β7 integrin drug (e.g., at an increased, decreased, or same dose) with one or more immunosuppressive agents such as methotrexate (MTX) or azathioprine (AZA), and/or to change the current course of therapy (e.g., switch to a different anti-α4β7 integrin drug).

The algorithmic model includes the level or concentration of the one or more markers along with a statistic algorithm such as a learning statistical algorithm. In certain instances, the model has been trained with known outcomes using a training set cohort of samples. The algorithm is then validated using a validation cohort. Patient unknown samples can then be predicted based on the trained algorithms.

In some aspects, the present disclosure provides a system for predicting the level of an anti-α4β7 integrin drug in a subject at a later time point during a course of therapy with the anti-α4β7 integrin drug. In other aspects, the present disclosure provides a system for predicting whether a subject will develop autoantibodies to an anti-α4β7 integrin drug at a later time point during a course of therapy with the anti-α4β7 integrin drug. In yet other aspects, the present disclosure provides a system for predicting a clinical outcome of a subject at a later time point during a course of therapy with the anti-α4β7 integrin drug.

In certain embodiments, the system comprises: a data acquisition module configured to produce a data set comprising one or more predictor variables for the subject determined at an earlier time point during the course of therapy and/or prior to the initiation of the course of therapy; a data processing module configured to process the data set by applying a statistical analysis to the data set to produce a statistically derived decision predicting the level of the anti-α4β7 integrin drug or predicting whether the subject will develop autoantibodies to the anti-α4β7 integrin drug or predicting a clinical outcome of the subject receiving the anti-α4β7 integrin drug based upon the one or more predictor variables; and a display module configured to display the statistically derived decision.

In some embodiments, the system includes an intelligence module, such as a computer, having a processor and memory module. The intelligence module may also include communication modules for transmitting and receiving information over one or more direct connections (e.g., USB, Firewire, or other interface) and one or more network connections (e.g., including a modem or other network interface device). The memory module may include internal memory devices and one or more external memory devices. The intelligence module also includes a display module, such as a monitor, screen, or printer. In one aspect, the intelligence module receives data such as patient test results from a data acquisition module such as a test system, either through a direct connection or over a network. For example, the test system may be configured to run multianalyte tests on one or more patient samples and automatically provide the test results to the intelligence module. The data may also be provided to the intelligence module via direct input by a user or it may be downloaded from a portable medium such as a compact disk (CD) or a digital versatile disk (DVD). The test system may be integrated with the intelligence module, directly coupled to the intelligence module, or it may be remotely coupled with the intelligence module over the network. The intelligence module may also communicate data to and from one or more client systems over the network as is well known. For example, a requesting physician or healthcare provider may obtain and view a report from the intelligence module, which may be resident in a laboratory or hospital, using a client system.

The network can be a LAN (local area network), WAN (wide area network), wireless network, point-to-point network, star network, token ring network, hub network, or other configuration. As the most common type of network in current use is a TCP/IP (Transfer Control Protocol and Internet Protocol) network such as the global internetwork of networks often referred to as the “Internet” with a capital “I,” that will be used in many of the examples herein, but it should be understood that the networks that the present disclosure might use are not so limited, although TCP/IP is the currently preferred protocol.

Several elements in the system may include conventional, well-known elements that need not be explained in detail here. For example, the intelligence module could be implemented as a desktop personal computer, workstation, mainframe, laptop, etc. Each client system could include a desktop personal computer, workstation, laptop, cell phone, tablet, PDA, or any WAP-enabled device or any other computing device capable of interfacing directly or indirectly to the Internet or other network connection. A client system typically runs an HTTP client, e.g., a browsing program, such as Microsoft's Internet Explorer™ browser, Google's Chrome™ browser, or a WAP-enabled browser or mobile application in the case of a cell phone, tablet, PDA, or other wireless device, or the like, allowing a user of the client system to access, process, and view information and pages available to it from the intelligence module over the network. Each client system also typically includes one or more user interface devices, such as a keyboard, a mouse, touch screen, pen, or the like, for interacting with a graphical user interface (GUI) provided by the browser on a display (e.g., monitor screen, cell phone or tablet screen, LCD display, etc.) in conjunction with pages, forms, and other information provided by the intelligence module. As discussed above, the present disclosure is suitable for use with the Internet, which refers to a specific global internetwork of networks. However, it should be understood that other networks can be used instead of the Internet, such as an intranet, an extranet, a virtual private network (VPN), a non-TCP/IP based network, any LAN or WAN, or the like.

According to one embodiment, each client system and all of its components are operator configurable using applications, such as a browser, including computer code run using a central processing unit such as an Intel® Pentium® processor or the like. Similarly, the intelligence module and all of its components might be operator configurable using application(s) including computer code run using a central processing unit such as an Intel® Pentium® processor or the like, or multiple processor units. Computer code for operating and configuring the intelligence module to process data and test results as described herein is preferably downloaded and stored on a hard disk, but the entire program code, or portions thereof, may also be stored in any other volatile or non-volatile memory medium or device as is well known, such as a ROM or RAM, or provided on any other computer readable medium capable of storing program code, such as a compact disk (CD) medium, digital versatile disk (DVD) medium, a floppy disk, ROM, RAM, and the like.

The computer code for implementing various aspects and embodiments of the present disclosure can be implemented in any programming language that can be executed on a computer system such as, for example, in C, C++, C#, HTML, Java, JavaScript, or any other scripting language, such as VB Script. Additionally, the entire program code, or portions thereof, may be embodied as a carrier signal, which may be transmitted and downloaded from a software source (e.g., server) over the Internet, or over any other conventional network connection as is well known (e.g., extranet, VPN, LAN, etc.) using any communication medium and protocols (e.g., TCP/IP, HTTP, HTTPS, Ethernet, etc.) as are well known.

### III. Examples

### Example 1: Methods for Monitoring Vedolizumab Levels in Patients with Inflammatory Bowel Diseases as a Function of Time and Assessing Remission in Patients Thereof

The aim of this example was to determine the relationship between serum vedolizumab trough levels (VTL) during induction and disease remission after 22 weeks of therapy. Antibodies against vedolizumab (ATV) were also monitored.

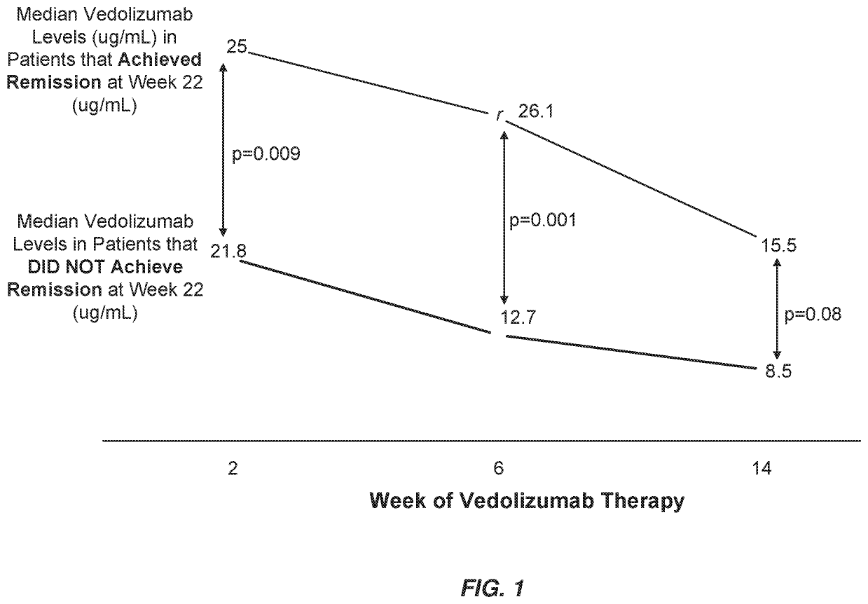

Samples were obtained from 45 patients with IBD with active diseases starting vedolizumab. The cross-sectional study included patients with Crohn's disease (CD) and patients with ulcerative colitis (UC). Concentrations of vedolizumab and ATV were measured by homogenous mobility shift assay (HMSA). In general, VTL at weeks 2, 6 and 14 were higher in the patients that achieved remission by week 22 compared to the patients that did not achieve remission at week 22 (FIG. 1), with significant statistical differences between the two groups at weeks 2 and 6.

Patients with a VTL ≥24 ug/mL at week 2 and ≥10.6 were more likely to be in remission at week 22 (OR: 5 [95% CI:1.4-17.8], p:0.001 and OR: 13.5 [95% CI: 1.5-118.7], p=0.005 respectively). Three patients developed ATV during induction. The antibodies were undetectable by week 14 in all patients. The association between levels and other variables with remission are shown below in Table 1.

TABLE 1
Levels of VDZ during remission

| | | | Steroid free | |
|---|---|---|---|---|
| | Remission week 22 | | remission week 22 | |
| Variable | [ROC (p value)] | | [ROC (p value)] | |
| Vedolizumab Level Week 2 | 0.73 | (0.048*) | 0.63 | (0.26) |
| Vedolizumab Level Week 6 | 0.86 | (0.003*) | 0.77 | (0.004*) |
| Vedolizumab Level Week 14 | 0.68 | (0.09) | 0.57 | (0.47) |
| Age | 0.53 | (0.99) | 0.5 | (0.65) |
| Baseline CRP | 0.61 | (0.57) | 0.51 | (0.94) |
| Baseline Albumin | 0.832 | (0.0007*) | 0.76 | (<0.001*) |
| Body Mass | 0.55 | (0.61) | 0.5 | (0.99) |

*Statistically significant

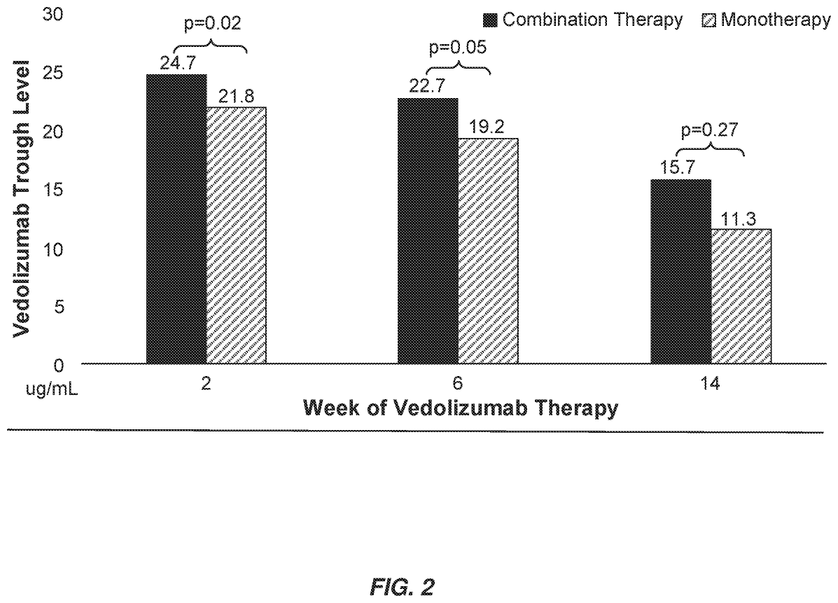

By week 22, the patients having that had achieved deep remission showed normal C-reactive protein levels. Those patients in remission from CD achieved a Simple Endoscopic Score for Crohn's Disease of ≤2, while patients in remission from in UC achieved a Mayo Endoscopic Score of ≤1. Another primary outcome of IBD patients having received vedolizumab treatment for 22 weeks is clinical remission, in which the Harvey-Bradshaw Index score for CD is <5 and the Mayo Clinical Score for UC is <3. Steroid-free remission by week 22 is a secondary outcome, in which the patient has achieved deep remission while off steroids for 3 months. As shown in FIG. 2, VTL were higher in patients who received combination therapy. However, only the data for week 2 is considered statistically significant.

Higher vedolizumab levels during induction are associated with better response to therapy at week 22. Therapeutic drug monitoring (TDM) may have a role in patients with CD or UC receiving vedolizumab and early optimization may potentially be of benefit in these patients.

### Example 2: Association of Vedolizumab Concentrations Using the ANSER® VDZ Mobility Shift Assay and Clinical Response in IBD Patients in Clinical Practice

Vedolizumab (VDZ), an α4β7 integrin antagonist, is a novel therapeutic agent approved for treatment of moderate to severe ulcerative colitis (UC) and Crohn's disease (CD). Availability of a drug-tolerant assay to measure serum VDZ levels and antibodies to VDZ (ATV) titers is important for the effective use of this drug in clinical practice. The aim of this study was to measure VDZ and ATV concentrations in serum using the ANSER VDZ mobility shift assay and to assess the clinical utility of the assay.

This was a real world cohort of IBD patients treated with vedolizumab at a tertiary IBD center where the majority of patients had failed TNFα inhibitors, and most patients were steroid dependent. The median follow up was 48 weeks. The median serum VDZ levels were determined as 20.4, 18, 11.3 and 10.1 μg/mL at weeks 2, 6, 14 and 26-32, respectively.

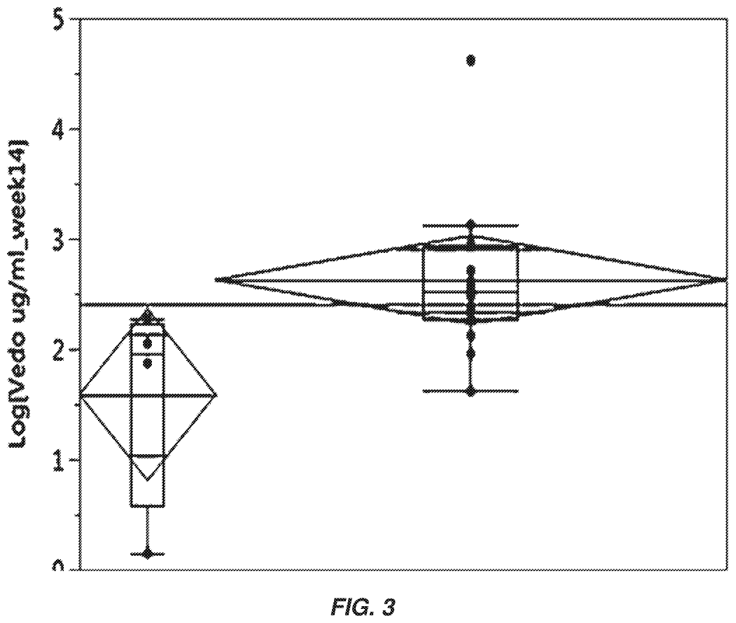

As shown in FIG. 3, serum VDZ concentration at the end of the induction at week 14 were higher in clinical responders versus non responders at the time of last follow up (see, Table 2). Median concentrations of inflammatory markers (SAA-1 and TNFα) were lower in clinical responders to VDZ versus non-responders; however, CRP was not significantly different. Approximately 11% (4/35) patients developed ATV (3 UC and 1 CD). ATV formation was detected early in treatment during the induction phase. ATV were detected despite the presence of concomitant VDZ levels as high as 26 mg/ml.

TABLE 2
Clinical responders versus non responders

| PGA Response | Non-Responder | Responder |
|---|---|---|
| N | 4 | 15 |
| Median VDZ (μg/mL) | 7.11 | 12.48 |

p = 0.02*

### Example 3: Association of Vedolizumab Concentrations During Induction with Long-Term Clinical and Endoscopic Remission in IBD Patients

The following example demonstrates the relationship of serum vedolizumab (VDZ) concentrations during induction and long term endoscopic outcomes and disease remission in patients with inflammatory bowel diseases (IBD) after 52 weeks of therapy. This example also assessed the incidence, prevalence, and clinical significance of the formation of detectable antibodies to VDZ (ATV). Variables associated with serum VDZ concentrations through the first 30 weeks of treatment were identified and a correlation between serum VDZ concentrations and serological markers with disease activity was assessed.

### Methods

### Subjects and Setting

A prospective cohort study was performed in patients with CD or UC with moderate to severe active endoscopic disease, and who were initiating VDZ therapy at the Medical College of Wisconsin/Froedtert Hospital (Milwaukee, Wis.). The study was approved by the local Institutional Review Board and every patient signed an informed consent. Patients were included if they had active endoscopic disease and were starting therapy with VDZ. Active endoscopic disease was defined as a simple endoscopic score for Crohn's disease (SES-CD) ≥2 (in CD patients) or an endoscopic Mayo score ≥1 (in UC patients). See, Daperno M. et al. Gastrointest. Endosc. 2004, 60, 505-512; Schroeder K. W. et al. N Engl Med 1987, 317, 1625-1629.

All patients received induction with VDZ, 300 mg at weeks 0, 2 and 6 followed by 300 mg every 8 weeks. At the time a patient was enrolled, a complete history and assessment of disease was completed. Patients with ostomies, patients with previous exposure to VDZ, and patients with no evidence of baseline active endoscopic disease were excluded from the study. Those patients that were lost to follow-up or discontinued VDZ for reasons other than lack of efficacy (such as intolerance or lack of insurance coverage) were also excluded from the study.

### Evaluations and Input Variables

Independent variables considered were demographics, IBD phenotype, smoking status, body weight, previous exposure to biologics and concomitant medications used for the treatment of IBD. Assessment of endoscopic disease activity was considered at baseline (before starting VDZ) and at the time of primary outcome (week 52 [+/−8 weeks]). Endoscopic assessment was also recorded at week 52 (+/−8 weeks). The endoscopist performing the colonoscopies and clinicians caring for each patient were blinded to the serum drug concentrations and antibodies for each patient.

Clinical disease activity was also recorded at baseline and at the time each serum sample was drawn (just prior to each infusion). The Harvey Bradshaw Index (HBI) was used to assess clinical disease activity in patients with CD. See, Harvey R F, Bradshaw J M. Lancet 1980, 1, 514. A score <5 was considered as remission. The partial Mayo score was used to assess disease in those patients with UC. See, Lewis J. D. et al. Inflamm Bowel Dis 2008, 14, 1660-1666. A score <2 was considered remission.

Serum samples were drawn just before the VDZ infusion at weeks 2, 6, 14, 22 and 30 for measurement of serum vedolizumab levels (SVL) and ATV (unless the drug was discontinued or the patient lost follow-up). C-reactive protein (CRP) and albumin levels were also measured at weeks 0, 14, 30 and 52. Fecal calprotectin levels were recorded at baseline. Normal serum CRP and albumin levels, defined as per the used assay, were <0.5 mg/dL and ≥3.5 mg/dL, respectively.

Disease phenotype was classified according to the Montreal classification. See, Silverberg M. S. et al. Can J Gastroenterol 2005, 19, Suppl A: 5-36. CD was categorized as ileal, colonic, or ileo-colonic, with or without upper gastrointestinal tract involvement and stricturing or fistulizing disease. UC was classified as proctitis, left-sided disease, or extensive involvement (pan-colitis).

Use of IBD medications was recorded. 5-aminosalicylates were considered if prescribed for 30 or more consecutive days prior to the initiation of VDZ. Corticosteroid (CS), including budesonide, were recorded at the time VDZ was started and at each follow up. Rectally administered topical steroids (e.g., enemas or suppositories) were not considered in the analysis. Immunomodulators (azathioprine, 6-mercaptopurine, and methotrexate) were also considered if started concomitantly with VDZ or if the patient had been on it before starting. Previous exposure to biologics and response to therapy was recorded at baseline for every patient. Changes in medications (including corticosteroids) were assessed. The VDZ dose escalation was also recorded, which was performed as standard of care by the treating provider. VDZ dose escalation was defined as decreasing the interval of drug administration to every 4 weeks. Clinicians managing each patient were blinded to the drug levels and dose escalation or drug discontinuation was not influenced by pharmacokinetic data.

### Measurement of VDZ Levels and Anti-VDZ Antibodies

Serum levels of VDZ and ATV were measured using a validated drug-tolerant assay specific for VDZ that utilized homogeneous mobility shift assay (HMSA) methodology. See, Wang, S-L et al. Journal of Immunological Methods 2012, 382, 177-188. Briefly, VDZ levels were measured by calculating the shift in antigen bound VDZ complex on a size exclusion column by HPLC. ATV levels were quantified using a size exclusion chromatography-based mobility shift assay, run on a HPLC system with fluorescent detection. The lower limits of quantitation for VDZ and ATV were 1.6 μg/mL and 1.6 U/mL, respectively. The upper limits of quantitation for VDZ and ATV were 40 μg/mL and 75 U/mL, respectively. The investigators conducting the measurements were blinded to the demographic, clinical and histologic data of the patients.

### Outcomes

The primary outcome was steroid-free endoscopic remission at week 52 while on a standard dose of VDZ (+/−8 weeks). Endoscopic remission was defined as an SES-CD<2 in patients with CD and an endoscopic mayor score (EMS)<2 in UC patients while off corticosteroids. Patients that discontinued the drug due to lack of efficacy and/or required dose escalation were considered to have failed the primary outcome.

Secondary outcomes were steroid-free clinical remission at weeks 14, 22, 30 and 52, drug discontinuation (stratified as early [within 6 months of drug initiation] and late [after 6 months of drug initiation]) and need for drug dose escalation. Clinical remission was defined as an HBI <5 in CD and a partial Mayo score (pMS)<2 in UC. Loss of response was defined as having achieved clinical remission at week 22 and then experiencing disease recurrence at any time point through week 52 (HBI 5/pMS need for new course of steroids due to disease exacerbation, and/or discontinuation of the drug due to inefficacy).

### Statistical Analysis

Descriptive statistics were used to examine the baseline characteristics of the study population. Continuous variables were compared using Student's t-test or the Mann-Whitney U-test (for nonparametric variables). The x² test was used to evaluate distributions of categorical variables. Correlation between serum VDZ concentrations and other continuous variables were performed using Spearman's rank correlation test. Receiver operating characteristic curves (ROC) were generated in order to look for association of outcomes and VDZ levels. A p value <0.05 was considered statistically significant.

### Results

### Patient Characteristics and Outcomes

Fifty-five patients met inclusion criteria and were followed for a median of 44 weeks (range of 8-54 weeks). A total of 244 samples were analyzed for those 55 patients (average of 4.4 samples per patient). The baseline characteristics of the study group at the time of VDZ initiation are shown in Table 3. Twenty-six patients (47%) achieved the primary outcome of steroid free endoscopic remission at week 52 of therapy (56% of those with CD and 40% of those with UC). Thirty (54.6%), 32 (58.2%) and 27 (49%) of patients were in clinical remission at week 14, 22, and 30 respectively. Twenty-three (42%) had discontinued the drug by the end of follow up (week 52). Eight (35%) of those patients that discontinued VDZ did so before week 30 of therapy. Ten patients (18%) were dose escalated to 300 mg of VDZ every 4 weeks at some point through the follow-up (between weeks 14 and 52).

TABLE 3
Baseline characteristics of the population

| | | |
|---|---|---|
| Age [mean in years (SD)] | 42 | (17) |
| Female gender [n (%)] | 14 | (25.5) |
| Body mass [mean in Kilograms (SD)] | 83.9 | (20.7) |
| Diagnosis of Crohn's disease | 25 | (45.5) |
| (Vs. ulcerative colitis) [n (%)] | | |
| Duration of IBD [median in years (IQR)] | 10 | (4-16) |
| Active smoking [n (%)] | 4 | (7.3) |
| Receiving aminosalicylates [n (%)] | 2 | (3.6) |
| On steroids when starting vedolizumab [n (%)] | 36 | (32.7) |
| Naïve to biologies [n (%)] | 18 | (33) |
| Receiving combination therapy with an | 20 | (36.4) |
| immunomodulator [n (%)] | | |
| On a thiopurine [n (%)] | 17 | (31) |
| On methotrexate [n (%)] | 3 | (5.5) |

Crohn's disease phenotype and disease activity¹

| | | |
|---|---|---|
| Ileal [n (%)] | 10 | (40) |
| Ileo-colonic [n (%)] | 9 | (36) |
| Colonic disease [n (%)] | 6 | (24) |
| Fistulizing disease [n (%)] | 6 | (24) |
| Stricturing phenotype [n (%)] | 12 | (48) |
| Baseline Simple Endoscopic Score - CD | | |
| [median score (IQR)] | | |
| Baseline HBI [median score (IQR)] | 3 | (1.3-6.8) |

Ulcerative colitis phenotype and disease activity²

| Proctitis [n (%)] | None | |
|---|---|---|
| Left colitis [n (%)] | 9 | (30) |
| Pan-colitis [n (%)] | 21 | (70) |
| Baseline Mayo score [median score (IQR)] | 5 | (3.8-6.3) |

Baseline laboratory data

| | | |
|---|---|---|
| Fecal calprotectin [median in ug/g feces (IQR)] | 271 | (120.8-410.8) |
| Albumin [mean in gr/dL (SD]) | 4.1 | (3.8-4.4) |
| C-reactive protein [median in mg/dL (IQR)] | 0.4 | (0.1-0.9) |

¹Percentage of patients with Crohn's disease;
²Percentage of patients with ulcerative colitis

### VDZ Levels, Anti-Drug Antibodies, and Outcomes

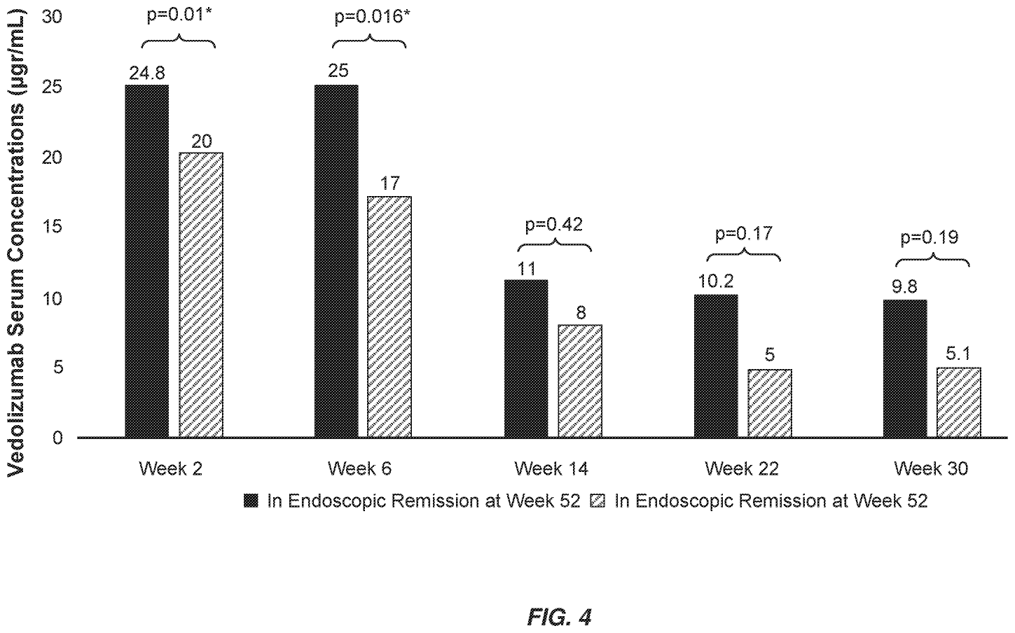

Median serum VDZ concentrations were 23.1 mcg/ml at week 2 (IQR: 19.4-25.8), 19.8 mcg/ml at week 6 (IQR: 12.4-26.4), 9.2 mcg/ml at week 14 (IQR:6-16), 6.9 mcg/ml at week 22 (IQR:3.6-12) and 5.8 mcg/ml at week 30 (IQR: 4.1-11). Patients that achieved steroid-free endoscopic remission by week 52 had higher serum VDZ concentrations at weeks 2, 6, 14, 22 and 30, but only achieved statistical significance at weeks 2 and 6 (FIG. 4; Table 4). Patients in clinical remission at week 52 had significantly higher median serum VDZ concentrations at week 2 when compared to those that were not (23.8 [IQR: 19-32] Vs. 19.8 [IQR: 15-23] mcg/ml, p<0.01).

TABLE 4
Baseline characteristics of cohorts on VDZ in endoscopic remission
at week 52 and not in endoscopic remission at week 52

| Variable | In remission | | Not in remission | | p value |
|---|---|---|---|---|---|
| Age [mean in years (SD)] | 39 | (15) | 45 | (19) | 0.18 |
| Diagnosis of CD (vs. UC) [n (%)] | 14 | (54) | 11 | (38) | 0.24 |
| Body mass [mean in Kg (SD)] | 83 | (17) | 84.7 | (24) | 0.77 |
| Time with IBD [mean in years (SD)] | 11 | (4-16) | 10 | (4-15) | 0.87 |
| VDZ week 2 [median in mcg/ml (IQR)] | 24.8 | (23-28) | 20 | (18-25) | 0.005* |
| VDZ week 6 [median in mcg/ml (IQR)] | 25 | (17-28) | 17.3 | (10-24) | 0.016* |
| VDZ week 14 [median in mcg/ml (IQR)] | 11 | (7-17) | 8 | (6-14) | 0.42 |
| VDZ week 22 [median in mcg/ml (IQR)] | 10.2 | (4.5-14) | 5 | (3.4-10) | 0.12 |
| VDZ week 30 [median in mcg/ml (IQR)] | 9.8 | (5.2-13) | 5.1 | (3.3-10.6) | 0.19 |
| HBI [median score (IQR)]¹ | 2.5 | (1-5) | 5.5 | (2-12) | 0.28 |
| Mayo score [median score (IQR)]² | 5 | (3-7) | 5.5 | (5-6.25) | 0.45 |
| Fecal calprotectin [median in ug/g feces (IQR)] | 204 | (81-391) | 324 | (169-411) | 0.15 |
| Albumin [mean in gr/dL (SD]) | 4.2 | (4-4.6) | 3.8 | (3.7-4.2) | 0.013* |
| C-reactive protein [median in mg/dL (IQR)] | 0.3 | (0.1-0.7) | 0.7 | (0.3-1.2) | 0.09 |
| SES-CD score [median score (IQR)]¹ | 5 | (4-6.5) | 14 | (8-22) | 0.02* |
| Endoscopic Mayo Score [median score (IQR)]² | 2 | (2-3) | 2.5 | (2-3) | 0.24 |
| UCEIS [median score (IQR)]² | 4 | (2-5) | 5 | (4-7) | 0.15 |

¹Patients with Crohn's disease;
²Patients with ulcerative colitis;
*Statistically significant

Those who achieved clinical remission at week 30 demonstrated higher serum VDZ concentrations at weeks 2, 6 and 14, with only weeks 2 and 6 reaching statistical significance (Table 4, p=0.005 and p=0.016, respectively). There were no significant differences in serum VDZ concentrations throughout induction between those patients that were and were not escalated during maintenance. When comparing serum VDZ concentrations between patients with UC and CD, there was an overall trend towards higher serum VDZ concentrations in those patients with CD vs. those with UC, even though the difference only reached statistical significance at week 2 (Table 5, CD: 24.2 [IQR: 21.7-29.3]; UC: 22.5 [IQR: 17.6-25.3]; p=0.04).

TABLE 5
Serum VDZ levels in CD patients and UC patients during therapy

| | CD | UC | p value |
|---|---|---|---|
| VDZ week 2 [median | 24.2 (21.7-29.3) | 22.5 (17.6-25.3) | 0.04* |
| in mcg/ml (IQR)] | | | |
| VDZ week 6 [median | 22.8 (16.2-25.8) | 17.4 (11.4-28.3) | 0.5 |
| in mcg/ml (IQR)] | | | |
| VDZ week 14 [median | 10.2 (6-16.2) | 8.6 (5.9-16.1) | 0.83 |
| in mcg/ml (IQR)] | | | |
| VDZ week 22 [median | 7.5 (3.7-13.2) | 6.2 (3.6-11.7) | 0.7 |
| in mcg/ml (IQR)] | | | |
| VDZ week 30 [median | 6.8 (3.9-11) | 5.8 (3.9-17.7) | 0.33 |
| in mcg/ml (IQR)] | | | |

*Statistically significant

Area under the curve measurements showed good correlation between serum VDZ concentrations at weeks 2 and 6, and endoscopic remission at weeks 52 (ROC: 0.72 [p=0.02] and ROC: 0.69 [p=0.023]). Good correlation between serum VDZ concentrations at week 6 and clinical remission at week 14 (ROC: 0.7 [p=0.02]) was also demonstrated. Furthermore, there was a significant association between serum VDZ concentrations at week 2 and clinical remission at weeks 30 and week 52 (ROC: 0.73 [p=0.015] and ROC: 0.72 [p=0.014]). Three of the 55 study subjects (5.5%) had detectable anti-vedolizumab antibodies at some point through the follow-up. One had detectable antibodies at week 2 that became undetectable at week 6, with no recurrence at week 14 (VDZ was discontinued due to no response at week 20). The two other patients had detectable antibodies at week 2 that became undetectable at week 6. Neither of these patients were in remission at week 52.

### VDZ Drug Concentrations and Loss of Response

Five patients loss clinical response throughout follow-up. Only one of those patients was in endoscopic remission despite the presence of symptoms. Those patients that had achieved clinical remission at week 22 but lost clinical response by week 52 has significantly lower median VDZ concentrations at week 14 when compared to those that did not lose response (6 [IQR: 3-8.8] and 11.2 [IQR: 5.7-16.7] respectively, p=0.02). The difference in VDZ concentrations at other time points was not statistically significant. However, those patients that lost response had numerically lower concentration levels throughout the maintenance phase of therapy (Table 6).

TABLE 6
Serum VDZ levels in patients with loss
of therapy response¹ and in remission

| | Loss of response | In remission | p value |
|---|---|---|---|
| VDZ week 2 [median | 25 (16.1-27) | 23.9 (20.4-26.5) | 0.79 |
| in mcg/ml (IQR)] | | | |
| VDZ week 6 [median | 22.8 (16.2-25.8) | 24.5 (18.1-27.3) | 0.42 |
| in mcg/ml (IQR)] | | | |
| VDZ week 14 [median | 6 (2.9-8.8) | 11.2 (5.7-16.7) | 0.02* |
| in mcg/ml (IQR)] | | | |
| VDZ week 22 [median | 4.1 (0-7.6) | 8.8 (3.7-11.8) | 0.1 |
| in mcg/ml (IQR)] | | | |
| VDZ week 30 [median | 3.3 (2.5-10) | 7.1 (4.6-10.6) | 0.22 |
| in mcg/ml (IQR)] | | | |

¹“loss of therapy response” refers to a patient in clinical remission at week 22 and developing symptoms by week 52;
*Statistically significant

### Biomarkers, Body Mass, and VDZ Pharmacokinetics

Overall, there was a negative correlation between baseline (week 0) C-reactive protein (CRP) and serum VDZ concentrations throughout the study period, even though only the levels at week 14 achieved statistical significance (Table 7). There was a positive correlation between baseline serum albumin and serum VDZ concentrations at every time point except for week 2 (Table 7). There was also an inverse correlation between baseline fecal calprotectin (FC) levels and serum VDZ concentrations, even though it only reached statistical significance at week 2 (rho: −0.43, p=0.002) and week 6 (rho: −0.31, p=0.03).

TABLE 7
Correlation between serum VDZ levels
and baseline¹ biomarker levels

| | CRP² | | Serum albumin | | FC³ | |
|---|---|---|---|---|---|---|
| VDZ week 2 | −0.27 | (0.05) | 0.23 | (0.1) | −0.43 | (0.002*) |
| [rho (p value)] | | | | | | |
| VDZ week 6 | −0.23 | (0.09) | 0.39 | (0.003*) | −0.31 | (0.3*) |
| [rho (p value)] | | | | | | |
| VDZ week 14 | −0.28 | (0.042*) | 0.3 | (0.027*) | −0.26 | (0.08) |
| [rho (p value)] | | | | | | |
| VDZ week 22 | −0.22 | (0.17) | 0.33 | (0.031*) | −0.27 | (0.113) |
| [rho (p value)] | | | | | | |
| VDZ week 30 | −0.16 | (0.43) | 0.46 | (0.02*) | −0.41 | (0.05) |
| [rho (p value)] | | | | | | |

¹week 0;
²C-reactive protein;
³fecal calprotectin;
*Statistically significant

There was an inverse correlation between body mass and serum VDZ concentrations at weeks 2 (rho: −0.34, p=0.01), week 6 (rho: −0.24, p=0.08), week 14 (rho: −0.12, p=0.39), week 22 (rho: −0.16, p=0.31), and week 30 (rho: −0.25, p=0.21), reaching statistical significance at week 2. Moreover, no correlation between age and serum VDZ concentrations was observed at any time point (p≥0.05 for all). Furthermore, the combination of baseline albumin and CRP level was associated with endoscopic remission at week 52 (ROC: 0.72, p=0.0139). This association increased to a ROC of 0.79 (p=0.002) when adding VDZ concentration at week 2 into the predictive model. The inclusion of other variables in the model (e.g., fecal calprotectin and body mass) did not increase the performance.

### Combination Therapy, Thiopurine Metabolites, and VDZ Levels

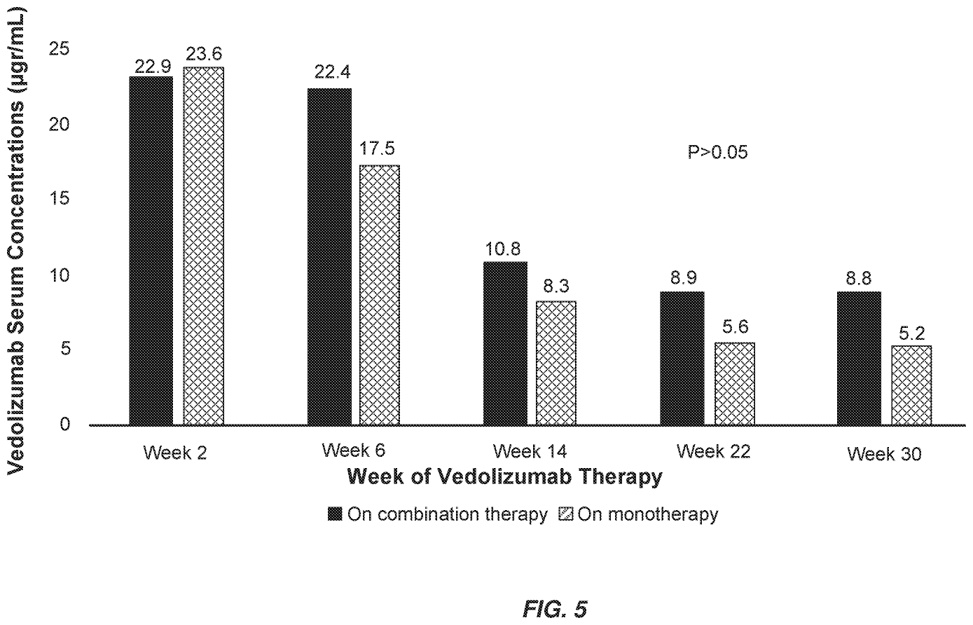

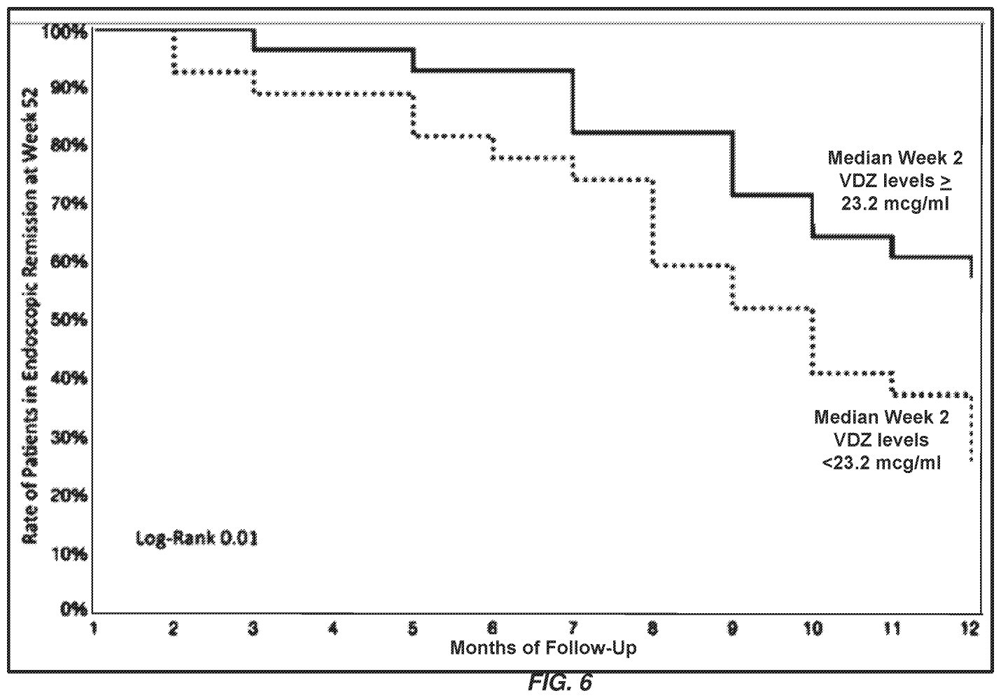

Prior exposure to biologic medications and concurrent immunomodulator use did not impact serum VDZ concentrations. Twenty patients were on combination therapy (3 on methotrexate and 17 on a thiopurine). Patients on combination therapy with an immunomodulator did not have notable differences in serum VDZ concentrations at weeks 2, 6, 14, 22, or 30 (FIG. 5). Even though the analysis was limited due to the low number of patients on azathioprine or mercaptopurine, there was no significant correlation between thiopurine drug dose and serum VDZ concentrations at week 2 (rho: −0.05 [p=0.843]), week 6 (rho: −0.024 [p=0.92]), week 14 (rho: 0.32 [p=0.19]), week 22 (rho: 0.5 [p=0.14]) or week 30 (rho: 0.37 [p=0.3]). Furthermore, there was no correlation between 6-thioguanine levels measured between weeks 14 and 30 and serum VDZ concentrations at any time point (p≥0.05 for all).

### VDZ Interquartile Thresholds During Induction and Disease Remission at Week 52

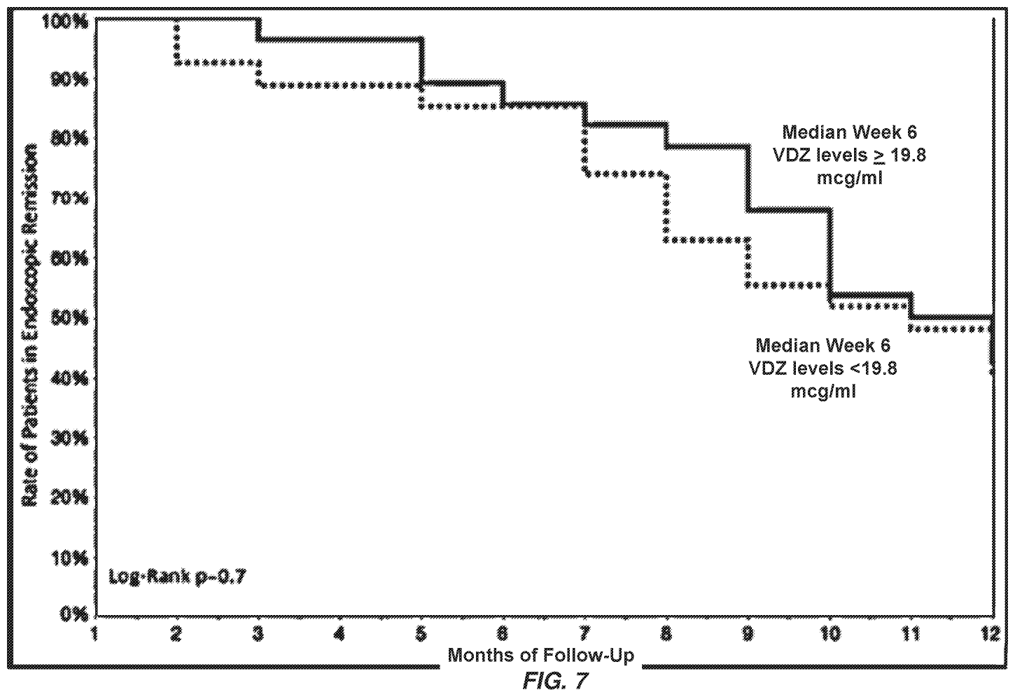

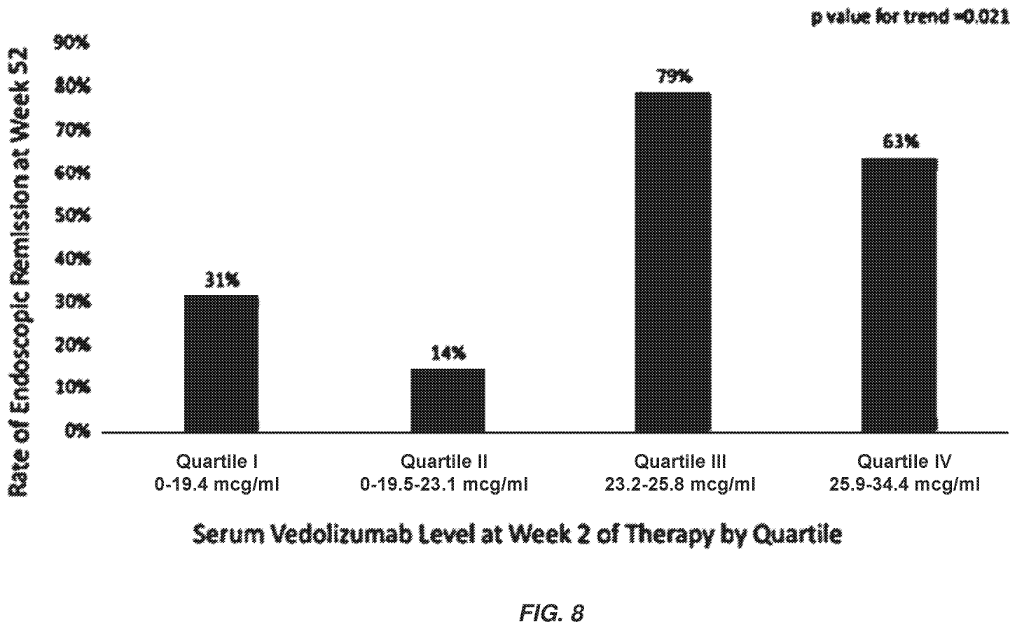

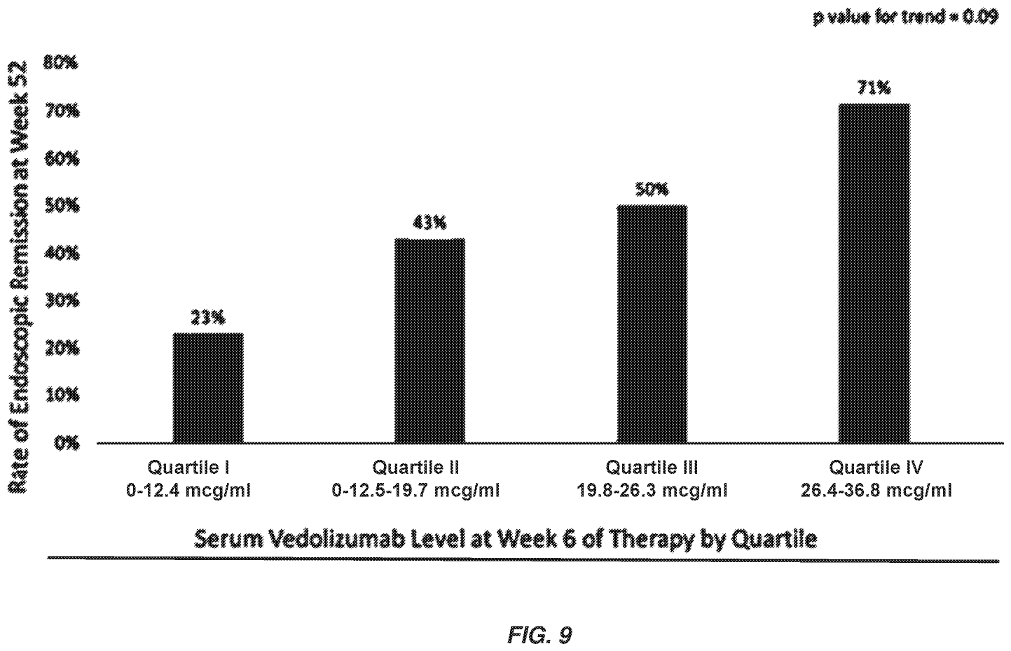

After identifying the association between disease remission and serum VDZ concentrations at weeks 2 and 6, threshold levels that best predicted disease remission at those time points were then investigated. Interquartile values for serum VDZ concentrations at week 2 were 19.4, 23.1 and 25.8 mcg/ml (FIG. 8). It was determined that serum VDZ concentrations 23.2 mcg/ml at week 2 (the interquartile value between quartiles 2 and 3) was associated with endoscopic remission at week 52 (OR: 8.8 [95% CI: 2.6-29.7], p<0.001—FIG. 6) and clinical remission at week 14 (OR: 3.1 [95% CI: 1.01-9.3], p=0.044), week 22 (OR: 6.3 [95% CI: 1.9-20.7], p=0.002) and week 30 (OR: 5.0 [95% CI: 1.6-15.8], p<0.01). The interquartile serum VDZ concentration level values at week 6 were 12.5, 19.8 and mcg/ml (FIG. 9). The serum VDZ concentration ≥19.8 mcg/ml at week 6 (the interquartile value between quartiles 2 and 3) was also associated with endoscopic remission at week 52, even though the association was not as distinct when compared to serum VDZ concentrations at week 2 (OR: 3.1 [95% CI: 1.02-9.3], p=0.04). A serum VDZ concentration ≥19.8 mcg/ml at week 6 was also associated with clinical remission at week 14 (OR: 6.0 [95% CI: 1.9-19.4], p=0.002), week 22 (OR: 4.4 [95% CI: 1.4-13.8], p=0.01), and week 30 (OR: 7.1 [95% CI: 2.2-23.5], p<0.001). However, the survival analysis did not show a difference between groups (FIG. 7).

### Cross-Sectional Correlation Between VDZ Levels, Biomarkers, and Clinical Outcomes During Maintenance Therapy

There was no significant difference in median serum VDZ concentrations between patients in clinical remission and those with active disease at weeks 14 (11.2 [IQR:5.8-16.8] vs. 8.5 [IQR:6.2-12.1] mcg/ml, p=0.32), week 22 (8.8 [IQR:3.9-11.7] vs. 5.3 [IQR:3.5-12] mcg/ml, p=0.7), and week 30 (8.4 [IQR:5.1-10.5] vs. 5.2 [IQR:3.3-15.3] mcg/ml, p=0.8). A statistically significant negative correlation was found between serum VDZ concentrations and CRP at week 14 (rho: −0.34, p=0.039) and week 22 (rho: −0.37, p=0.03), but not at week 30 (rho: −0.3, p=0.21). Furthermore, a correlation between serum VDZ concentrations and serum albumin was identified at week 14 (rho: 0.55, p<0.001), but not at week 22 (rho: 0.23, p=0.25) or week 30 (rho: 0.5, p=0.09).

### VDZ Pharmacokinetics in Dose-Escalated Patients

Ten patients were dose-escalated from 300 mg every 8 weeks to 300 mg every 4 weeks. Of those, 6 had trough levels measured before and after dose escalation (all of them had UC). The median serum VDZ concentrations before and after dose escalation were 6.9 mcg/ml (IQR: 3.8-20.2) and 17.2 mcg/ml (IQR: 12.2-34.6), respectively. The median increase in serum VDZ concentrations was 9.1 mcg/ml (IQR: 7-15.7). Two of those patients (33%) did achieve endoscopic remission by week 52.

### Analysis of Results

These results may help to identify the group of patients who are more likely to have a good long-term response to VDZ therapy early in the course of treatment (during induction). These findings indicate that optimizing VDZ levels during induction may positively impact disease remission by one year. Although there was a trend towards higher trough serum VDZ concentrations during maintenance therapy in those patients that achieved endoscopic remission at week 52, it did not reach statistical significance. One potential explanation for this is the lack of statistical power to assess differences during maintenance. Other studies looking into clinical and endoscopic outcomes have observed similar results. Yacoub et al found that serum VDZ concentrations at week 6 were significantly higher in responders after one year (Yacoub et al. Aliment. Pharmacol. Ther. 2018, 369, 699-7). Differences between studies may be attributed to the use of standardized endoscopic scores for assessing mucosal healing and/or the type of assay used. Ungar et al reported an association between serum VDZ concentrations and clinical disease activity at week 6 (Ungar et al. Clinical Gastroenterology and Hepatology, 2018, 16, 697-705). Analysis from the GEMINI I and II pivotal studies also showed that response and remission to therapy was achieved at a faster rate in patients with higher serum VDZ concentrations. See, Feagan B. G., et al. N. Engl J Med 2013, 369, 699-710; Sandborn W. J., et al. N. Engl J Med 2013, 369, 711-721.

The heterogeneity in VDZ levels achieved by patient study groups and the wide spectrum of serum VDZ concentration changes in patients following dose escalation (4.8 to 16.1 mcg/ml) suggest significant heterogeneity in pharmacokinetic profiles. While a higher drug exposure may improve the response to VDZ therapy, another possibility is that those patients with a higher disease burden may have a higher drug clearance and exhibit lower drug levels. For example, those patients with lower rate of response may have higher clearance of drug due to a higher burden of systemic inflammation and/or a higher loss of drug through the gastrointestinal tract, as is seen with other biologics. See, Brandse, J. F., et al. Gastroenterology 2015, 149, 350-355.

Only three patients developed detectable ATV and were transient in all of them. Immunogenicity has been an important factor explaining non-response in patients receiving anti-TNF. See, Afif, W., et al. Am. J Gastroenterol. 2010, 105, 1133-1139. This low incidence maybe be due to the fact all patients in this study were starting the drug, and those that lost follow up or interrupted the treatment were excluded. The homogeneous-mobility shift assay used in this study is drug tolerant and can detect ATV in the presence of the drug so that would does not represent a limitation explaining the low rate of immunogenicity.

It was determined that baseline albumin had a significant correlation with drug levels throughout the follow-up period, which may be attributed to the fact that albumin is metabolized similarly to monoclonal antibodies. See, Yarur, A. J., et al. Inflamm. Bowel Dis. 2015, 21, 1709-1718. Albumin has also been found to be correlated in previous studies looking into the pharmacokinetics of VDZ. See, Rosario, M., et al. Aliment Pharmacol. Ther. 2015, 42, 188-202; Ungar et al. Clinical Gastroenterology and Hepatology, 2018, 16, 697-705; Yacoub et al. Aliment. Pharmacol. Ther. 2018, 369, 699-7. Another finding that matched previous reports on the pharmacokinetics of VDZ is the lack of a strong association between baseline CRP and drug levels. Furthermore, fecal calprotectin at baseline (week 0) negatively correlated with serum VDZ concentrations throughout induction. This association could be explained by the loss of protein through the gastrointestinal tract, especially when there is significant inflammation. See, Brandse, J. F., et al. Gastroenterology 2015, 149, 350-355.

It was observed that the patients that lost response by week 52 after having reached clinical remission by week 22 showed lower VDZ concentrations during maintenance but not during induction. This may support the use of TDM and dose escalation to recapture response when patients develop a disease exacerbation after being in remission. In the GEMINI trials, more than 50% of patients who lost response to VDZ on the Q8 week achieved clinical response after an increase in dosing frequency to Q4 weeks. See, Sands, B. E., et al. United European Gastroenterology Journal 2014, 2, Supplement 1.

While phenotypic characteristics are usually a spectrum, a patient with ileal CD may theoretically have a different pharmacokinetic profile when compared to a UC patient with pan-colitis. Sub-group analysis was not performed, but it was determined that patients with CD achieved higher drug levels throughout the study period.

In conclusion, an association between serum VDZ concentrations at induction and endoscopic remission at week 52 was found in this study. This correlation was stronger at week 2. Furthermore, a very low incidence of immunogenicity against the drug was observed, with 3 patients developing detectable anti-drug antibodies (all of which became undetectable by week 14). As with anti-TNF drugs, an overall positive correlation between serum VDZ concentrations and albumin was observed.

### Example 4: Association of Higher Trough Vedolizumab Concentrations During Maintenance Therapy with Corticosteroid-Free Remission in IBD

The following example demonstrates the association of maintenance VDZ levels with remission in IBD patients. More specifically, the aim of this study was to assess the association between trough serum VDZ concentrations and antibodies to VDZ (ATV) during maintenance therapy with clinical, biochemical and endoscopic disease activity in a multicenter cohort using a commercially available drug-tolerant assay.

### Methods

### Study Population and Design

A prospective cross-sectional study was performed in pediatric and adult patients with CD or UC receiving maintenance therapy with VDZ at the Mount Sinai Feinstein IBD Clinical Center (New York, N.Y.) or the Medical College of Wisconsin/Froedtert Hospital (Milwaukee, Wis.). The study was approved by the Institutional Review Boards at each site and all patients (or their parent) signed informed consent. Inclusion criteria were age 6 years or older, a confirmed diagnosis of UC or CD, and indication for VDZ being active disease defined as clinical symptoms (per patient report and medical record review) and elevated CRP. Patients with an ostomy or ileal pouch anal anastomosis were excluded. All patients had received induction with vedolizumab (300 mg at weeks 0, 2 and 6) and were in the maintenance phase of treatment (week 14 or later). Patients were all receiving 300 mg every 8 or 4 weeks depending on the decision of the treating physicians. At the time of enrollment, a complete medical history and assessment of clinical disease activity was completed. Clinical characteristics, laboratories and endoscopic data were extracted from the electronic medical record. CRP was routinely drawn prior to infusions at the study sites. Medical and medication history reported by patients was verified through review of the electronic medical record. All serum samples were drawn immediately prior to administration of VDZ maintenance dose. Coded samples were then sent to Prometheus Laboratories, Inc. (San Diego, Calif.) for analysis. Primary data were analyzed independently from the study sponsor.

### Measurement of VDZ Concentrations and ATVs

Serum levels of VDZ and ATV were measured using a validated, homogenous mobility shift drug tolerant assay (HMSA, Anser® VDZ, Prometheus Laboratories Inc., San Diego, Calif.). In brief, VDZ levels were measured by calculating the shift in antigen bound VDZ complex on a size exclusion column by HPLC. ATV levels were quantified using a size exclusion chromatography based mobility shift assay, run on a HPLC system with fluorescent detection. The lower limits of quantitation for vedolizumab and ATV were 1.6 μg/mL and 1.6 U/mL, respectively. The upper limits of quantitation for vedolizumab and ATV were 40 μg/mL and 75 U/mL, respectively. The laboratory conducting the measurements was blinded to all patient clinical data.

### Outcomes and Variables

The primary outcome was steroid-free clinical and biochemical remission defined as a composite of clinical remission, a normalized CRP, and no steroid use in the prior 4 weeks. Clinical remission was defined as a Harvey Bradshaw Index (HBI) score of less than 5 for CD or a partial Mayo Score (pMS) of 1 or less for UC. See, Harvey R F, Bradshaw J M. Lancet 1980, 1, 51412; Lewis J. D. et al. Inflamm Bowel Dis 2008, 14, 1660-1666. Normal serum CRP levels were defined as per local assay (≤5.0 mg/L at Mount Sinai or ≤0.5 mg/L at Medical College of Wisconsin). Secondary outcomes included steroid-free clinical remission, steroid-free CRP remission, steroid free endoscopic remission, and deep remission. Endoscopic remission was defined as a Simple Endoscopic Score for Crohn's Disease <3 or absence of ulcerations in CD patients or an endoscopic Mayo score <2 in UC patients on endoscopy performed within 8 weeks of infusion. Deep remission was defined as steroid-free clinical remission with normal CRP and endoscopic remission.

Independent variables included age, gender, race, smoking status, prior biologic exposure, prior IBD surgery, concomitant immunomodulator (methotrexate, 6-mercaptopurine, azathioprine) use, albumin level, VDZ infusion number, and disease location. Disease location was classified according to the Montreal classification with CD categorized as ileal (L1), colonic (L2), or ileo-colonic (L3) and UC categorized as proctitis (E1), left-sided disease (E2), or pancolitis (E3). See, Silverberg M. S. et al. Can J Gastroenterol 2005, 19, Suppl A: 5-36.

### Statistical Analysis

Descriptive statistics were performed to describe baseline characteristics of the study population which were reported as proportions or means for categorical and continuous variables, respectively. Average VDZ concentrations were reported as median with interquartile range (IQR) as they were nonparametric. Comparisons between VDZ concentrations in patients in remission and not in remission were performed using the Wilcoxon rank-sum test. Univariable analyses looking at the association between VDZ concentrations and independent variables with the pre-defined remission outcomes were performed using logistic regression. Receiver operating characteristic (ROC) analysis with determination of area under the curve (AUC) was also performed to assess the association of remission with VDZ concentrations. Multivariable logistic regression was then performed to assess the association of VDZ concentrations with each definition of remission while adjusting for potential confounders. Independent variables that were associated with the pre-defined remission outcomes that were significant at the p≤0.1 level were incorporated into the multivariable models. All analyses were performed using Stata 14.1 software (StataCorp, College Station, Tex.). Two-sided p values <0.05 were considered statistically significant.

### Results

### Study Cohort Characteristics

A total of 258 patients on VDZ maintenance therapy were enrolled in the study, 142 (55%) with CD and 116 (45%) with UC. Patient characteristics are described in Table 8. 55% had CD, the vast majority was white, 66% had prior biologic exposure, and mean disease duration was 10.8 years. The mean number of prior VDZ infusions was 7.2, corresponding to roughly 38 weeks on drug. The median VDZ concentration for the study cohort was 10.7 ug/mL (IQR 6.7-17) with a range from 0 to 43.3 ug/mL. ATVs were detected in 4 patients (1.6%). 3 of these patients had UC and 1 had CD while none were on an immunomodulator. Despite the presence of ATVs, all had detectable VDZ concentrations. Median VDZ concentrations in patients on immunomodulators were not significantly higher than those on monotherapy (11.1 ug/mL, IQR 5.8-17.2 vs. 10.6 ug/mL, IQR 6.9-16.8, p=0.68). Patients receiving VDZ every 4 weeks had significantly higher median concentrations than those on every 8 week dosing (15.0 ug/mL, IQR 8.5-24.1 vs. 10.2 ug/mL, IQR 6.3-15.2, p=0.003).

TABLE 8
Summary of patient characteristics (n = 258)

N (%)

Diagnosis

| | | |
|---|---|---|
| Crohn's disease | 142 | (55) |
| Ulcerative colitis | 116 | (45) |

Gender

| | | |
|---|---|---|
| Male | 135 | (52) |
| Female | 123 | (48) |

Race

| | | |
|---|---|---|
| White | 234 | (92) |
| Black | 11 | (4) |
| Asian | 7 | (3) |
| Other | 2 | (1) |

Montreal Classification for CD

| | | |
|---|---|---|
| L1 | 79 | (59) |
| L2 | 38 | (28) |
| L3 | 18 | (13) |

Montreal Classification for UC

| | | |
|---|---|---|
| E1 | 10 | (9) |
| E2 | 37 | (32) |
| E3 | 69 | (59) |

Prior Biologic Exposure

| | | |
|---|---|---|
| Yes | 169 | (66) |
| No | 89 | (34) |

Prior IBD Surgery

| | | |
|---|---|---|
| Yes | 64 | (25) |
| No | 194 | (75) |

Current Smoker

| | | |
|---|---|---|
| Yes | 12 | (5) |
| No | 246 | (95) |

Combination Therapy

| | | |
|---|---|---|
| Yes | 78 | (30) |
| No | 180 | (70) |

Other demographics

| | | |
|---|---|---|
| Age [mean in years (SD)] | 36 | (17) |
| Disease duration [mean in years (SD)] | 10.8 | (9.6) |
| Infusion number [mean (SD)] | 7.2 | (3) |
| HBI [mean score (SD)] | 3.4 | (4.3) |
| Partial Mayo Score (pMS) [mean (SD)] | 1.6 | (2.1) |
| Albumin [mean in g/dL (SD)] | 3.8 | (0.5) |

### Clinical and Biochemical Remission

A total of 83 (34%) patients met the primary outcome of corticosteroid-free clinical and biochemical remission while 161 (66%) did not. Ten patients who did not have CRP at time of trough VDZ concentration were excluded. As shown in Table 9, median VDZ concentrations were significantly higher in IBD patients meeting the primary outcome compared to those not in remission (12.7 ug/mL vs. 10.1 ug/mL, p=0.002). This difference in VDZ concentrations was more pronounced and significant in UC compared to CD patients (Table 9).

TABLE 9
Median VDZ concentrations and primary outcome¹

VDZ Concentration
[median in ug/mL (IQR)²]

IBD (p = 0.002)

| | | |
|---|---|---|
| Remission (n = 82) | 12.7 | (8.4-19.4) |
| No remission (n = 162) | 10.1 | (5.9-15.5) |

UC (p = 0.007)

| | | |
|---|---|---|
| Remission (n = 36) | 15.0 | (11-21.8) |
| No remission (n = 75) | 10.7 | (7.4-17.4) |

CD (p = 0.056)

| | | |
|---|---|---|
| Remission (n = 46) | 10.9 | (7.5-17.0) |
| No remission (n = 87) | 8.6 | (5.3-14.9) |

¹Clinical and CRP Corticosteroid-Free Remission;
²IQR: interquartile range

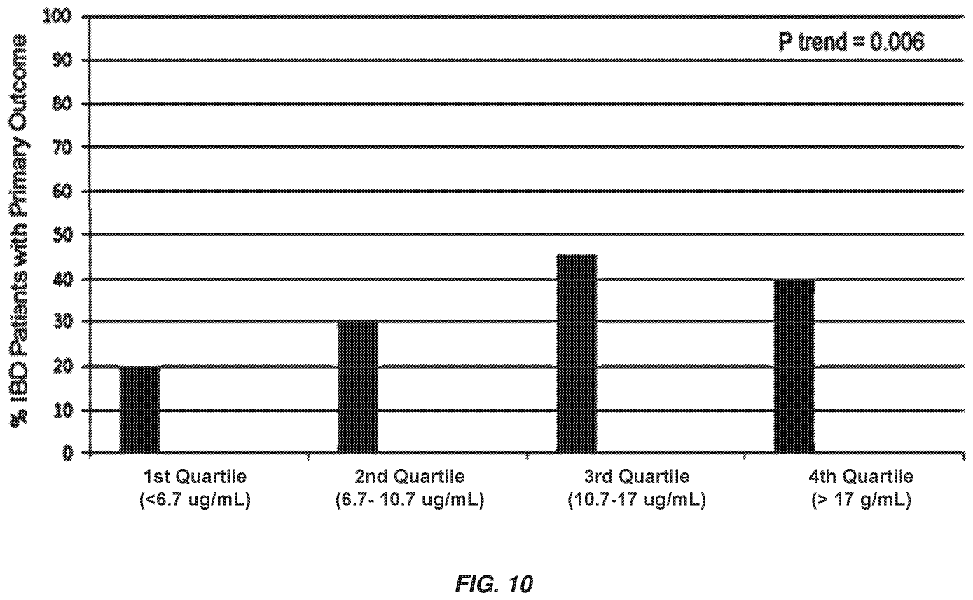

When examining the individual components of the primary outcome, the results appear to be primarily driven by corticosteroid free biochemical remission as VDZ concentrations did not significantly differ in IBD patients in corticosteroid free clinical remission (Table 10). VDZ concentrations were nearly identical when stratified by clinical remission in CD; however, trough VDZ concentrations were marginally significantly higher among UC patients in clinical remission (13.1 ug/mL vs. ug/mL, p=0.05). The primary outcome remission rates in IBD patients stratifying trough VDZ concentrations by quartile were then analyzed. Rates of remission were significantly higher with increasing VDZ quartile, with the 3rd and 4th quartiles having similar proportions of IBD patients with the composite outcome of clinical and biochemical remission (FIG. 10). Similar findings were seen with biochemical remission but not with clinical remission (data not shown).

TABLE 10
Median VDZ concentrations and corticosteroid-
free clinical remission

VDZ Concentration
[median in ug/mL (IQR)]

IBD (p = 0.15)

| | | |
|---|---|---|
| Remission (n = 124) | 11.4 | (7.3-16.8) |
| No remission (n = 134) | 10.2 | (6.3-17.2) |

UC (p = 0.05)

| | | |
|---|---|---|
| Remission (n = 51) | 15.0 | (11-21.8) |
| No remission (n = 65) | 10.7 | (7.4-17.4) |

CD (p = 0.60)

| | | |
|---|---|---|
| Remission (n = 73) | 9.9 | (6.0-16.4) |
| No remission (n = 69) | 9.6 | (5.7-15.7) |

### Endoscopic and Deep Remission

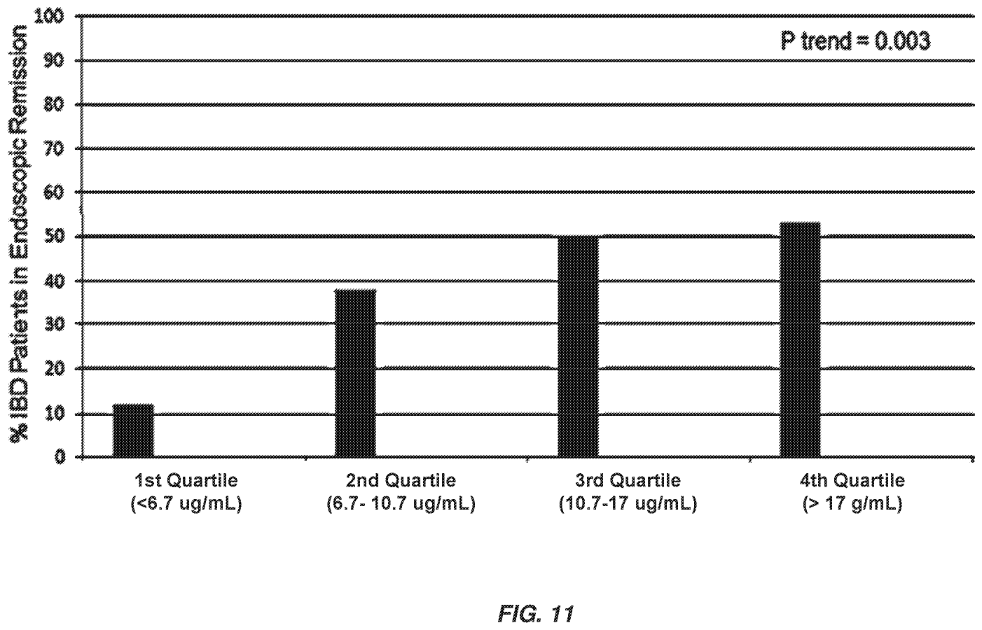

A subset of patients had sufficient data to evaluate corticosteroid-free endoscopic and deep remission. A total of 83 patients had endoscopic data (30 in remission and 53 with active disease) while deep remission was attained in 29 patients. Table 11 shows median VDZ concentrations were significantly higher for IBD patients in endoscopic remission (13.1 ug/mL vs. 8.5 ug/mL, p=0.01). While VDZ concentrations were numerically higher in UC patients in endoscopic remission this was not statistically significant. In contrast, CD patients in endoscopic remission had significantly higher concentrations than those not in remission (15.0 ug/mL vs. 7.5 ug/mL, p=0.008). In quartile analysis, higher VDZ concentration quartiles were associated with significantly higher rates of endoscopic remission (FIG. 11).

TABLE 11
Median VDZ concentrations and corticosteroid-
free endoscopic remission

VDZ Concentration
[median in ug/mL (IQR)]

IBD (p = 0.003)

| | | |
|---|---|---|
| Remission (n = 30) | 14.2 | (8.4-21.2) |
| No remission (n = 53) | 8.5 | (5.5-13.8) |

UC (p = 0.26)

| | | |
|---|---|---|
| Remission (n = 10) | 11.3 | (8.8-21.2) |
| No remission (n = 18) | 9.75 | (5.5-13.8) |

CD (p = 0.008)

| | | |
|---|---|---|
| Remission (n = 20) | 15.0 | (8.3-20.7) |
| No remission (n = 35) | 7.5 | (5.0-13.8) |

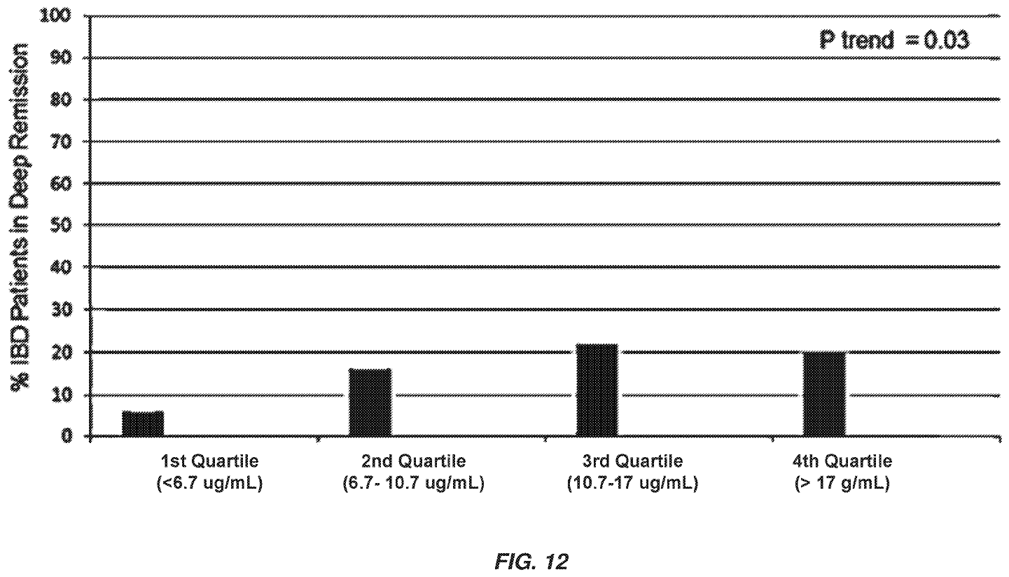

As shown in Table 12, a similar association with increased median VDZ concentrations was observed for those patients in corticosteroid-free deep remission (14.8 ug/mL vs. 9.4 ug/mL, p=0.005). UC patients in deep remission had a non-significant but numerically higher VDZ trough while VDZ was significantly higher in CD patients in deep remission compared to those with active disease (11.1 ug/mL vs. 10.7 ug/ml, p=0.4 and 15.0 ug/mL vs. 8.8 ug/mL, p=0.01). In quartile analysis, higher VDZ concentration quartiles were also associated with significantly higher rates of deep remission (FIG. 12).

TABLE 12
Median VDZ concentrations and corticosteroid-free deep remission

VDZ Concentration
[median in ug/mL (IQR)]

IBD (p = 0.01)

| | | |
|---|---|---|
| Remission (n = 29) | 14.8 | (8.4-21.2) |
| No remission (n = 160) | 10.1 | (5.9-15.1) |

UC (p = 0.40)

| | | |
|---|---|---|
| Remission (n = 9) | 11.1 | (8.8-21.2) |
| No remission (n = 70) | 10.7 | (7.2-15.2) |

CD (p = 0.01)

| | | |
|---|---|---|
| Remission (n = 20) | 15.0 | (8.3-20.5) |
| No remission (n = 90) | 8.8 | (5.3-15) |

### Multivariate Analysis of Remission

In order to understand if trough VDZ concentrations were independently associated with measures of remission, other clinical variables with potential impact on remission rates in univariable logistic regression were considered. Results for these variables for the primary outcome, endoscopic remission and deep remission are shown in Table 13.

TABLE 13
Univariable association of covariables for all IBD patients

| | |
|---|---|
| OR¹ (95% CI) | P value |

Primary Outcome

| | | | |
|---|---|---|---|
| Diagnosis (Reference = UC) | 1.10 | (0.65-1.88) | 0.72 |
| Age | 0.98 | (0.97-0.99) | 0.032 |
| Albumin | 0.99 | (0.88-1.11) | 0.87 |
| Disease duration | 0.99 | (0.97-1.02) | 0.67 |
| Hx IBD surgery | 1.05 | (0.57-1.96) | 0.87 |
| Smoker | 1.15 | (0.33-4.05) | 0.83 |
| Gender (Reference = Female) | 0.78 | (0.46-1.33) | 0.36 |
| Prior biologic (Reference = No) | 0.44 | (0.25-0.76) | 0.003 |
| Combination therapy | 0.90 | (0.50-1.62) | 0.72 |
| Escalated VDZ frequency | 0.40 | (0.18-0.87) | 0.021 |
| Number of prior infusions | 1.01 | (0.94-1.08) | 0.84 |

Corticosteroid-Free Endoscopic Remission

| | | | |
|---|---|---|---|
| Diagnosis (Reference = UC) | 1.03 | (0.40-2.66) | 0.95 |
| Age | 1.00 | (0.98-1.03) | 0.74 |
| Albumin | 1.76 | (0.57-5.43) | 0.32 |
| Disease duration | 0.97 | (0.92-1.02) | 0.25 |
| Hx IBD surgery | 0.66 | (0.22-1.60) | 0.31 |
| Smoker | 1.07 | (0.24-4.81) | 0.93 |
| Gender (Reference = Female) | 0.51 | (0.21-1.27) | 0.15 |
| Prior biologic (Reference = No) | 0.37 | (0.14-0.96) | 0.04 |
| Combination therapy | 2.22 | (0.87-5.64) | 0.09 |
| Escalated VDZ frequency | 0.60 | (0.15-2.45) | 0.46 |
| Number of prior infusions | 0.98 | (0.80-1.19) | 0.82 |

Deep Remission

| | | | |
|---|---|---|---|
| Diagnosis (Reference = UC) | 1.73 | (0.74-4.03) | 0.21 |
| Age | 1.01 | (0.99-1.03) | 0.41 |
| Albumin | 1.02 | (0.89-1.16) | 0.79 |
| Disease duration | 0.98 | (0.93-1.03) | 0.36 |
| Hx IBD surgery | 1.18 | (0.49-2.88) | 0.71 |
| Smoker | 2.52 | (0.61-10.38) | 0.20 |
| Gender (Reference = Female) | 0.56 | (0.25-1.26) | 0.16 |
| Prior biologic (Reference = No) | 0.48 | (0.21-1.08) | 0.08 |
| Combination therapy | 1.90 | (0.85-4.25) | 0.12 |
| Escalated VDZ frequency | 0.35 | (0.10-1.21) | 0.10 |
| Number of prior infusions | 0.81 | (0.67-0.98) | 0.03 |

¹OR = odds ratio

Each covariable having a p value less than or equal to 0.10 in the univariable analysis (Table 13) was then adjusted. All IBD patients were included and all endpoints were corticosteroid-free. Such variables significant at the preset p value were then analyzed with trough VDZ concentration in multivariable models for each outcome of interest. All IBD patients were included and all endpoints were corticosteroid-free. Trough VDZ concentration was dichotomized as above or below the median (10.7 ug/mL) since quartile analysis demonstrated similar remission rates for the 3rd and 4th quartiles and both upper quartile rates were higher than the bottom two quartiles. After controlling for possible confounding variables, trough VDZ concentration above the median was still significantly associated with an increased chance of achieving remission in each of the examined outcome definitions (Table 14).

TABLE 14
Multivariable association of VDZ concentration
above median¹ with remission²

| | |
|---|---|
| Adjusted OR³ (95% CI) | P value |

| | | | |
|---|---|---|---|
| Primary outcome | 2.45 | (1.38-4.37) | 0.002 |
| Endoscopic remission | 3.99 | (1.42-11.20) | 0.009 |
| Deep remission | 2.45 | (1.05-5.71) | 0.04 |

¹median = 10.7 ug/mL;
²Remission types based on univariable association are defined as the following: primary outcome = age, escalated VDZ dosing, prior biologic; endoscopic remission = combination therapy, prior biologic; deep remission = prior biologic, number prior infusions, escalated VDZ dosing;
³OR = odds ratio

### ROC Analysis

ROC analysis was performed to further characterize the performance of VDZ concentration with different definitions of remission. For the primary outcome, VDZ concentrations had an AUC of 0.62. For secondary outcomes, VDZ concentrations had an AUC of 0.70 for endoscopic remission and an AUC of 0.64 for deep remission.

### Analysis of Results

In a large multicenter cohort of IBD patients, trough VDZ concentrations measured using a drug tolerant assay were significantly associated with remission during maintenance VDZ treatment. Immunogenicity was very low with only 1.6% of patients having ATVs. Patients with VDZ concentrations above 10.7 ug/mL were 2 to 4 times more likely to be in clinical and biochemical, endoscopic, and deep remission after adjusting for potential confounders. Based on readily available resources in the field, this is the largest reported real-world cohort on VDZ concentrations to date.

The results offer data to help guide clinical decision making during maintenance therapy, particularly at the time of loss of response to VDZ when checking drug concentrations is advocated by national gastroenterology societies. See, Feuerstein J. D., et al. Gastroenterology 2017, 153, 827-834. If a patient is not responding to VDZ during maintenance, the results described above suggest that dose escalation should be considered in order to achieve a concentration of at least 11 ug/mL. The data also suggest that even higher concentrations may be necessary to achieve endoscopic and deep remission particularly in CD patients. Median VDZ concentrations among CD patients in endoscopic and deep remission were 15 ug/mL, while in UC the median was lower at 11 ug/mL, suggesting that CD patients may require higher trough VDZ concentrations during maintenance.

Using a drug tolerant assay, very low rates of immunogenicity to VDZ were observed. These results with a drug tolerant assay suggest that the rates of immunogenicity are very low with VDZ. The potential implication is that combination with immunomodulators may not be necessary to prevent immunogenicity with VDZ. The concomitant immunomodulator therapy did not appear to have any impact on VDZ concentrations. One possible explanation for this is that the primary impact of immunomodulator therapy in combination with biologics may be to limit immunogenicity to the biologic, rather than promote a synergistic effect through an alternative mechanism of action. See, Colombel, J. F., et al. Gastroenterology 2017, 152(5), Supplement 1: S37-S38. The results from this study also suggest that increased VDZ concentrations well above 3 ug/mL is associated with improved response and remission rates. Therefore, the mechanism of VDZ may not be solely depending on complete blockade of peripheral a4β7 expressing T cells.

In conclusion, the results of this study showed that maintenance VDZ concentrations are significantly associated with remission in IBD patients in a large real-world cohort. VDZ appears to have low immunogenicity. When controlling for potential confounders, trough VDZ concentrations of 11 ug/mL or higher, as determined with a drug tolerant HMSA assay, were significantly associated with remission defined by objective measures including CRP and endoscopy.

### Example 5: Association of Clinical and Endoscopic Outcomes with Vedolizumab Treatment in Ulcerative Colitis

The following example demonstrates the association of serum biomarkers (i.e., s-TNF, s-α4β7, s-MAdCAM-1, CRP, s-AA, s-ICAM-1, s-VCAM-1) with outcomes in vedolizumab-treated UC patients. The serum biomarkers, vedolizumab (VDZ) concentrations, and anti-VDZ antibodies (ATV) were analyzed throughout VDZ therapy in UC patients. The VDZ and biomarker concentrations were compared to maintenance clinical and endoscopic outcomes.

### Methods

### Patient Selection, Ethics, and Sample Collection

Eligible subjects were adults with a confirmed UC diagnosis, undergoing VDZ treatment at the University of California, San Diego IBD Center. Patients received VDZ 300 mg intravenously during induction at week 0, 2, 6, then maintenance infusions every 8 weeks. Dose-escalation to every 4 weeks was based on provider assessment after induction. The protocol was approved by the local institutional review board and patients provided informed consent prior to enrollment. Serum was prospectively obtained immediately prior to VDZ infusions during induction at week 0 (baseline), 2, 6 and during maintenance at week 14 and ≥26 between January 2014 and October 2016. Biomarker analysis included one patient sample per time point.

### Endpoints and Definitions

Clinical and endoscopic scoring was performed prospectively. Disease extent was defined using the Montreal classification. See, Silverberg M. S. et al. Can J Gastroenterol 2005, 19, Suppl A: 5-36. Outcomes included: clinical remission (physician global assessment (PGA)=0 and neither treatment discontinuation nor colectomy) and endoscopic remission (Mayo endoscopic sub-scores (ESS) of 0 or 1). Biomarkers concentrations at baseline (week 0), weeks 2, 6, 14 and ≥26 were compared between remitters and non-remitters for clinical and endoscopic outcomes assessed during maintenance. Correlations between vedolizumab and individual biomarker concentrations, as well as between s-TNF and s-ICAM-1, s-VCAM-1 and s-α4β7 were explored. This was performed separately during induction and maintenance using the latest available sample collection in each phase. Corticosteroid effects on biomarker concentrations were evaluated. Baseline biomarker concentrations were compared based on baseline systemic corticosteroid requirement. This was repeated for corticosteroid requirement at ≥week 26.

### Biomarker Assays

Vedolizumab and ATV measurements used a homogenous mobility shift assay (HMSA), Anser® VDZ (Prometheus Laboratories Inc., San Diego, Calif.). Serum TNF measurements used the Erenna® SMC™ Human TNFα Immunoassay Kit (EMD Millipore, St. Charles, Mo.). CRP, s-AA, s-ICAM-1 and s-VCAM-1 measurements used V-Plex Vascular Injury Panel-2 (human) Kits (Meso Scale Discovery, Rockville, Md.). sMAdCAM-1 and s-α4β7 measurements used enzyme-linked immunosorbent assays (ELISA, Prometheus Laboratories Inc.).

### Statistical Analysis

Continuous variables were summarized by mean±standard deviation and median with interquartile rage (IQR), respectively, for normally- and non-normally-distributed data. Percentages were used for categorical variables. Between-groups comparisons were performed using Fishers exact test and the Mann-Whitney U test for categorical and continuous independent data, with the Wilcoxon signed-rank test used for paired continuous data. Linear mixed-effects models were fit with each biomarker as the outcome and included baseline biomarker values, time (continuous), and remission status as covariates, with an interaction term between time and remission status to investigate differences in longitudinal trends. Likelihood ratio tests were used to choose a parsimonious random effects structure for each model, and an independent random intercept and slope was indicated in each case. P-values ≤0.05 were considered significant. Spearman correlation coefficients were calculated between VDZ and individual biomarker concentrations, as well as between s-TNF and s-ICAM-1, s-VCAM-1 and s-α4β7. Analyses were performed using GraphPad Prism version 7.03 (GraphPad Software, California, USA) or R (lme4 package, for linear mixed-effects models). See, R: A Language and Environment for Statistical Computing [computer program]. Vienna, Austria: R Foundation for Statistical Computing, 2017; Bates, D., et al. Journal of Statistical Software, Articles. 2015, 67, 1-48.

### Results

### Patients and Patient Outcomes

Of thirty-two included patients (baseline samples: n=18, week 2: n=12, week 6: n=14, week 14: n=16, ≥week 26: n=20), 81% had extensive colitis, 56% had severe endoscopic baseline disease (EES=3) and 84.4% had prior TNF-antagonist exposure (Table 15). At baseline, 34% received concomitant immunosuppression (i.e., azathioprine, mercaptopurine, methotrexate, or mycophenolate-mofetil). Median time to assessment for clinical remission was 26.5 weeks (IQR: 16.3-37.0), and 23.5 weeks (IQR: 16.8-35.6) for endoscopy.

TABLE 15
Patient demographics

| Baseline Demographics | N = 32 | |
|---|---|---|
| Age [mean in years (SD)] | 46.4 | (18.2) |
| Female gender [n (%)] | 8 | (31.3) |
| Diagnosed at age <16 years [n (%)] | 2 | (6.3) |
| Diagnosed at age 16-40 years [n (%)] | 16 | (50.0) |
| Diagnosed at age >40 years [n (%)] | 14 | (43.8) |
| Disease duration [mean in years (SD)] | 7.7 | (7.1) |
| Disease extent - Proctitis [n (%)] | 2 | (6.3) |
| Disease extent - Left-sided colitis [n (%)] | 4 | (12.5) |
| Disease extent - Extensive colitis [n (%)] | 26 | (81.3) |
| Current smoker [n (%)] | 2 | (6.3) |
| Body mass index [mean (SD)] | 26.8 | (6) |
| Prior TNF inhibitor use [n (%)] | 27 | (84.4) |
| Baseline albumin [mean in g/dL (SD)] | 4 | (0.3) |
| Baseline partial Mayo score [mean (SD)] | 6 | (1.9) |
| Baseline Mayo score endoscopic sub-score [mean (SD)] | 2.5 | (0.7) |
| Baseline corticosteroid requirement [n (%)] | 22 | (68.8) |
| Concomitant immunomodulator use [n (%)] | 11 | (34.4) |
| Dose escalation [n (%)] | 16 | (50.0) |

At time of assessment, fourteen patients (46.7%) achieved clinical remission, 19 (59.4%) achieved endoscopic remission, 20 (74.1%) were corticosteroid-free and 16 (50.0%) underwent dose-escalation (median time: 26 weeks, IQR: 19-56). The proportion of TNF antagonist naïve patients was not significantly different between patients achieving endoscopic remission (10.5%, n=19) and those not achieving endoscopic remission (21.4%, n=14, p=0.40).

### VDZ and Anti-VDZ (ATV) Concentrations

Median VDZ concentrations at weeks 2, 6, 14 and 26 were 20 mcg/mL (IQR:16-27), 20 mcg/mL (IQR: 9-25), 12 mcg/mL (IQR: 7-17) and 10 mcg/mL (IQR: 8-27), respectively. ATV were detected in 2 patients (5.9%). Both received VDZ monotherapy, with ATV detection at week 2. In one patient, antibodies persisted, dose-escalation failed and colectomy was required. Another patient transiently developed antibodies until week 6, with detectable vedolizumab. Dose-escalation resulted in MH without further ATV. Although vedolizumab concentrations at any time point were not significantly associated with week 26 outcomes (Table 16), induction concentrations were numerically higher in clinical and endoscopic remitters. ATV's were not associated with outcomes.

TABLE 16
Associations of VDZ concentrations at weeks 2, 6, 14 and 26 with week 26 outcomes.

| | Median | IQR 25-75 | n | Median | IQR 25-75 | n | p value |
|---|---|---|---|---|---|---|---|
| | Clinical Remission | | | No Clinical Remission | | | |
| VDZ week 2 [ug/ml] | 19.4 | 17.0, 24.2 | 5 | 17.5 | 13.6, 32.6 | 6 | 0.58 |
| VDZ week 6 [ug/ml] | 22 | 19.3, 26.1 | 7 | 9.1 | 7.3, 14.6 | 5 | 0.22 |
| VDZ week 14 [ug/ml] | 11.7 | 6.8, 17.0 | 7 | 9.9 | 3.0, 13.3 | 6 | 1.00 |
| VDZ week 26 [ug/ml] | 10.2 | 8.4, 31.9 | 10 | 10.3 | 7.8, 22.2 | 10 | 0.97 |
| | Endoscopic Remission | | | No Endoscopic Remission | | | |
| VDZ week 2 [ug/ml] | 21.7 | 16.9, 27.3 | 8 | 17.5 | 13.8, 35.9 | 4 | 0.61 |
| VDZ week 6 [ug/ml] | 22 | 19.0, 26.7 | 9 | 11.9 | 8.7, 17.3 | 4 | 0.36 |
| VDZ week 14 [ug/ml] | 13.3 | 8.2, 18.1 | 10 | 8 | 1.3, 11.7 | 5 | 0.24 |
| VDZ week 26 [ug/ml] | 10.6 | 8.3, 32.2 | 13 | 9 | 8.1, 19.1 | 7 | 0.90 |

### Biomarkers

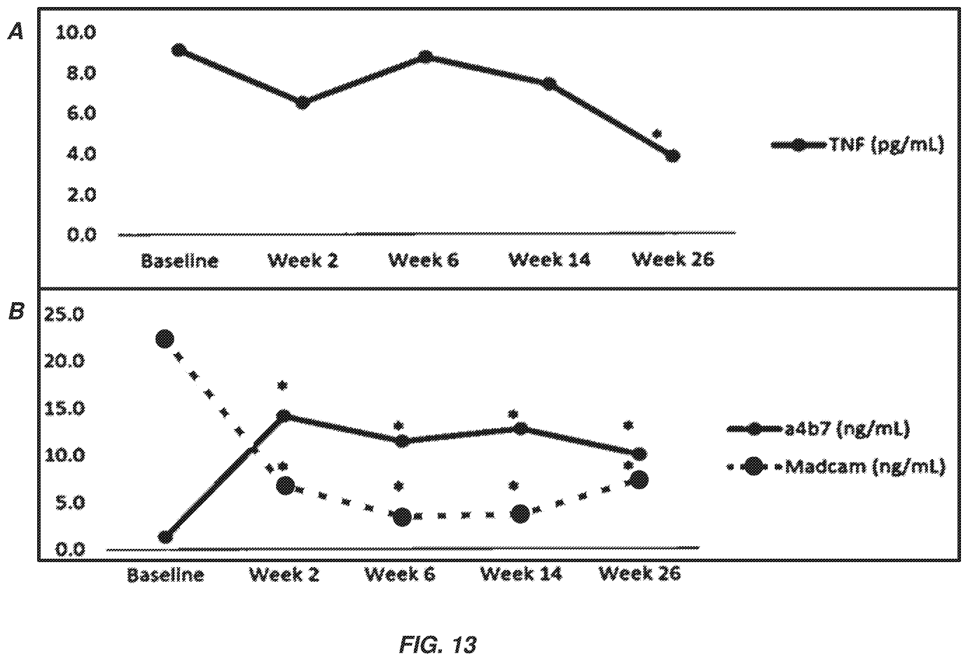

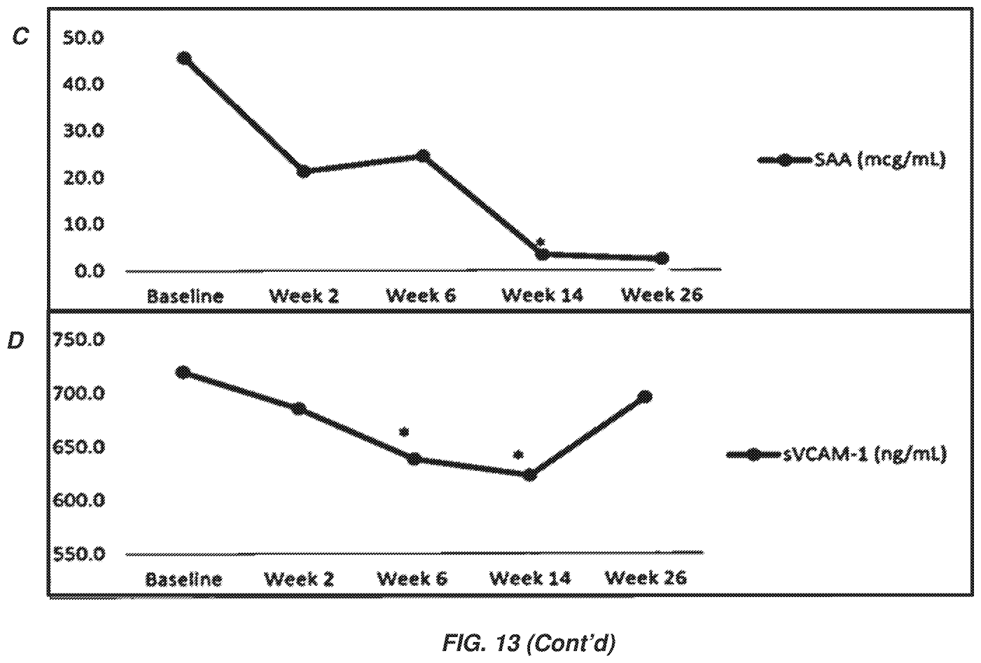

For each biomarker, three analyses were performed. First, changes in biomarker concentrations with treatment are reported for all patients (FIG. 13, Table 17). In patients with baseline biomarkers prior to VDZ therapy, s-TNF concentrations decreased at week 26 (FIG. 13-A). The concentrations of s-α4β7 and s-MAdCAM-1 significantly changed at every time point measured (FIG. 13-B). As shown in FIG. 13-C, s-AA concentrations significantly decreased at week 14 and trended towards lower concentrations at week 26, with lower samples sizes than previous time points (Table 17). The s-VCAM-1 concentrations changed at week 6 and 14, but these changes did not persist later during maintenance at week 26 (FIG. 13-D).

TABLE 17
Changes in biomarkers with VDZ therapy¹

| | |
|---|---|
| n | p value |

Baseline biomarkers

| | | | |
|---|---|---|---|
| TNF (pg/mL) | 9.1 | 17 | na |
| α4β7 (ng/mL) | 1.3 | 18 | na |
| Madcam (ng/mL) | 22.3 | 18 | na |
| SAA (mcg/mL) | 45.5 | 18 | na |
| sVCAM-1 (ng/mL) | 719.3 | 18 | na |

Week 2 biomarkers

| | | | |
|---|---|---|---|
| TNF (pg/mL) | 6.5 | 10 | NS |
| α4β7 (ng/mL) | 14.1 | 11 | <0.001 |
| Madcam (ng/mL) | 6.7 | 11 | <0.001 |
| SAA (mcg/mL) | 21.2 | 11 | NS |
| sVCAM-1 (ng/mL) | 684.9 | 11 | NS |

Week 6 biomarkers

| | | | |
|---|---|---|---|
| TNF (pg/mL) | 8.7 | 13 | NS |
| α4β7 (ng/mL) | 11.4 | 13 | <0.001 |
| Madcam (ng/mL) | 3.4 | 13 | <0.001 |
| SAA (mcg/mL) | 24.4 | 13 | NS |
| sVCAM-1 (ng/mL) | 637.8 | 13 | 0.002 |

Week 14 biomarkers

| | | | |
|---|---|---|---|
| TNF (pg/mL) | 7.4 | 12 | NS |
| α4β7 (ng/mL) | 12.7 | 13 | <0.001 |
| Madcam (ng/mL) | 3.6 | 13 | <0.001 |
| SAA (mcg/mL) | 3.3 | 13 | 0.025 |
| sVCAM-1 (ng/mL) | 622.5 | 13 | 0.003 |

Week 26 biomarkers

| | | | |
|---|---|---|---|
| TNF (pg/mL) | 3.8 | 8 | 0.038 |
| α4β7 (ng/mL) | 9.9 | 8 | 0.002 |
| Madcam (ng/mL) | 7.3 | 7 | 0.004 |
| SAA (mcg/mL) | 2.3 | 8 | 0.095 |
| sVCAM-1 (ng/mL) | 695.0 | 8 | NS |

¹In patients with baseline biomarkers prior to VDZ therapy, the concentrations and number of patients with sample at each time point is provided. This data corresponds to FIG. 13.

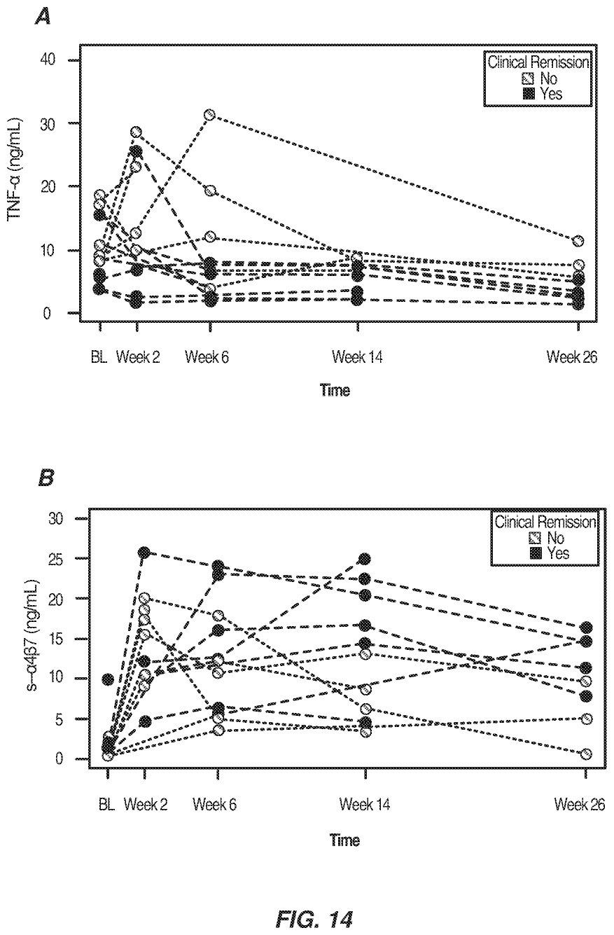

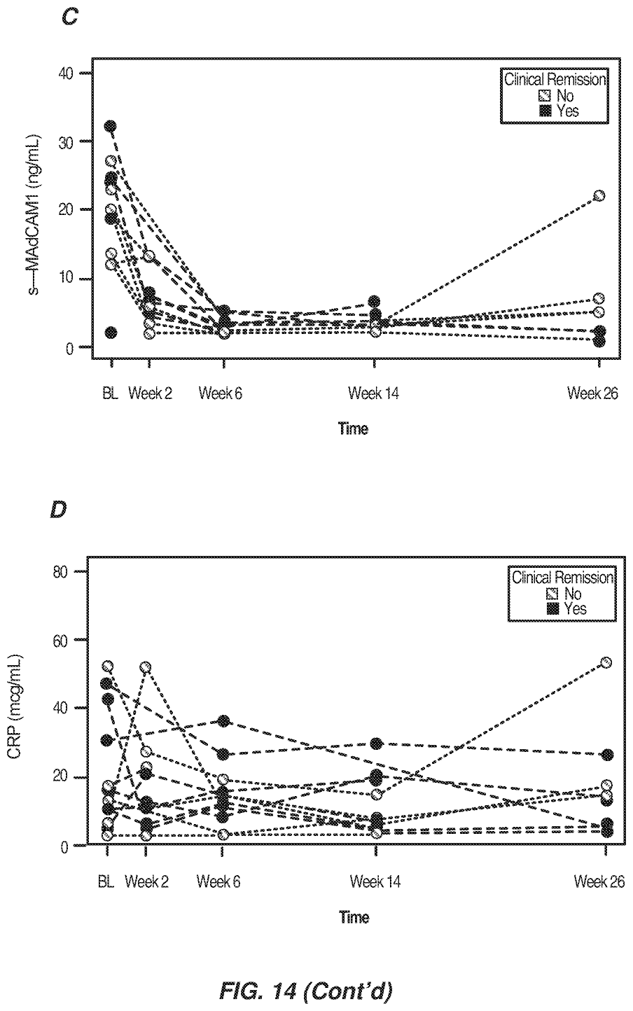

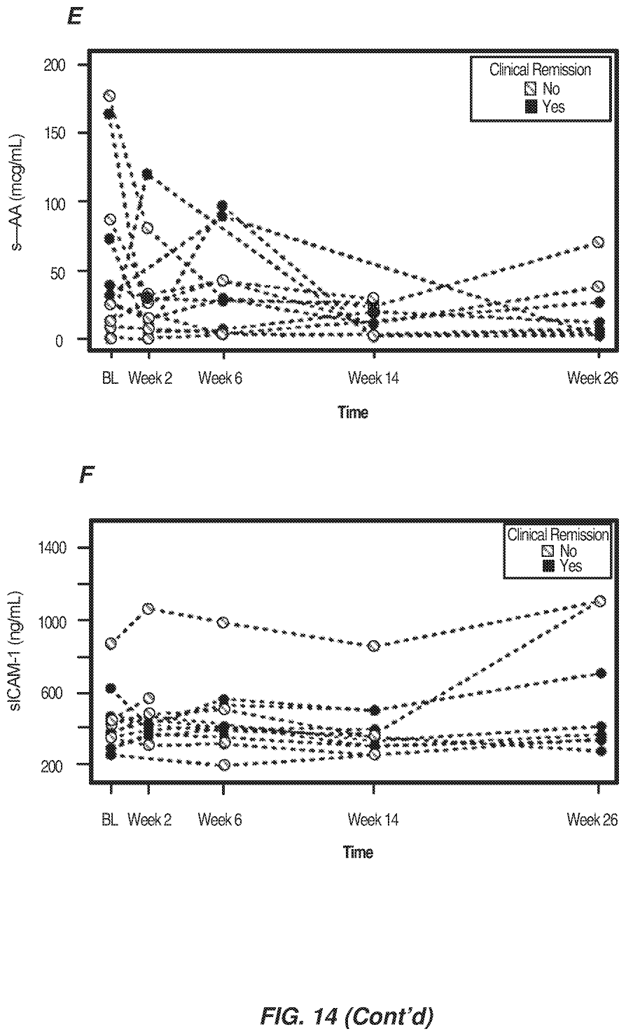

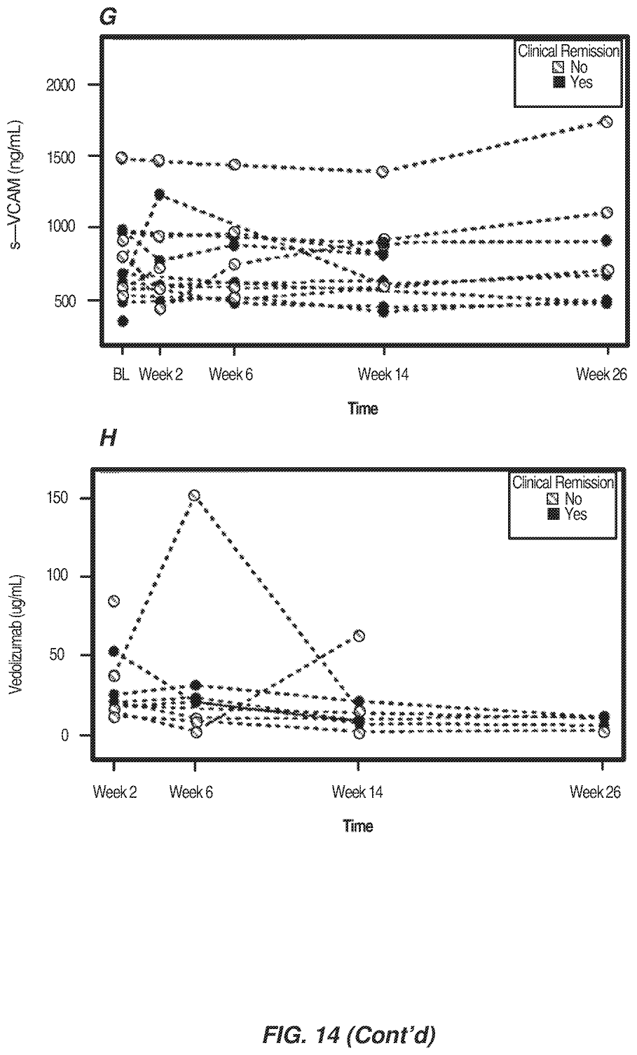

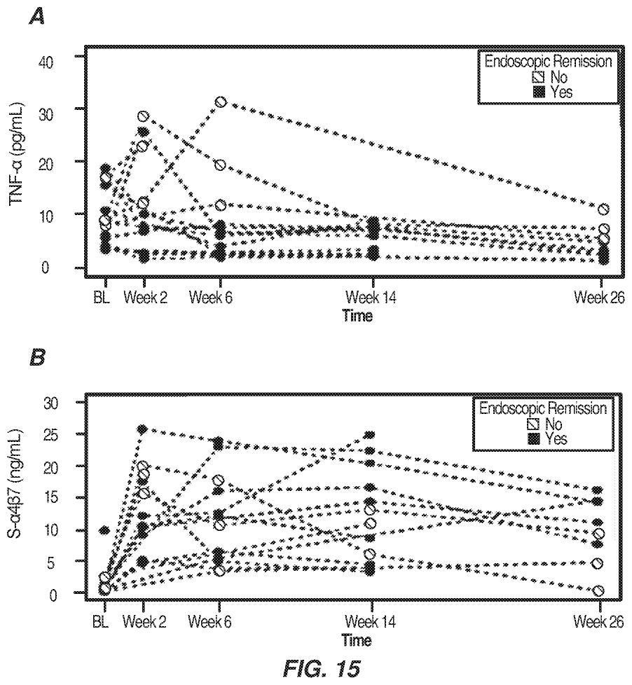

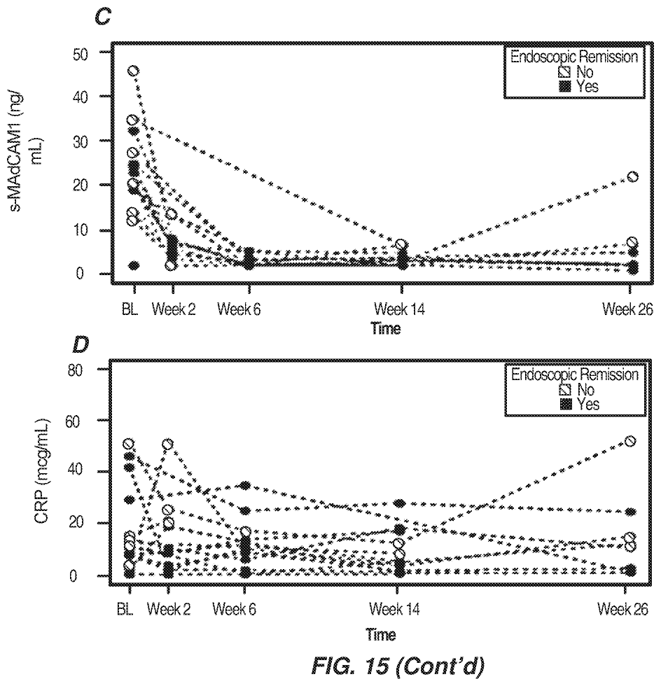

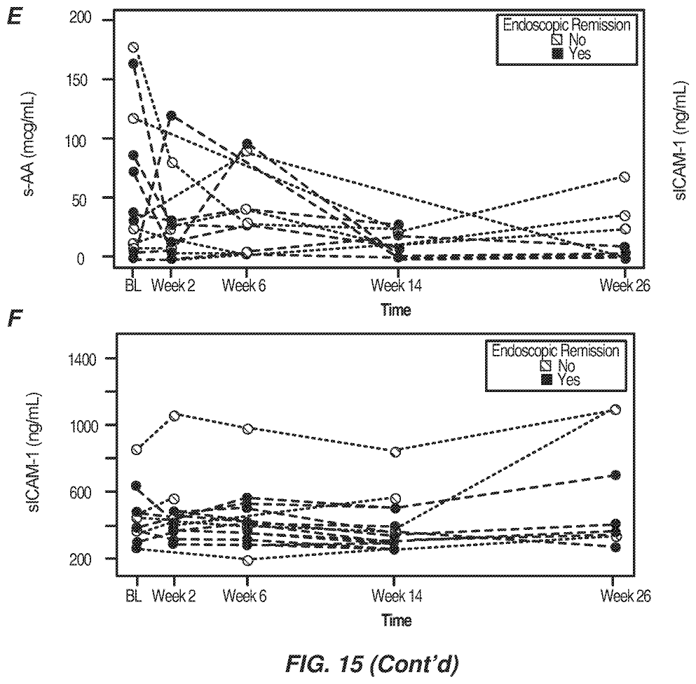

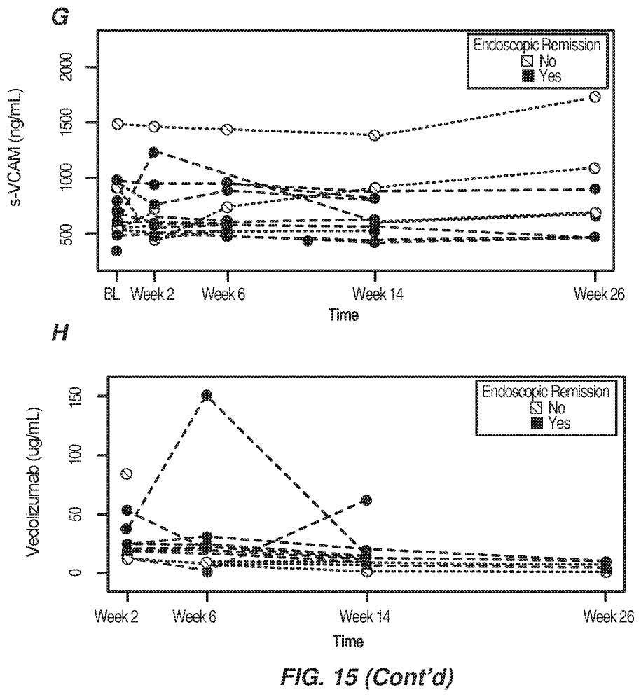

Second, comparisons for biomarker trajectories over time between clinical or endoscopic remitters to non-remitters are described (using linear mixed-effects models, FIG. 14 and FIG. 15). For all mixed models fit, a random intercept alone was determined to be the best random-effect structure. The rate (slope) of increase or decrease between groups was compared (herein referred as more rapid increase or decline). As shown in FIG. 14, a significant interaction between time and clinical remission was observed with s-α4β7 (p=0.044, FIG. 14-B), s-MAdCAM-1 (p=0.006, FIG. 14-C), and s-VCAM-1 (p=0.001, FIG. 14-G), with s-α4β7 increasing faster in those who achieved clinical remission, and s-MAdCAM-1 and s-VCAM-1 decreasing faster in those who achieved clinical remission. This trend was not observed with s-TNF (p=0.22, FIG. 14-A), CRP (p=0.44, FIG. 14-D), s-AA (p=0.07, FIG. 14-E), s-ICAM-1 (p=0.07, FIG. 14-F), or vedolizumab concentrations (p=0.49, FIG. 14-H). A significant interaction between time and endoscopic remission was observed for s-MAdCAM-1 (p=0.005, FIG. 15-C), s-ICAM-1 (p=0.014, FIG. 15-F), and s-VCAM-1 (p<0.001, FIG. 15-G), with each decreasing faster in those who achieved endoscopic remission. A similar trend was found with s-TNF concentrations (p=0.052, FIG. 15-A) and s-α4β7 (p=0.07, FIG. 15-B), but not for CRP (p=0.075, FIG. 15-D), s-AA (p=0.14, FIG. 15-E), or vedolizumab (p=0.47, FIG. 15-H) concentrations.

Lastly, comparisons of biomarker concentrations measured at weeks 2, 6, 14 and 26 between clinical or endoscopic remitters to non-remitters were reported (Table 18, Table 19, Table 20, and Table 21, respectively). Biomarker concentrations measured at weeks 2, 6, 14, and ≥week 26 were stratified based on the achievement of clinical and endoscopic remission during maintenance. Individual median baseline biomarker concentrations (s-TNF, s-α4β7, s-MAdCAM-1, CRP, s-AA, s-ICAM-1, s-VCAM-1) were not significantly different between groups for any outcomes.

TABLE 18
Associations of week 2 biomarkers with maintenance outcomes.

| Biomarker | Median | IQR 25-75 | n | Median | IQR 25-75 | n | p value |
|---|---|---|---|---|---|---|---|
| | Clinical Remission | | | No Clinical Remission | | | |
| TNF-α (pg/mL) | 6.7 | 2.4, 7.5 | 5 | 12.5 | 10.6, 23.2 | 5 | NS |
| α4β7 (ng/mL) | 10.2 | 9.1, 12.1 | 5 | 16.5 | 15.6, 18.3 | 6 | NS |
| α4β7 (change¹, ng/mL) | 9.7 | 7.2, 11.9 | 5 | 16.1 | 12.9, 18.1 | 5 | NS |
| MAdCAM1 (ng/mL) | 7.1 | 6.6, 7.9 | 5 | 5.2 | 3.8, 6.2 | 6 | NS |
| SAA (mcg/mL) | 14.7 | 5.4, 29.7 | 5 | 29.3 | 12.8, 41.8 | 6 | NS |
| sVCAM-1 (ng/mL) | 599.4 | 573.5, 771.5 | 5 | 658.0 | 585.9, 884.0 | 6 | NS |
| s-VCAM (change¹, ng/mL) | −2.5 | −22.6, 78.0 | 5 | −36.4 | −213.9, −20.4 | 5 | NS |
| CRP (mcg/mL) | 8.3 | 3.6, 10.2 | 5 | 15.3 | 8.9, 24.3 | 6 | NS |
| sICAM-1 (ng/mL) | 396.1 | 375.7, 421.6 | 5 | 465.5 | 435.0, 547.5 | 6 | NS |
| | Endoscopic Remission | | | No Endoscopic Remission | | | |
| TNF-α (pg/mL) | 6.7 | 2.5, 8.8 | 7 | 17.8 | 12.1, 24.5 | 4 | 0.038 |
| α4β7 (ng/mL) | 10.4 | 8.1, 13.4 | 8 | 17.1 | 15.6, 18.9 | 4 | NS |
| α4β7 (change¹, ng/mL) | 10.0 | 6.6, 13.0 | 8 | 18.1 | 15.5, 18.9 | 3 | NS |
| MAdCAM1 (ng/mL) | 6.8 | 5.6, 7.7 | 8 | 5.4 | 3.9, 8.0 | 4 | NS |
| SAA (mcg/mL) | 11.1 | 5.2, 30.4 | 8 | 35.7 | 21.9, 53.8 | 4 | NS |
| sVCAM-1 (ng/mL) | 591.4 | 571.8, 813.0 | 8 | 658.0 | 552.9, 909.1 | 4 | NS |
| s-VCAM (change¹, ng/mL) | −29.5 | −168.5, 17.6 | 8 | −20.4 | −247.6, 65.2 | 3 | NS |
| CRP (mcg/mL) | 6.0 | 2.0, 9.0 | 8 | 23.1 | 18.1, 31.9 | 4 | 0.017 |
| sICAM-1 (ng/mL) | 385.9 | 347.8, 434.4 | 8 | 508.6 | 443.8, 693.9 | 4 | 0.042 |

¹change from baseline

TABLE 19
Associations of week 6 biomarkers with maintenance outcomes.

| Biomarker | Median | IQR 25-75 | n | Median | IQR 25-75 | n | p value |
|---|---|---|---|---|---|---|---|
| | Clinical Remission | | | No Clinical Remission | | | |
| TNF-α (pg/mL) | 5.9 | 2.5, 7.1 | 7 | 11.9 | 6.6, 19.3 | 5 | NS |
| α4β7 (ng/mL) | 12.5 | 6.1, 19.5 | 7 | 10.6 | 4.9, 12.1 | 5 | NS |
| α4β7 (change¹, ng/mL) | 13.2 | 7.7, 20.0 | 6 | 7.9 | 3.6, 11.9 | 5 | NS |
| MAdCAM1 (ng/mL) | 2.0 | 2.0, 3.4 | 7 | 2.0 | 2.0, 2.7 | 5 | NS |
| SAA (mcg/mL) | 27.5 | 5.5, 59.0 | 7 | 29.6 | 3.9, 42.0 | 5 | NS |
| sVCAM-1 (ng/mL) | 616.0 | 565.3, 769.9 | 7 | 742.4 | 580.4, 962.3 | 5 | NS |
| s-VCAM (change¹, ng/mL) | −59.1 | −94.6, −16.4 | 6 | −49.5 | −165.6, −35.4 | 5 | NS |
| CRP (mcg/mL) | 10.0 | 7.1, 19.1 | 7 | 8.7 | 0.3, 12.0 | 5 | NS |
| sICAM-1 (ng/mL) | 416.1 | 403.5, 491.6 | 7 | 385.1 | 316.7, 506.6 | 5 | NS |
| | Endoscopic Remission | | | No Endoscopic Remission | | | |
| TNF-α (pg/mL) | 3.9 | 2.3, 6.4 | 9 | 15.6 | 11.5, 22.3 | 4 | 0.005 |
| α4β7 (ng/mL) | 12.1 | 5.4, 16.0 | 9 | 8.2 | 5.2, 12.4 | 4 | NS |
| α4β7 (change¹, ng/mL) | 11.9 | 4.8, 14.1 | 9 | 7.9 | 5.5, 12.7 | 3 | NS |
| MAdCAM1 (ng/mL) | 2.0 | 2.0, 3.1 | 9 | 2.4 | 2.0, 2.8 | 4 | NS |
| SAA (mcg/mL) | 27.5 | 6.1, 42.0 | 9 | 16.8 | 3.2, 32.7 | 4 | NS |
| sVCAM-1 (ng/mL) | 611.3 | 524.0, 879.3 | 9 | 728.4 | 660.0, 916.1 | 4 | NS |
| s-VCAM (change¹, ng/mL) | −70.7 | −109.7, −11.7 | 9 | −49.5 | −107.6, −42.4 | 3 | NS |
| CRP (mcg/mL) | 10.0 | 5.9, 13.5 | 9 | 5.2 | 1.3, 10.8 | 4 | NS |
| sICAM-1 (ng/mL) | 406.6 | 354.3, 506.6 | 9 | 418.6 | 338.2, 585.5 | 4 | NS |

¹change from baseline

TABLE 20
Associat4ions of week 14 biomarkers with maintenance outcomes.

| Biomarker | Median | IQR 25-75 | n | Median | IQR 25-75 | n | p value |
|---|---|---|---|---|---|---|---|
| | Clinical Remission | | | No Clinical Remission | | | |
| TNF-α (pg/mL) | 7.1 | 4.6, 7.5 | 7 | 8.0 | 6.7, 8.7 | 5 | NS |
| α4β7 (ng/mL) | 20.3 | 15.4, 23.5 | 7 | 6.0 | 4.7, 7.9 | 6 | 0.013 |
| α4β7 (change¹, ng/mL) | 17.0 | 13.9, 20.8 | 6 | 7.0 | 4.8, 8.8 | 4 | 0.045 |
| MAdCAM1 (ng/mL) | 3.3 | 2.1, 4.2 | 7 | 2.5 | 2.0, 3.5 | 6 | NS |
| SAA (mcg/mL) | 10.0 | 2.6, 15.1 | 7 | 25.7 | 13.9, 29.4 | 6 | NS |
| sVCAM-1 (ng/mL) | 598.0 | 424.0, 713.5 | 7 | 784.0 | 731.0, 887.3 | 6 | NS |
| s-VCAM (change¹, ng/mL) | −58.0 | −125.8, −6.8 | 6 | −127.5 | −168.3, −77.0 | 4 | NS |
| CRP (mcg/mL) | 4.9 | 2.8, 17.2 | 7 | 5.0 | 3.8, 10.4 | 6 | NS |
| sICAM-1 (ng/mL) | 330.0 | 301.5, 445.5 | 7 | 371.0 | 354.3, 506.3 | 6 | NS |
| | Endoscopic Remission | | | No Endoscopic Remission | | | |
| TNF-α (pg/mL) | 6.9 | 3.9, 7.5 | 10 | 5.3 | 2.5, 12.3 | 4 | NS |
| α4β7 (ng/mL) | 15.4 | 5.4, 21.8 | 10 | 6.1 | 5.9, 10.8 | 5 | NS |
| α4β7 (change¹, ng/mL) | 13.7 | 4.1, 19.4 | 9 | 8.3 | 7.0, 9.3 | 3 | NS |
| MAdCAM1 (ng/mL) | 2.7 | 2.0, 4.4 | 10 | 2.9 | 2.0, 3.7 | 5 | NS |
| SAA (mcg/mL) | 6.4 | 2.1, 17.1 | 10 | 23.9 | 23.1, 29.7 | 5 | 0.050 |
| sVCAM-1 (ng/mL) | 589.1 | 458.0, 760.3 | 10 | 746.0 | 726.0, 909.0 | 5 | 0.050 |
| s-VCAM (change¹, ng/mL) | −136.0 | −182.0, −21.0 | 9 | 1.0 | −51.0, 20.0 | 3 | NS |
| CRP (mcg/mL) | 4.5 | 1.1, 13.7 | 10 | 8.1 | 5.0, 12.2 | 5 | NS |
| sICAM-1 (ng/mL) | 316.5 | 299.3, 383.5 | 10 | 551.0 | 372.0, 566.0 | 5 | 0.020 |

¹change from baseline

TABLE 21
Associations of week 26 biomarkers with maintenance outcomes.

| Biomarker | Median | IQR 25-75 | n | Median | IQR 25-75 | n | p value |
|---|---|---|---|---|---|---|---|
| | Clinical Remission | | | No Clinical Remission | | | |
| TNF-α (pg/mL) | 3.8 | 2.3, 5.9 | 8 | 9.1 | 4.6, 18.6 | 8 | NS |
| α4β7 (ng/mL) | 14.1 | 10.9, 14.9 | 9 | 8.6 | 5.2, 10.3 | 10 | 0.050 |
| α4β7 (change¹, ng/mL) | 13.2 | 10.4, 13.9 | 5 | 4.4 | 2.2, 5.5 | 3 | 0.053 |
| MAdCAM1 (ng/mL) | 2.0 | 2.0, 2.1 | 8 | 2.5 | 2.0, 4.4 | 10 | NS |
| SAA (mcg/mL) | 2.6 | 1.1, 7.2 | 8 | 6.8 | 1.6, 26.2 | 10 | NS |
| sVCAM-1 (ng/mL) | 471.8 | 457.3, 666.3 | 8 | 633.7 | 513.1, 990.6 | 10 | NS |
| s-VCAM (change¹, ng/mL) | −84.2 | −118.8, 38.4 | 5 | 182.7 | 170.6, 213.4 | 3 | 0.025 |
| CRP (mcg/mL) | 1.1 | 0.6, 6.5 | 9 | 3.6 | 2.1, 10.5 | 10 | NS |
| sICAM-1 (ng/mL) | 360.9 | 315.3, 373.3 | 8 | 363.5 | 299.3, 924.2 | 10 | NS |
| | Endoscopic Remission | | | No Endoscopic Remission | | | |
| TNF-α (pg/mL) | 3.8 | 2.5, 8.6 | 10 | 9.1 | 5.8, 17.3 | 6 | NS |
| α4β7 (ng/mL) | 11.1 | 8.6, 14.5 | 12 | 8.3 | 5.8, 10.0 | 7 | NS |
| α4β7 (change¹, ng/mL) | 13.2 | 10.4, 13.9 | 5 | 4.4 | 2.2, 5.5 | 3 | 0.053 |
| MAdCAM1 (ng/mL) | 2.0 | 2.0, 2.5 | 11 | 2.1 | 2.0, 5.8 | 7 | NS |
| SAA (mcg/mL) | 2.6 | 1.1, 5.4 | 11 | 25.4 | 5.1, 31.4 | 7 | NS |
| sVCAM-1 (ng/mL) | 478.1 | 457.2, 626.1 | 11 | 690.4 | 579.3, 1129.0 | 7 | NS |
| s-VCAM (change¹, ng/mL) | −84.2 | −118.8, 38.4 | 5 | 182.7 | 170.6, 213.4 | 3 | 0.025 |
| CRP (mcg/mL) | 2.2 | 0.8, 4.7 | 12 | 7.1 | 1.8, 13.0 | 7 | NS |
| sICAM-1 (ng/mL) | 359.4 | 304.0, 371.6 | 11 | 412.8 | 311.5, 1095.9 | 7 | NS |

¹change from baseline

### TNF Concentrations

In all treated patients with baseline biomarkers measured, s-TNF concentrations significantly decreased from 9.1 pg/mL (n=17) at baseline to 3.8 pg/mL at week 26 (n=8, p=0.04). Compared with baseline values, s-TNF concentrations were numerically lower at other time points (p=NS). Using a linear mixed-effect model, s-TNF concentrations were found to decline more rapidly in endoscopic remitters (p=0.052). However, this did not achieve significance. Analysis at individual time points demonstrated significantly lower s-TNF concentrations at week 2 (6.7 pg/mL, IQR: 2.5-8.8 vs. 17.8 pg/mL, IQR: 12.1-24.5, p=0.038) and week 6 (3.9 pg/mL, IQR: 2.3-6.4 vs. 15.6 pg/mL, IQR: 11.5-22.3, p=0.005) in endoscopic remitters.

s-α4β7 Concentrations

In all treated-patients with baseline s-α4β7 measured (1.3 ng/mL, n=18), concentrations significantly increased at week 2 (14.1 ng/mL, n=11, p<0.001), week 6 (11.4 ng/mL, n=13, p<0.001), week 14 (12.7 ng/mL, n=13, p<0.001) and week 26 (9.9 ng/mL, n=8, p=0.002) compared with baseline. When analyzing (s)-α4β7 concentrations, a significant interaction between time and clinical remission (p=0.044) was found. Concentrations of s-α4β7 increased more rapidly in clinical remitters. A similar trend was found for endoscopic remitters (p=0.07).

Analysis at individual time points demonstrated week 14 s-α4β7 concentrations were significantly higher in clinical remitters (20.3 ng/mL, IQR: 15.4-23.5 vs. 6.0 ng/mL, IQR: 4.7-7.9, p=0.013). Furthermore, greater median increases in s-α4β7 concentrations from baseline to week 14 were associated with clinical remission (17.0 ng/mL, IQR: 13.9-20.8 vs. 7.0 ng/mL, IQR: 4.8-8.8, p=0.045). At ≥week 26, significantly higher s-α4β7 concentrations were observed in clinical remitters (14.1 ng/mL, IQR: 10.9-14.9 vs. 8.6 ng/mL, IQR: 5.2-10.3, p=0.050). Furthermore, a trend towards greater median increases in s-α4β7 concentrations from baseline to week 26 were observed for clinical remitters as well as for endoscopic remitters (13.2 ng/mL, IQR: 10.4-13.9 vs. 4.4 ng/mL, IQR: 2.2-5.5, p=0.053).

s-MAdCAM-1 Concentrations

In all treated patients with baseline s-MAdCAM-1 measured, compared with baseline (22.3 ng/mL, n=18), concentrations significantly decreased at week 2 (6.7 ng/mL, n=11, p<0.001), week 6 (3.4 ng/mL, n=13, p<0.001), week 14 (3.6 ng/mL, n=13, p<0.001) and week 26 (7.3 ng/mL, n=7, p=0.004). When analyzing s-MAdCAM-1 concentrations, significant interactions between time and clinical remission (p=0.006) and between time and endoscopic remission (p=0.005) were identified. s-MAdCAM-1 declined more rapidly in both clinical and endoscopic remitters compared with non-remitters. However, at each time point, s-MAdCAM-1 concentrations in remitters versus non-remitters were not associated with outcomes.

s-AA Concentrations

Concentrations of s-AA significantly decreased from baseline (45.5 mcg/mL, n=18) to week 14 (3.6 mcg/mL, p=0.025, n=13), and a downward trend was observed through week 26 (2.3 mcg/mL, p=0.1, n=8). Significant interactions between time and clinical remission or endoscopic remission with s-AA were not found. Comparison between endoscopic remitters and non-remitters at individual time points demonstrated that week 14 s-AA concentrations were significantly lower in endoscopic remitters (6.4 mcg/mL, IQR: 2.1-17.1 vs. 23.9 mcg/mL, IQR: 23.1-29.7, p=0.05).

s-VCAM-1 Concentrations

In all treated patients, s-VCAM-1 significantly decreased from baseline (719.3 ng/mL, n=18) to week 6 (637.8 ng/mL, n=13, p=0.002) and week 14 (622.5 ng/mL, n=13, p=0.003), but these changes did not persist at week 26 (695.0 ng/mL, n=8, p=NS). When analyzing s-VCAM-1 concentrations, there were significant interactions between time and clinical remission (p=0.001) as well as endoscopic remission (p<0.001). s-VCAM-1 declined more rapidly in clinical and endoscopic remitters. Week 14 s-VCAM-1 concentrations were significantly lower in endoscopic remitters (589.1 ng/mL, IQR:458.0-760.3 vs. 746.0 ng/mL, IQR: 726.0-909.0, p=0.05). Regarding changes at week 26 as compared with baseline, median s-VCAM-1 concentrations decreased in clinical and endoscopic remitters (−84.2 ng/mL, IQR: −118.8 to 38.4 vs. 182.7 ng/mL, IQR: 170.6 to 213.4 p=0.025), whereas s-VCAM-1 increased in non-remitters.

s-ICAM-1 Concentrations

In all treated patients with baseline biomarkers measured, s-ICAM-1 did not significantly change as compared with baseline. However, a significant interaction between time and endoscopic remission (p=0.014) with s-ICAM-1 was found. At individual time points, lower week 2 (385.9 ng/mL, IQR: 347.8-434.4 vs 508.6 ng/mL IQR: 443-693.9, p=0.042) and week 14 s-ICAM-1 concentrations were associated with endoscopic remission (316.5 ng/mL, IQR: 299.3-383.5 vs. 551.0 ng/mL IQR: 372.0-566.0, p=0.020).

### CRP Concentrations

In all treated patients with baseline biomarkers measured, CRP did not significantly change from baseline at any time point. There were no significant interactions between time and clinical remission (p=0.44) or endoscopic remission (p=0.75) with CRP. At individual time points, lower median CRP at week 2 was associated with endoscopic remission (p=0.017), but not at other time points.

### Correlations Between Biomarkers and Vedolizumab Concentration

Significant correlations between individual biomarker and VDZ concentrations were not consistently observed (Table 22).

TABLE 22
Correlations between individual biomarkers
and VDZ concentrations¹

| | |
|---|---|
| r value | p value |

Induction

| | | |
|---|---|---|
| s-TNF-α | −0.27 | 0.31 |
| s-α4β7 | 0.30 | 0.26 |
| s-MAdCAM-1 | −0.10 | 0.72 |
| CRP | −0.44 | 0.09 |
| s-AA | −0.45 | 0.08 |
| s-ICAM-1 | −0.28 | 0.29 |
| s-VCAM-1 | −0.45 | 0.08 |

Maintenance

| | | |
|---|---|---|
| s-TNF-α | −0.01 | 0.96 |
| s-α4β7 | −0.02 | 0.92 |
| s-MAdCAM-1 | −0.17 | 0.41 |
| CRP | −0.42 | 0.03 |
| s-AA | −0.35 | 0.08 |
| s-ICAM-1 | −0.33 | 0.10 |
| s-VCAM-1 | −0.30 | 0.14 |

¹Spearman correlation coefficients were calculated between VDZ and individual biomarker concentrations. This was performed separately during induction and maintenance using the latest available sample collection in each phase.

TNF concentrations did not correlate with s-α4β7, s-VCAM-1 or s-ICAM-1 (Table 23).

TABLE 23
Biomarker concentrations and systemic steroid use.¹

| | |
|---|---|
| r value | p value |

Baseline

| | | |
|---|---|---|
| s-α4β7 | 0.08 | 0.75 |
| s-ICAM-1 | 0.22 | 0.38 |
| s-VCAM-1 | 0.09 | 0.74 |

Induction

| | | |
|---|---|---|
| s-α4β7 | 0.18 | 0.49 |
| s-ICAM-1 | 0.17 | 0.51 |
| s-VCAM-1 | 0.13 | 0.63 |

Maintenance

| | | |
|---|---|---|
| s-α4β7 | −0.27 | 0.20 |
| s-ICAM-1 | 0.16 | 0.44 |
| s-VCAM-1 | 0.20 | 0.35 |

¹Spearman correlation coefficients were calculated between s-TNF and s-ICAM-1, s-VCAM-1 and s-α4β7. This was performed separately at baseline, induction, and maintenance using the latest available sample collection in each phase.

### Relationship Between Corticosteroid Use and Biomarkers Concentrations

Median baseline s-MAdCAM-1 concentrations were higher in patients with a corticosteroid requirement at baseline. Trends towards lower baseline s-TNF and s-α4β7 concentrations were seen in patients requiring corticosteroids at baseline. However, biomarker concentrations were not different between those requiring corticosteroid at week 26 and those not requiring corticosteroids at that time (Table 24).

TABLE 24
Biomarker concentrations and systemic corticosteroid use.¹

| | | |
|---|---|---|
| Systematic | No systematic | |
| steroid use | steroid use | p value |

Baseline biomarkers

| | | | | | |
|---|---|---|---|---|---|
| TNF-α (pg/mL) | 7.1 | (n = 12) | 13.3 | (n = 4) | 0.08 |
| α4β7 (ng/mL) | 0.2 | (n = 13) | 1.2 | (n = 4) | 0.09 |
| Madcam (ng/mL) | 24.1 | (n = 13) | 16.4 | (n = 4) | 0.04 |
| CRP (mcg/mL) | 11.3 | (n = 13) | 7.2 | (n = 4) | 0.78 |
| SAA (mcg/mL) | 31.8 | (n = 13) | 11.7 | (n = 4) | 0.55 |
| sICAM-1 ng/mL | 371.0 | (n = 13) | 408.5 | (n = 4) | 0.77 |
| sVCAM-1 (ng/mL) | 599.0 | (n = 13) | 941.0 | (n = 4) | 0.13 |

Week 26 biomarkers

| | | | | | |
|---|---|---|---|---|---|
| TNF-α (pg/mL) | 11.6 | (n = 6) | 8.5 | (n = 17) | 0.76 |
| α4β7 (ng/mL) | 5.6 | (n = 7) | 1.3 | (n = 19) | 0.25 |
| Madcam (ng/mL) | 2.1 | (n = 7) | 12.2 | (n = 19) | 0.31 |
| CRP (mcg/mL) | 6.5 | (n = 7) | 7.9 | (n = 19) | 1.00 |
| SAA (mcg/mL) | 26.5 | (n = 7) | 9.2 | (n = 18) | 0.20 |
| sICAM-1 ng/mL | 372 | (n = 7) | 384.5 | (n = 18) | 0.69 |
| sVCAM-1 (ng/mL) | 726 | (n = 7) | 620.5 | (n = 18) | 0.50 |

¹Median baseline and week 26 biomarker concentrations are compared with baseline and week 26 requirements for systemic steroids.

### Analysis of Results

This example demonstrated the association between serum biomarkers with prospectively scored outcomes during maintenance VDZ therapy in UC patients. Profile changes in s-α4β7 concentrations were uniquely analyzed during VDZ therapy in all treated patients and differences between remitters and non-remitters were described. s-α4β7 concentrations increased by week 2 and remained elevated, plateauing at weeks 6, 14 and 26. s-α4β7 increased more rapidly and was preferentially higher at individual time points in remitters. Notably, corticosteroid use and s-TNF concentrations did not affect s-α4β7 concentrations. Total s-α4β7 measurements used a sandwich ELISA that utilizes a distinct binding site from VDZ. This allowed for detection of a4β7 that is both bound and not bound to VDZ. Thus, this may represent a convenient surrogate for a4β7 expression on lymphocytes in patients receiving vedolizumab therapy.

s-VCAM-1 decreased consistently at each individual time point after week 6, and although a significant difference was not observed at week 26, there was a smaller sample size at this time point. Consistent with these findings, s-VCAM-1 concentrations of declined more rapidly in remitters compared to non-remitters. Furthermore, remitters had consistently greater reductions in s-VCAM-1 maintenance concentrations compared to baseline. Although s-ICAM-1 was inconsistently lower at certain individual time points in remitters, concentrations of declined more rapidly in remitters. Inconsistencies of data at individual time points may be accounted by sample size considerations, thus the linear mixed-effect model better accounted for rates of change in biomarker concentrations over time in each group. Furthermore, later changes in s-VCAM-1 concentrations may be related to the time required for adaptive changes to occur.

Serum s-VCAM-1 and s-ICAM-1 are both induced by TNF, and these relationships are independent of transmembrane intestinal CAMs. See, Podolsky, D. K., et al. J Clin Invest. 1993, 92(1), 372-380; Jones, S. C., et al. Gut. 1995, 36(5), 724-730; Goke, M., et al. J Gastroenterol. 1997, 32(4), 480-486; Giorelli, M., et al. Cell Commun Adhes. 2002, 9(5-6), 259-272; Iwao, M., et al. Biochem Biophys Res Commun. 2004, 317(3), 729-735. Thus, relationships between CAM with s-TNF and corticosteroids were explored. Lower s-VCAM-1 and s-ICAM-1 concentrations in remitters were independent of s-TNF concentrations or corticosteroid use.

Both s-TNF and s-AA decreased in the entire VDZ-treated cohort, but only consistently trended downward after week 6 and achieved significance during maintenance. Since TNF is an inflammatory mediator and s-AA is an acute phase reactant, these markers may represent a patient's inflammatory burden. Interestingly, while a more rapid decline in s-TNF was observed in endoscopic remitters, this was not seen with s-AA. This is consistent with the notion that VDZ does not alter acute phase reactants differentially based on outcome, such as CRP, and implies that alternative inflammatory mediators such as s-TNF may be a useful surrogate biomarker. See, Sandborn W. J., et al. N. Engl J Med 2013, 369, 711-721; Sands B. E., et al. Gastroenterology. 2014, 147(3), 618-627.e3; Amiot, A., et al. Clin Gastroenterol Hepatol. 2016, 14(11), 1593-1601.e2.

Concentrations of s-MAdCAM-1 decreased during the course of VDZ therapy in all patients and declined more rapidly in remitters. Notably, s-MAdCAM-1 concentrations were not consistently associated with corticosteroid-use. VDZ concentrations were not associated with outcomes. Furthermore, the lack of correlation between biomarkers and VDZ concentrations suggests that the associations of biomarkers with outcomes are independent of drug concentrations for the studied doses of VDZ.

In conclusion, this study reports the association of serum biomarkers with outcomes in VDZ-treated UC patients. s-α4β7, s-TNF, s-MAdCAM-1 and s-AA significantly changed with VDZ therapy by week 26 compared to baseline. s-α4β7 increased, while s-TNF, s-MAdCAM-1, s-ICAM-1, and s-VCAM-1 declined, more rapidly in remitters. During induction time points, s-TNF concentrations were lower in remitters. During maintenance time points, s-α4β7 was consistently higher and s-VCAM-1 was consistently lower in remitters.

All publications and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A method for predicting that a subject having inflammatory bowel disease (IBD) will have a clinical response to an anti-α4β7 integrin drug or reach remission during the course of therapy, the method comprising:
(a) administering to a subject having IBD an anti-α4β7 integrin drug during an induction phase;
(b) assessing the concentration of the anti-α4β7 integrin drug at the induction phase in a sample from the subject; and
(c) determining whether the subject will have a clinical response or reach remission at a later time point based upon the concentration of the anti-α4β7 integrin drug at the induction phase.

2. The method of claim 1, wherein the inflammatory bowel disease is ulcerative colitis (UC) or Crohn's Disease (CD).

3. The method of claim 1, wherein the anti-α4β7 integrin drug is ENTYVIO® (vedolizumab, VDZ).

4. The method of claim 1, wherein the induction phase is between week 0 and week 6 of the course of therapy.

5. The method of claim 1, wherein the later time point is at weeks 8, 10, 12, 14, 16, 20. 22, 24, 30, 32, 40, 48, or 52 during the course of therapy.

6. The method of claim 1, wherein a concentration of VDZ of ≥23.2 μg/ml at week 2 is associated with a clinical response or remission at week 14, 22, 30 and 52.

7. The method of claim 1, wherein a concentration of VDZ of ≥19.8 μg/ml at week 6 is associated with a clinical response or remission at week 14, 22, 30 and 52.

8. The method of claim 1, wherein the clinical response or remission is a member selected from the group consisting of steroid free remission, clinical remission, normalized C-reactive protein (CRP), no steroid use in 4 weeks, and endoscopic remission.

9. The method of claim 1, wherein a concentration of VDZ is negatively correlated to concentration of CRP at week 14 and 22.

10. The method of claim 1, wherein the concentration of VDZ at induction is used to identify a subject that will have a clinical response or reach remission.

11. A method for predicting that a subject having inflammatory bowel disease (IBD) being administered an anti-α4β7 integrin drug therapy will reach remission, the method comprising:
(a) assessing the concentration of the anti-α4β7 integrin drug in a sample from the subject during a maintenance phase; and
(b) determining whether the subject will reach remission at a later time point based upon the concentration of the anti-α4β7 integrin drug during the maintenance phase.

12. The method of claim 11, wherein the inflammatory bowel disease is ulcerative colitis (UC) or Crohn's Disease (CD).

13. The method of claim 11, wherein the anti-α4β7 integrin drug is ENTYVIO® (vedolizumab, VDZ).

14. The method of claim 11, wherein an induction phase is between week 0 and week 6 of the course of therapy, and the maintenance phase is after 6 weeks.

15. The method of claim 11, wherein the remission is a member selected from the group consisting of steroid free remission, clinical remission, normalized C-reactive protein (CRP), no steroid use in 4 weeks, and endoscopic remission.

16. The method of claim 11, wherein a concentration of VDZ of ≥12 μg/ml after week 6 is associated with remission at week 14, 22, 30, and 52, or later.

17. The method of claim 11, wherein a concentration of VDZ of ≥13 μg/ml after week 6 is associated with remission at week 14, 22, 30, and 52, or later.

18. The method of claim 11, wherein a concentration of VDZ of ≥14 μg/ml after week 6 is associated with remission at week 14, 22, 30, and 52, or later.

19. The method of claim 11, wherein a concentration of VDZ of ≥15 μg/ml after week 6 is associated with remission at week 14, 22, 30, and 52, or later.

20. A method for predicting whether a subject having inflammatory bowel disease (IBD) will be a remitter to an anti-α4β7 integrin drug treatment regimen, the method comprising:
(a) detecting the presence or level of at least one predictive marker selected from the group consisting of s-TNFα, s-α4β7, s-MAdCAM-1, s-CRP, s-AA, s-VCAM-1, s-ICAM-1, and a combination thereof, in a sample from the subject; and
(b) classifying the subject as a remitter or a non-remitter to the anti-α4β7 integrin drug treatment according to a predictive marker profile based on a higher or lower level of the at least one predictive marker compared to a corresponding reference value.

21. The method of claim 20, wherein the inflammatory bowel disease is ulcerative colitis (UC) or Crohn's Disease (CD).

22. The method of claim 20, wherein the anti-α4β7 integrin drug is ENTYVIO® (vedolizumab, VDZ).

23. The method of claim 20, wherein s-α4β7 is increased in remitters.

24. The method of claim 20, wherein one or more members selected from the group consisting of s-TNFα, s-MAdCAM-1, s-ICAM-1, and s-VCAM-1 is lower in remitters.

25. The method of claim 20, wherein during induction time points, s-TNFα concentrations are lower in remitters.

26. The method of claim 20, wherein during maintenance time points, s-α4β7 is higher in remitters and s-VCAM-1 is lower in remitters.

27. The method of claim 20, wherein the method includes least two predictive markers.

28. The method of claim 20, wherein the method includes least three predictive markers.

29. The method of claim 20, wherein the method includes least four predictive markers.

30. The method of claim 20, wherein the method includes least five predictive markers.

31. The method of claim 20, wherein the method includes least six predictive markers.

32. The method of claim 20, wherein the method includes seven predictive markers.
